(19) United States
(12) Patent Application Publication
(10) Pub. No.: US 2008/0038723 A1
(43) Pub. Date: Feb. 14, 2008

(54) Functional assessment of DNA mismatch repair gene variants

(76) Inventors: Grant A. Bitter, Agoura, CA (US); Aaron R. Ellison, Thousand Oaks, CA (US)

Correspondence Address: Richard J. Mazza;c/o BitTech, Inc., P.O. Box 1499, Agoura Hills, CA, 91376-1499, US

(21) Appl. No.: 10/863,872

(22) Filed: Jun. 7, 2004

(51) Int. Cl. C12Q 1/68; C07K 14/395; C12N 1/19; C12N 15/31

(52) U.S. Cl. 435/6; 435/254.21; 436/94; 530/358; 536/23.74

(57) ABSTRACT

Methods and materials are described for determining the susceptibility of an individual to diseases associated with defects in DNA mismatch repair function, principally human colorectal and other cancers, by the use of activity assays to assess the functional significance of mutations in genes encoding DNA mismatch repair proteins. These methods allow the prospective identification of amino acid substitutions, corresponding to naturally occurring genetic mutations, which impair human DNA mismatch repair function and may lead to oncogenic consequences. Certain irregular sequences encoding protein sequences that differ from native DNA mismatch repair proteins, and which may foretell a higher probability for developing cancer and other genetically based diseases, have been now been newly identified by these methods.

### ACKNOWLEDGEMENT

Work taking place in the laboratory when this invention occurred was supported in part by a research grant from the National Institutes of Health (R44CA81965). The U.S. Government may have rights in this invention as a result of this support.

### BACKGROUND OF THE INVENTION

1. Technical Field

This invention relates to the diagnosis in humans of susceptibility to the development of colorectal cancer and other diseases associated with the loss of function in DNA mismatch repair in vivo.

2. Background

Hereditary nonpolyposis colorectal cancer (HNPCC) is an autosomal dominant inherited disease caused by defects in the process of DNA mismatch repair, and mutations in the hMLH1 or hMSH2 genes are responsible for the majority of HNPCC. In addition to clear loss-of-function mutations conferred by nonsense or frameshift alterations in the coding sequence or by splice variants, genetic screening has revealed a large number of missense codons with less obvious functional consequences. The ability to discriminate between a loss-of-function mutation and a silent polymorphism (i.e. no apparent loss of function) is important for genetic testing for inherited diseases like HNPCC where there exists opportunity for early diagnosis and preventive intervention.

Colorectal cancer (CRC) is one of the most common cancers, by some estimates affecting 3-5% of the population in developed countries by age 70. Hereditary nonpolyposis colorectal cancer (HNPCC) accounts for 2-8% of all CRC, depending on the population and clinical criteria used, and is manifested by a high rate of mortality in the absence of early detection and treatment (reviewed in: (1-6). Diagnosis of HNPCC in a family is based on kindred analysis using the Amsterdam Criteria (7), which require: i) three or more family members to have had histologically verified CRC, with one being a first-degree relative of the other two, ii) CRC in at least two generations, and iii) at least one individual diagnosed with CRC before age 50. At the molecular level, HNPCC is associated with defects in the cellular process of DNA mismatch repair.

The process of DNA mismatch repair (corrects non-native (i.e., irregular or mutant) DNA structures that form primarily during DNA replication. These aberrant structures include incorrectly paired bases resulting from misincorporation by DNA polymerases, as well as insertion/deletion loops in DNA which form, for example, as a result of microsatellite instability. Microsatellite sequences comprise a tract of repetitive nucleotides within a DNA sequence, for example, -GGGGGGGGGGGG- or -ACACACACAC-. In cells with dysfunctional MMR, microsatellite sequences are highly unstable and thus are prone to mutate during DNA replication. The amino acid sequences of MMR protein functional domains are conserved from E. coli to humans, and the eukaryotic MMR proteins are named based on their homology to E. coli MutS and MutL. Mechanistic studies of MMR in yeast and human cells have elucidated similar processes (reviewed in (8-10)). MutSα is a heterodimer of MSH2 and MSH6, while MutSβ is a heterodimer of MSH2 and MSH3. MutSα recognizes base:base mismatches, as well as single base insertion/deletion mispairs. MutSβ also recognizes single base insertion/deletion mispairs but is primarily responsible for recognition of larger insertion/deletion mispairs. Heterodimers of the MutL homologues bind to the MutSα or MutSβ DNA mismatch complex to effect repair. The yeast MLH1-PMS1 heterodimer (MLH1-PMS2 in humans) binds both MutSα and MutSβ, while the yeast MLH1-MLH3 complex (MLH1-PMS1 in humans) binds MutS, (reviewed in (10)).

HNPCC has been shown to be caused by mutations in the hMLH1, hMSH2, hPMS1, hPMS2, hMLH3 and hMSH6 genes (5). Hundreds of mutations of all types have been described with approximately 90% occurring in either hMLH1 or hMSH2. It is probable that the majority of HNPCC is associated with mutations in hMLH1 and hMSH2 since inactivation of either of these genes results in impaired replication of a broad spectrum of mismatches (single base:base mismatches and both small and large insertion/deletion loops). The most comprehensive public database of sequence alterations observed in genes encoding human MMR proteins and implicated in HNPCC is the International Collaborative Group (ICG) on HNPCC (http://www.nfdht.nl). Additional sequence variants which have been observed also appear in the Human Gene Mutation Database (http://www.hgmd.org) and the Swiss Protein Database (http://us.expasy.org) as well as several single nucleotide polymorphism (SNP) databases (http://dir-apps.niehs.nih.gov/egsnp/, http://www.genome.utah.edu/genesnps/, http://www.ncbi.nlm.nkh.gov/SNP). In addition to mutations in hMLH1 and hMSH2, it has been reported that defects in MMR can be caused by gene silencing due to hypermethylation (11). Genetic testing of individuals in HNPCC kindreds should decrease cancer-associated morbidity and mortality in this group. Removal of pre-cancer polyps observed during colonoscopy is highly effective in preventing the progression of nonpolyposis colorectal cancer. By identification of those individuals with MMR defects in HNPCC kindreds, routine colonoscopies can be performed with, and restricted to, those individuals who will derive benefits from the procedure.

In the genetic analyses of HNPCC kindreds, more than 25% of the gene alterations observed are minor variants such as amino acid substitutions or small in-frame deletions. These sequence variants, furthermore, are scattered throughout the gene coding region. If an observed amino acid replacement can be shown to segregate with disease in the affected family, it suggests that the amino acid substitution is an inactivating mutation. Frequently, however, small family size or unavailability of clinical samples has precluded attempts to correlate the amino acid replacement with pathogenic effect. As genetic analyses of HNPCC kindreds has continued, an increasing number of minor variants have been documented. To date, missense codons resulting in 164 different amino acid substitutions have been described in hMLH1 while 150 have been reported in hMSH2. It is now generally acknowledged (3, 6, 9, 12) that accurate and effective genetic testing for HNPCC will require methods to determine the functional significance of these minor variants, since the utility of genetic tests is severely compromised if there is any ambiguity in the results.

It is now clear that cancer is an acquired disease in which cells evolve in a stepwise manner from a normal state to premalignancy to invasiveness (13, 14). This progression (tumorigenesis) is likely to occur over long periods of time (typically, 15-30 years), and it results in age-dependent increases in cancer incidence. As cancer cells divide they acquire the necessary capabilities for self-sufficiency in growth signals, insensitivity to anti-growth signals, protection from apoptosis, limitless replicative potential (immortality), sustained angiogenesis, and tissue invasiveness (14). While the order and biological mechanisms for the acquired capabilities may vary, one universal characteristic of cancer cells is that their genomes contain a large number of mutations (15-18). These mutations appear to lay the genetic foundation for the acquisition of capabilities that permit tumorigenesis.

Although it has been debated whether elevated mutation rates are essential for tumorigenesis, it is generally agreed that events which increase the number of mutations in a cell will lead to an increased risk of developing cancer. This correlation between the acquisition of mutations, either at the nucleotide sequence or chromosomal level, and tumorigenesis is well-established and the basis for currently-recommended practices in cancer avoidance and prevention. For example, there is a causal link between exposure to physical and chemical mutagens (such as tobacco products, ultraviolet light, and radioactivity) and tumorigenesis (19-21). At the biochemical level these mutagens are known to cause DNA damage and to alter a cell's genetic information in ways that appear to facilitate tumorigenesis. Also, an accumulation of mutations may occur via malfunctioning DNA repair pathways. Normally, cells have several mechanisms for preserving their genetic integrity, including MMR, nucleotide excision repair, base excision repair, double-strand break repair, and photoreactivation. These mechanisms are necessary for dealing with the errors that occur at a low frequency through normal cellular metabolism, DNA replication and the environment. However, if cells are not able to repair damaged DNA, the altered DNA sequence, i.e., a mutation, becomes an enduring feature in cells of that lineage. Therefore, conditions which decrease the efficiency of DNA repair (i.e., increase the frequency at which mutations accumulate) will undoubtedly increase the likelihood that cells will more rapidly acquire the capabilities necessary for tumorigenesis. Proof of these principles has been borne out most convincingly in humans by the discovery of certain inherited cancer-susceptibility syndromes, in which individuals that carry germline mutations in the genes for DNA repair have a much greater susceptibility to develop cancer than individuals in the general population. For example, patients with xeroderma pigmentosum (XP) have been shown to carry defects in the genes encoding factors for nucleotide excision repair (22). These patients have an increased risk [to develop] of developing skin cancer as a result of being unable to repair the DNA lesions caused by exposure to UV light. As described in detail previously, patients with HNPCC carry mutations in the genes encoding proteins that carry out M (including MSH2, MLH1, and MSH6). These patients have an increased risk of developing colorectal, endometrial and other types of cancers as a result of being unable to carry out MMR. Taken together, these fundamental concepts establish a causative link between events that increase the number of mutations in a cell and the potential for those cells to acquire the essential capabilities for tumorigenesis and thus greaten an individual's susceptibility to cancer development.

### SUMMARY OF THE INVENTION

The present invention provides for the identification of certain partial or complete inactivations of human genes encoding proteins involved in DNA mismatch repair. This identification is carried out by use of quantitative in vivo DNA mismatch repair assays (utilizing the yeast Saccharomyces cerevisiae, for example) which determine the functional significance of amino acid substitutions observed in humans.

This invention features a diagnostic method for determining whether an individual, i.e., a human subject, carries a mutation in a gene which encodes a protein involved in DNA mismatch repair. In general, the approach is based on an in vivo functional analysis of variant DNA mismatch repair genes which have been introduced into cells of the yeast Saccharomyces cerevisiae that lack a functional copy of the corresponding native DNA mismatch repair gene. This method differs from work described earlier (WO 02/081624 A3, published Oct. 17, 2002) by featuring a new approach for the prospective identification of MMR gene variants having inactivating missense mutations (described in greater detail further below in this text), and new hybrid human-yeast DNA molecules for the analysis of human sequence alterations in yeast. Cumulatively, 180 mismatch repair protein variants, each having one amino acid substitution compared to the wild-type sequence, have been developed and assayed for function in DNA mismatch repair. The present method is useful for the diagnosis of presusceptibility to diseases that are associated with defects in MMR function, a notable example of which is cancer.

In general, in one of its primary aspects the present invention provides a diagnostic method for determining whether a human subject has an increased rate of accumulating genetic mutations due to the loss of DNA mismatch repair function associated with any of the following amino acid sequences:

sequences corresponding to human MLH1: 23D (SEQ ID NO: 262), 29I (SEQ ID NO: 263), 38T (SEQ ID NO: 264), 40F (SEQ ID NO: 265), 40N (SEQ ID NO: 266), 40T (SEQ ID NO: 267), 41E (SEQ ID NO: 268), 41G (SEQ ID NO: 269), 41N (SEQ ID NO: 270), 42E (SEQ ID NO: 271), 42T (SEQ ID NO: 272), 42V (SEQ ID NO: 273), 43A (SEQ ID NO: 274), 43D (SEQ ID NO: 275), 43E (SEQ ID NO: 276), 43F (SEQ ID NO: 277), 43H (SEQ ID NO: 278), 43I (SEQ ID NO: 279), 43L (SEQ ID NO: 280), 43M (SEQ ID NO: 281), 43P (SEQ ID NO: 282), 43S (SEQ ID NO: 283), 43T (SEQ ID NO: 284), 43V (SEQ ID NO: 285), 43W (SEQ ID NO: 286), 43Y (SEQ ID NO: 287), 44D (SEQ ID NO: 288), 44G (SEQ ID NO: 289), 44K (SEQ ID NO: 290), 44M (SEQ ID NO: 291), 44N (SEQ ID NO: 292), 45I (SEQ ID NO: 293), 46T (SEQ ID NO: 294), 47S (SEQ ID NO: 295), 47T (SEQ ID NO: 296), 48G (SEQ ID NO: 297), 48Y (SEQ ID NO: 298), 49E (SEQ ID NO: 299), 49M (SEQ ID NO: 300), 49N (SEQ ID NO: 301), 51A (SEQ ID NO: 302), 51D (SEQ ID NO: 303), 55S (SEQ ID NO: 304), 56M (SEQ ID NO: 305), 56P (SEQ ID NO: 306), 57N (SEQ ID NO: 307), 59F (SEQ ID NO: 308), 59H (SEQ ID NO: 309), 59N (SEQ ID NO: 310), 59T (SEQ ID NO: 311), 61N (SEQ ID NO: 312), 63G (SEQ ID NO: 313), 63Y (SEQ ID NO: 314), 64I (SEQ ID NO: 315), 64S (SEQ ID NO: 316), 65A (SEQ ID NO: 317), 65D (SEQ ID NO: 318), 65E (SEQ ID NO: 319), 65S (SEQ ID NO: 320), 65V (SEQ ID NO: 321), 67W (SEQ ID NO: 322), 68F (SEQ ID NO: 323), 68N (SEQ ID NO: 324), 68S (SEQ ID NO: 325), 70I (SEQ ID NO: 326), 70N (SEQ ID NO: 327), 72G (SEQ ID NO: 328), 73M (SEQ ID NO: 329), 73P (SEQ ID NO: 330), 74L (SEQ ID NO: 331), 76E (SEQ ID NO: 332), 77S (SEQ ID NO: 333), 77Y (SEQ ID NO: 334), 79W (SEQ ID NO: 335), 80I (SEQ ID NO: 336), 80S (SEQ ID NO: 337), 80V (SEQ ID NO: 338), 82K (SEQ ID NO: 339), 82M (SEQ ID NO: 340), 82S (SEQ ID NO: 341), 83F (SEQ ID NO: 342), 83P (SEQ ID NO: 343), 89G (SEQ ID NO: 344), 89V (SEQ ID NO: 345), 91V (SEQ ID NO: 346), 99I (SEQ ID NO: 347), 99L (SEQ ID NO: 348), 100P (SEQ ID NO: 349), 100Q (SEQ ID NO: 350), 101D (SEQ ID NO: 351), 102D (SEQ ID NO: 352), 102G (SEQ ID NO: 353), 103T (SEQ ID NO: 354), 103V (SEQ ID NO: 355), 111P (SEQ ID NO: 356), 111T (SEQ ID NO: 357), 113A (SEQ ID NO: 358), 114I (SEQ ID NO: 359), 115E (SEQ ID NO: 360), 115F (SEQ ID NO: 361), 115N (SEQ ID NO: 362), 115S (SEQ ID NO: 363), 116A (SEQ ID NO: 364), 118N (SEQ ID NO: 365), 128P (SEQ ID NO: 366), 182G (SEQ ID NO: 367), 193P (SEQ ID NO: 368), 304V (SEQ ID NO: 601), 542P (SEQ ID NO: 369), 549P (SEQ ID NO: 370), 640S (SEQ ID NO: 602), 663G (SEQ ID NO: 371), 755S (SEQ ID NO: 372), 22A (SEQ ID NO: 598), 29S (SEQ ID NO: 373), 32V (SEQ ID NO: 374), 36L (SEQ ID NO: 375), 43C (SEQ ID NO: 376), 43G (SEQ ID NO: 377), 43N (SEQ ID NO: 378), 43Q (SEQ ID NO: 379), 43R (SEQ ID NO: 380), 62R (SEQ ID NO: 381), 64D (SEQ ID NO: 382), 71D (SEQ ID NO: 383), 75T (SEQ ID NO: 384), 95T (SEQ ID NO: 385), 136S (SEQ ID NO: 386), 141R (SEQ ID NO: 599), 160V (SEQ ID NO: 387), 272V (SEQ ID NO: 388), 286Q (SEQ ID NO: 600), 441T (SEQ ID NO: 389), 648L (SEQ ID NO: 390), and 659Q (SEQ ID NO: 391).

sequences corresponding to human MSH2: 100/101-del (SEQ ID NO: 604), 198G (SEQ ID NO: 392), 199R (SEQ ID NO: 400), 272V (SEQ ID NO: 393), 333R (SEQ ID NO: 90), 338R (SEQ ID NO: 607), 439-del (SEQ ID NO: 609), 440P (SEQ ID NO: 610), 503P (SEQ ID NO: 394), 534C (SEQ ID NO: 611), 595R (SEQ ID NO: 614), 603N (SEQ ID NO: 615), 622T (SEQ ID NO: 616), 636P (SEQ ID NO: 99), 639R (SEQ ID NO: 93), 683R (SEQ ID NO: 395), 692R (SEQ ID NO: 95), 697R (SEQ ID NO: 96), 751R (SEQ ID NO: 97), 30L (SEQ ID NO: 603), 44M (SEQ ID NO: 396), 61P (SEQ ID NO: 397), 127S (SEQ ID NO: 398), 167H (SEQ ID NO: 399), 186S (SEQ ID NO: 89), 199W (SEQ ID NO: 605), 322V (SEQ ID NO: 606), 323C (SEQ ID NO: 401), 333Y (SEQ ID NO: 91), 349L (SEQ ID NO: 608), 390F (SEQ ID NO: 402), 390V (SEQ ID NO: 403), 562V (SEQ ID NO: 612), 583S (SEQ ID NO: 613), 609V (SEQ ID NO: 92), 647K (SEQ ID NO: 100), 656H (SEQ ID NO: 101), 683V (SEQ ID NO: 404), 688I (SEQ ID NO: 405), 691T (SEQ ID NO: 94), 722I (SEQ ID NO: 617), 729V (SEQ ID NO: 102), 735V (SEQ ID NO: 406), 770V (SEQ ID NO: 98), and 845E (SEQ ID NO: 407).

This diagnostic method is especially useful in practical applications for determining whether a human subject has an increased susceptibility to the development of cancer (e.g., colorectal, endometrial, ovarian) associated with loss of DNA mismatch repair function by determining whether that subject possesses a gene which encodes a DNA mismatch repair protein having any of the above mentioned amino acid sequences and detecting if that sequence is an inactivating mutation or an efficiency polymorphism, either of which carries a greater than normal risk of cancer development.

Other aspects of the invention include biological and biochemical materials which are useful in the practice of the methods of the invention. These materials and their application are described in detail further below.

### BRIEF DESCRIPTION OF THE FIGURES

FIG. 1 is a schematic representation of proteins encoded by the hybrid human-yeast MLH1 genes. Portions of the hybrid representing human sequences are represented with filled bars. Numbers above each bar indicate the corresponding amino acid residues at the junction between human and yeast sequences. The MMR defect (normalized to the strain expressing wild-type yeast MLH1) is listed to the right of each protein.

FIG. 2 is a schematic representation of proteins encoded by the hybrid human-yeast MSH2 genes. Portions of the hybrid representing human sequences are represented with filled bars. Numbers above each bar indicate the corresponding amino acid residues at the junction between human and yeast sequences. The MMR defect (normalized to the strain expressing wild-type yeast MSH2) is listed to the right of each protein. The indication “ins9” refers to an insertion of the yeast MSH2 coding sequence for amino acids 827-KNLKEQKHD-835 between human residues 807-808.

FIG. 3 is comprised of two parts. FIG. 3A shows the sequence of the 5′ end of the ADE2-MS3 reporter gene. The microsatellite (AC)₁₉A(G)₁₈ was introduced between the ATG initiator codon and the second codon (GAT) of the native ADE2 gene as described in Example 6. The fragment was transformed into strain into YBT24, replacing the native ADE2 locus to generate strain YBT41. FIG. 3B is schematic representation of the prospective screen (Methods “a” and “b”; Example 8) for inactivating mutations in MLH1. Fragments of the human-yeast hybrid genes pMLH1_h(41-86) or pMLH1_h(77-134) were generated by error-prone PCR, mixed with a ClaI-AatII-digested pMLH1 expression vector transformed into strain YBT41. Circular plasmids are formed in vivo by homologous recombination between the PCR product and gapped vector, and transformants are selected on plates lacking histidine and containing low concentrations of adenine. Clones with mutant mlh1 genes are identified by red sectoring and the plasmids are recovered to determine the DNA sequence of the mutagenized gene.

FIG. 4 comprises four photographs of yeast strain YBT41. Each photograph shows YBT41 following transformation with a different MLH1 expression vector and growth on plates containing low adenine (4 μg/ml). As, indicated above each panel, yeast YBT41 was transformed with the expression vector pMETc, containing no MLH1 gene; pMLH1, which contains the wild-type yeast MLH1 gene; and pMLH1_h(41-86) or pMLH1_h(77-134), which contain a hybrid human-yeast MLH1 gene. White colonies with red sectoring indicates a high level of microsatellite instability (mutation to ade2; mutant cells are red due to an accumulation of an intermediate in adenine biosynthesis).

FIG. 5. Yeast strain YBT24 containing pSH91 was transformed with pMLH1_h(41-86), pMLH1_h(77-134), variants of these plasmids isolated in the prospective screen and the expression vector pMETc lacking an MLH1 gene. Mutation frequencies were determined using the standardized quantitative MMR assay as described in Example 1. The mean mutation frequency ±standard deviation of two independent cultures is shown. The species (“human” or “yeast”, in parenthesis) indicates whether the missense mutation is in the human or yeast portion of the hybrid. FIG. 5A: Mutation frequencies of pMLH1_h(41-86) variants and controls: pMLH1_h(41-86) S44F (human), 2.7×10⁻³; I47S (human), 2.3×10⁻³; L56P (human), 2.7×10⁻³; I59T (human), 2.1×10⁻³; D63Y (human), 2.3×10⁻³; I68N (human), 2.5×10⁻³; V110A (yeast), 2.4×10⁻³; pMLH1_h(41-86), 2.9×10⁻⁴; pMETc, 2.3×10⁻³. FIG. 5B: Mutation frequencies of pMLH_h(77-134) variants and controls: pMLH1_h(77-134) L56H (yeast), 2.5×10⁻³; N61S (yeast), 3.2×10⁻³; G62E (yeast), 3.6×10⁻³; A103T (human), 2.0×10⁻³; T114I (human), 2.0×10⁻³; T115S (human), 3.4×10⁻³; K118N (human), 4.6×10⁻³; pMLH1_h(77-134), 1.4×10⁻⁴; pMETc, 2.0×10⁻³.

FIG. 6 shows an alignment of MLH1 orthologs and the position of all loss-of-MMR function missense mutations isolated in the prospective screen. The 117 unique substitutions isolated are represented above the appropriate residue in the human sequence. N-terminal MLH1p sequences from human (Hs, H. sapiens), mouse (Mm, M. musculus), rat (Rn, R. norvegicus), fruit fly (Dm, D. melanogaster), yeast (Sc, S. cerevisiae and Sp, S. pombe), plant (At, A. thaliana), flatworm (Ce, C. elegans), and bacteria (Sa, S. aureus and Ec, E. coli) were aligned using ClustalW (http://www.ebi.ac.uk/clustalw/). Conserved residues are highlighted. Structural features, including α-helices (stippled boxes) and β-strands (arrows), in the E. coli MutL polypeptide (23) are indicated below the alignment. Barbells represent the location of the ATP binding motifs (I-IV), which are conserved in GHL ATPases (23, 24). Underlined residues represent sites having nonsynonomous polymorphisms which may predispose individuals to develop HNPCC. Boxed residues (substitutions) were isolated in this study and are equivalent to substitutions found in the human population and associated with HNPCC (http://www.nfdht.nl).

FIG. 7 depicts mutation frequencies conferred by missense substitutions at human MLH1p residue 44 (S44). Yeast strain YBT24 containing pSH91 was transformed with pMLH1_h(41-86), variants of this plasmid containing the indicated mutation and the expression vector pMETc lacking an MLH1 gene. Mutation frequencies were determined using the standardized quantitative MMR assay as described in Example 1. The mean mutation frequency ±standard deviation of two to twelve independent cultures is shown. Cells containing the parental hybrid pMLH1_h(41-86) exhibited a mutation frequency of 2.7×10⁻⁴. The mutation defect (shown above each bar) for each variant and control was calculated by dividing the mutation frequency of cells expressing the variant by the mutation frequency of cells expressing parental hybrid pMLH1_h(41-86). A MLH1_h(41-86) gene containing a termination codon at position 44 and 45 is referred to as “S44-Term”.

FIG. 8 depicts mutation frequencies conferred by missense substitutions at human MLH1p residue 43 (K43). Yeast strain YBT24 containing pSH91 was transformed with pMLH1_h(41-86), variants of this plasmid containing the indicated mutation and the expression vector pMETc lacking an MLH1 gene. Mutation frequencies were determined using the standardized quantitative MMR assay as described in Example 1. The mean mutation frequency ±standard deviation of two to nine independent cultures is shown. Cells containing the parental hybrid pMLH1_h(41-86) exhibited a mutation frequency of 2.3×10⁻⁴. The mutation defect (shown above each bar) for each variant and control was calculated by dividing the mutation frequency of cells expressing the variant by the mutation frequency of cells expressing parental hybrid pMLH1_h(41-86). MLH1_h(41-86) genes containing spontaneous nucleotide deletions in codon 43 (an A-deletion) and 45 (a CA-deletion) are referred to as “frameshift-1” and “−2”, respectively.

### DETAILED DESCRIPTION OF THE INVENTION

As mentioned, the invention includes methods for the use of protein sequences (and the gene sequences encoding the proteins) to diagnose an individual's susceptibility to develop cancer as compared to a normal individual (or that same individual's risk if they carried two wild-type copies of the mismatch repair gene). A “normal” individual is a human subject that carries two copies of the wild-type DNA mismatch repair gene or carries one copy of the wild-type gene and one copy of a known silent polymorphism. Cancer susceptibility is defined as the lifetime risk to develop cancer and may be based in part on age, sex, ethnicity, environmental factors, and genetic risk factors.

A method is described herein for the prospective identification of DNA mismatch repair proteins having an amino acid substitution which impairs DNA mismatch repair. The method includes the steps of generating DNA mismatch repair genes with random sequence alterations, introducing these genes into appropriate host cells, functionally analyzing these genes in vivo, identifying any inactivating alterations, and making a quantitative assessment of the level to which DNA mismatch repair is effected thereby. This method involves the use of a new DNA molecule having an in-frame microsatellite tract in the native yeast ADE2 gene (ADE2::MS3::ADE2 allele). The method also includes yeast strains which carry the ADE2::MS3::ADE2 allele and are deficient in MMR gene function via specific deletions of a native DNA mismatch repair gene. As described below (in Examples 6, 7 and 8) the method provides a basis for the direct visual assessment of DNA mismatch repair function based on the examination of the color of yeast colonies.

A method has been described previously in the aforementioned patent application WO 02/081624 A3 for the analysis of human missense alterations using a DNA molecule encoding a yeast protein involved in DNA mismatch repair in which a portion of the coding sequence has been replaced with the homologous coding sequence of the human orthologue to produce a hybrid human-yeast gene that retains function in DNA mismatch repair in vivo. In contrast, the present method features the use of new DNA molecules which encode additional portions of human DNA mismatch repair genes. Specifically, yeast proteins containing portions of human MLH1 amino acids 175-341 and human MSH2 amino acids 621-862 are described and shown to retain function for DNA mismatch repair in yeast cells deficient in the corresponding native DNA mismatch repair gene. Also, a method for the use of the new human-yeast hybrids to examine human missense alterations is disclosed herein.

In one embodiment of the method of this invention, human-yeast hybrid genes having random sequence alterations are tested in a prospective screen to identify novel human missense alterations which impair MMR gene function. In another embodiment of the method, previously observed human gene alterations which confer an uncertain functional significance are recapitulated in the human-yeast hybrids and tested for their effects on gene function.

The present disclosure details the use the aforementioned methods, as well as methods described previously (see WO 02/081624 A3), to generate and determine the function of some 180 DNA mismatch repair proteins, each one having one amino acid substitution. The 180 variants are classified according to those that confer upon an individual a greater than normal susceptibility to develop cancer or, alternatively, no greater than normal susceptibility to develop cancer (see Table 1).

An important feature of this invention is a method for the diagnosis of susceptibility to cancer development based on the sequence of an individual's DNA mismatch repair gene and the known functional consequence of any alteration on DNA mismatch repair. The methods of the invention provide an approach for classifying amino acid substitutions by the degree of risk they confer, because the methods described permit a quantitative measure of DNA mismatch repair function. Thus, amino acid substitutions (or missense changes in the nucleic acid sequence) are classified as “silent polymorphisms”, i.e., conferring upon an individual no greater susceptibility to develop cancer compared to a normal individual, “efficiency polymorphisms”, i.e., conferring upon an individual a greater than normal susceptibility to develop cancer (and which can also be characterized as a “medium” risk), and “inactivating mutations”, i.e., conferring upon an individual a relatively high susceptibility to develop cancer compared to a normal individual. The methods of this invention can be used in a diagnostic test setting to evaluate predisposition to the onset of cancer in a human subject and to classify that individual's risk compared to a normal individual.

Another feature of this invention encompasses any of the aforementioned methods for analysis of, but not limited to, variants of the hMSH2, hMSH3, hMSH4, hMSH6, hMLH1, hMLH3, hPMS1 and hPMS2 genes.

In addition to new technology and methods described below, the investigations leading to the present invention use technology and methods described previously (WO 02/081624 A3). A quantitative in vivo assay of DNA mismatch repair was developed in the lower eukaryote Saccharomyces cerevisiae (a yeast) and this technology was shown to be capable of distinguishing DNA mismatch repair proteins containing silent amino acid substitutions from those containing “mutations” (i.e., functionally inactivating substitutions). Here, the yeast system has been adapted and extended determine the functional consequence of additional amino acid substitutions. The information generated with this technology will be useful for unambiguous genetic testing for HNPCC. The methods described demonstrate the usefulness of measuring the function of MMR proteins in vivo. The invention disclosed here further demonstrates the existence of a novel class of amino acid substitutions that result in proteins which are functional in MMR, but which impair efficiency relative to the native protein. This class of variant MMR proteins is referred to as “efficiency polymorphisms”. Some of these amino acid substitutions have been observed in individuals with “sporadic” (i.e., non-familial) colorectal cancer, suggesting that individuals in the general human population may indeed have different efficiencies of DNA mismatch repair due to common polymorphisms. The efficiency polymorphisms discovered with this invention, as well as those that can be identified in the future using this invention, are predictive of individual differences in susceptibility to develop cancer. Individuals in the general population may thus be screened for cancer susceptibility as a result.

In the described study delineated further herein, missense codons previously observed in human genes were introduced at the homologous residue in the yeast MLH1 (SEQ ID NO: 29) or MSH2 (SEQ ID NO: 203) genes. In addition, genes which encode functional hybrid human-yeast MLH1 and MSH2 proteins have been constructed, and they have been used to evaluate missense codons at positions which are not conserved between yeast and humans. Three classes of missense codons have thus been found: (1) complete loss-of-function, i.e. mutations; (2) variants indistinguishable from wild-type protein, i.e. silent polymorphisms; and (3) functional variants which support MMR at reduced efficiency i.e. efficiency polymorphisms. There is a good correlation between the functional results in yeast and available human clinical data regarding penetrance of the missense codon. The discovery of efficiency polymorphisms, some of which did not appear to be associated with HNPCC, raises the possibility that differences in the efficiency of DNA mismatch repair exist between individuals in the human population due to common polymorphisms, and that such polymorphisms predispose to early onset of cancer development.

In brief, the present invention provides a diagnostic approach for diseases, such as HNPCC, that are associated with defects in MMR and provides a method for determining whether any specific genetic sequence of a gene associated with MMR that differs from a consensus sequence is a mutation (i.e., non-functional protein), a silent polymorphism (i.e., normal protein function) or an efficiency polymorphism (i.e., functional protein with reduced efficiency in MMR). The invention enables the generation of databases of the functional significance of specific amino acid substitutions on MMR protein function in vivo. Such databases will allow accurate and unambiguous interpretation of genetic tests of MMR.

A novel prospective screen for the identification of novel inactivating amino acid substitutions in DNA mismatch repair proteins is described. In brief, the screen is based on the random mutagenesis of a test sequence and the expression of that sequence in a yeast host strain lacking the corresponding native gene. If the mutagenized gene complements the MMR deficiency of the host strain, individual yeast colonies will appear white. If the mutagenized gene does not complement the MMR deficiency, i.e., a mutant, the yeast colonies will appear white with red sectors. Thus, colonies with a mutant MMR gene can be rapidly identified by visual inspection. These colonies are then used as the starting material to retrieve the test sequence and identify the genetic alteration causing loss-of-MMR function. These sequences are then used to diagnose an individual as having an increased risk to develop cancer.

The invention reports the function of MLH1 proteins having all possible amino acid substitutions at residues 43 and 44. These variant proteins were tested in quantitative in vivo MMR assays which allowed the classification of each as having either a mutation, a silent polymorphism, or an efficiency polymorphism. Use of sequences and functional information thus obtained represents an additional approach for the diagnosis of an individual as having an increased risk to develop cancer.

In this invention, the test genetic sequence can be a yeast orthologue variant of the human gene sequence or a human-yeast hybrid sequence of said variant. Illustratively, the human gene involved in DNA mismatch repair can be selected from the group consisting of the hMSH2 (SEQ ID NO: 205), hMSH3 (SEQ ID NO: 41), hMSH4 (SEQ ID NO: 42), hMSH6 (SEQ ID NO: 43), hMLH1 (SEQ ID NO: 31), hMLH3 (SEQ ID NO: 44), hPMS1 (SEQ ID NO: 45) and hPMS2 (SEQ ID NO: 46) genes, and especially, the hMLH1 and hMSH2 genes.

It is anticipated that the methods described in this text will be incorporated into genetic testing for cancer diagnosis and predisposition in a variety of ways. These uses fall into two general types which are best illustrated by way of examples, as follows: First, the methods are used to produce a database of functional information which is used as a reference source. Following the sequencing of an individual's MMR gene(s) and finding of a variant of uncertain significance (e.g. single amino acid substitution, small in-frame deletion or insertion), the function of that variant will be interpreted by comparison to the information in the database. If the observed variant appears in the database as one which confers a complete or partial loss-of-MMR function the alteration is considered pathogenic and a cause of increased susceptibility to cancer. If the observed variant is classified as a functionally silent polymorphism the alteration is considered non-pathogenic. In an index patient (i.e. a patient with an existing cancer) this information would be of value for disease prognosis and predicting the response to certain therapies, which would play a vital role in management of that patient. The ability to classify variants of uncertain significance would provide the information needed to identify family members of the index patient who would benefit from preventative cancer screening. For individuals who carry a pathogenic variant but with no detectable cancer, a likely recommendation might be to increase the frequency of cancer surveillance in them and, perhaps, to begin cancer prevention strategies. On the other hand, individuals who do not carry a pathogenic variant might be able to follow a more routine plan of cancer avoidance.

Another use of the methods described in this text would be the development of a standardized genetic test whereby an individual would be screened for all, or a subset of, the variants for which function in MMR is known. This could be accomplished by development of a genotyping assay based on either commercially-available or new technologies. These technologies may include, but are not limited to, those based on DNA:DNA hybridization, DNA:RNA hybridization, “genechip” analysis, PCR, DNA sequencing, primer extension, etc. They might also include screens based on the differential screening of an individual's MMR proteins. These technologies may include, but are not limited to, those that would be based on variant-specific antibodies (e.g. Western blots, radioimmunoassays, immunohistochemistry) or direct protein sequencing. In general, the basis of these technologies is to test for the presence of pre-determined sequence variations in a biological sample using a universally-formatted assay. The assay would determine whether the individual's genotype or protein profile matched a result that would indicate they are a carrier of a MMR gene mutation and thus have a high risk to develop cancer. For example, the assay would reveal the presence of any missense mutations that were classified (using the methods described in this patent application) as a pathogenic mutation. Depending on the results of this test, an individual would be prescribed specific treatments or regimes for cancer surveillance appropriate for the individual's MMR status. Finally, in considering the utility of this invention, it is important to note that the aforementioned applications would not be possible without the methods described herein to ascertain a function for variants of uncertain significance.

### DESCRIPTION OF SPECIFIC EMBODIMENTS

The invention is further illustrated by way of the following examples, which are not intended to be limiting.

### Example 1

### Functional Analysis of MLH1 Variants

Rationale. Sequencing of the human MLH1 gene (SEQ ID NO: 31) from many individuals has revealed over 100 different nucleotide variations (i.e. missense codons) that are predicted to give rise to a protein with single amino acid substitutions compared to the wild-type human MLH1 protein (SEQ ID NO: 32). In the absence of additional information these alleles are often termed “variants of uncertain significance” because the functional consequence of the substitutions is unclear. Gaining an understanding of the consequence of these substitutions is critical in light of the known relationship between MMR activity and predisposition to develop certain types of cancer. Taking advantage of the high level of amino acid conservation between the human and yeast S. cerevisiae MMR proteins, a standardized in vivo assay of MMR function in yeast to quantitatively assess the functional significance of missense codons in MMR genes was developed previously (25-27). Using that yeast-based assay, in the present example 21 known human MLH1 variants were analyzed for their effect on MMR activity. These variants, which can be viewed as having been of uncertain significance prior to now, have been previously reported in the literature (28-32) or public databases maintained on the internet by the ICG-HNPCC (http://www.nfdhtl.nl), Human Gene Mutation Database (http://www.hgmd.org) or GeneSnP Database (http://www.genome.utah.edu/genesnps/). Assay materials and procedures are described in detail below, together with the results.

Plasmids. Plasmid pMETc (p413MET25, (33)) contains a HIS3 selectable marker, a centromere sequence (CEN6) for mitotic stability, an ARS4 origin of DNA replication, the ampicillin-resistance gene for positive selection in E. coli, and a multicloning site between the MET25 promoter and CYC1 terminator. Plasmid pMLH1, a derivative of pMETc lacking the MET25 promoter, contains a 3.8-kb genomic DNA fragment from S. cerevisiae strain S288C including the MLH1 gene coding sequence and 1.5-kb of 5′ flanking sequence (26). Plasmid pSH91 contains a TRP1 selectable marker, a centromere sequence (CEN11), an ARS1 origin of replication, the ampicillin resistance gene, and the URA3 coding sequence preceded by an in-frame (GT)₁₆G tract (34).

Mutations (n=21) were introduced into the yeast MLH1 gene (SEQ ID NO: 29) using the QuikChange Site-Directed Mutagenesis kit (Stratagene, La Jolla, Calif.) following the manufacturer's instructions. Plasmid pMLH1 was used as template for the following variants (yeast alterations given): G19A (SEQ ID NO: 578), E20D (SEQ ID NO: 129), G64W (SEQ ID NO: 130), C74Y (SEQ ID NO: 131), F77V (SEQ ID NO: 196), R97P (SEQ ID NO: 132), E99D (SEQ ID NO: 133), P138R (SEQ ID NO: 579), R179G (SEQ ID NO: 134), S190P (SEQ ID NO: 135), L272V (SEQ ID NO: 136), K286Q (SEQ ID NO: 580), D304V (SEQ ID NO: 581), A444T (SEQ ID NO: 137), Q552P (SEQ ID NO: 138), R559P (SEQ ID NO: 139), P653S (SEQ ID NO: 582), P661L (SEQ ID NO: 197), R672Q (SEQ ID NO: 140), E676G (SEQ ID NO: 141), and R768S (SEQ ID NO: 142). Sense and antisense oligonucleotide primers were obtained from a commercial source (BioSynthesis Inc. Lewisville, Tex.) and, to facilitate screening for mutant clones, included a silent restriction site change in addition to the desired missense alteration (Table 2 and 6a). For all mutations at least three independent clones were tested for function in yeast with identical results. At least one clone that contained the appropriate restriction site alteration was sequenced on both the coding and non-coding DNA strands to confirm the sequence and verify the native sequence over at least 100 bp on either side of the introduced mutation. The data presented below are derived from four replicate cultures of a single mutant clone that had been confirmed by DNA sequence analysis.

Yeast strains and media. The strains used in this invention were derived from S. cerevisiae YPH500 (MATα ade2-101 his3-Δ200 leu2-Δ1 lys2-801 trp1-Δ63 ura3-52) (35). Strain YBT24 contains a deletion of the entire MLH1 coding sequence and has the genotype MATα ade2-101 his3-Δ200 leu2-Δ1 lys2-801 trp1-Δ63 ura3-52 mlh1Δ::LEU2 (26). Yeast strains were maintained in SD medium (0.67% yeast nitrogen base without amino acids, 2% dextrose) containing the appropriate growth supplements. Yeast strains were transformed with plasmid DNAs using the polyethylene glycol-lithium acetate method (36).

Quantitative in vivo MMR assay. Standardized M assays based on mutation to ura3 FOA^{R} were performed as described previously (25, 26). Briefly, YBT24 transformants containing an MLH1 expression vector and pSH91 were cultured overnight in medium lacking uracil and subcultured in liquid media containing adenine, lysine, and uracil, which allows growth of ura3 FOA^{r} mutants [which arise as a result of slippage in the (GT)₁₆G-tract]. After 24 hours in culture, OD₅₉₅ measurements were taken and an aliquot was plated on SD plates containing adenine, lysine, uracil and FOA (1 mg/ml). Mutation frequencies were calculated as described previously (26), except that the concentration (CFU/ml) of total cells was determined from OD₅₉₅ readings using the determined value 1 OD₅₉₅=1.1×10⁷ CFU/ml. The mutation defect is defined as the ratio of the mutation frequency in the test strain divided by that observed in the appropriate MMR-proficient control strain.

Statistical Comparisons. Mean mutation frequencies (n=4) from independent experiments were compared to control values within each particular experiment using T-tests (Excel 97, Microsoft). The Bonferroni adjustment was used to set the significance level at P≦0.025 to reject the null hypothesis (37, 38). Standard deviations and 95% confidence intervals (CI) were calculated using Excel.

Results. Site-directed mutations were made in plasmid pMLH1 to generate missense codons in the yeast MLH1 gene (SEQ ID NO: 29). These missense codons alter the yeast MLH1 coding sequence (SEQ ID NO: 30) to encode a protein with amino acid substitutions identical to those previously observed in the human population (Table 2 and 6a). The variant MLH1 genes and control plasmids pMLH1 and pMETc were introduced into YBT24 containing pSH91 and tested for activity in the standardized MMR assay. Representative yeast strains were assayed in 6 independent experiments and the results are listed in Table 3. Strain YBT24 containing pMLH1 exhibited a mean mutation frequency of 1.4×10⁻⁵. The same strain containing the pMETc expression vector, which lacks an MLH1 gene, exhibited a mean mutation frequency of 265×10⁻⁵. These results indicate that, depending on the experiment, YBT24 deficient in MLH1 exhibits a mutation defect ranging from 136 to 241. Yeast strains expressing MLH1p with the amino acid substitutions G64W (SEQ ID NO: 130), S190P (SEQ ID NO: 135), D304V (SEQ ID NO: 581), and R768S (SEQ ID NO: 142) exhibited mean mutation frequencies of 171-445×10⁻⁵ (Table 3). These mutation frequencies represent mutation defects of 122-318. Statistical analyses of the mutation frequencies determined in each experiment showed that clones containing MLH1 G64W, S190P, D304V, and R768S were statistically greater than the strain expressing wild-type yeast MLH1p. Moreover, the mutation frequencies were greater than or not significantly different from that exhibited by YBT24 containing pMETc. These results demonstrate that amino acid substitutions G64W, S190P, D304V, and R768S result in complete loss-of-MMR function. Therefore, these four alterations (G64W, S190P, D304V, and R768S) are considered inactivating mutations.

Strain YBT24 expressing the G19A (SEQ ID NO: 578), P138R (SEQ ID NO: 579), L272V (SEQ ID NO: 136), K286Q (SEQ ID NO: 580), A444T (SEQ ID NO: 137), P661L (SEQ ID NO: 197), and R672Q (SEQ ID NO: 140) variants exhibited mean mutation frequencies of 0.7-1.8×10⁻⁵ (Table 3), levels which were not significantly different from the mutation frequency exhibited by YBT24 expressing the wild-type yeast MLH1 gene. These results demonstrate that the G19A, P138R, L272V, K286Q, A444T, P661L, and R672Q amino acid substitutions do not detectably alter MLH1p function in MMR. Therefore these seven alterations (G19A, P138R, L272V, K286Q, A444T, P661L and R672Q) are considered silent polymorphisms.

Ten of the codon alterations in MLH1 gave rise to proteins which exhibited intermediate levels of MMR activity. The E20D (SEQ ID NO: 129), C74Y (SEQ ID NO: 131), F77V (SEQ ID NO: 196), R97P (SEQ ID NO: 132), E99D (SEQ ID NO: 133), R179G (SEQ ID NO: 134), Q552P (SEQ ID NO: 138), L559P (SEQ ID NO: 139), P653S (SEQ ID NO: 582), and E676G (SEQ ID NO: 141) variants exhibited mean mutation frequencies of 1.9 to 95×10⁻⁵ (Table 3). Statistical analysis of the independent experiments showed that the mutation frequencies were significantly different from that exhibited by YBT24 containing either pMLH1 or pMETc. The results indicate that the E20D, C74Y, F77V, R97P, E99D, R179G, Q552P, L559P, P653S, and E676G variants confer mutation defects of 68, 59, 56, 120, 7.7, 1.4, 23, 29, 11 and 5.1, respectively. These alterations are considered efficiency polymorphisms (ΔE) because they confer a reduced, but not complete, loss-of-MMR function (i.e. partial function in MMR). The corresponding amino acid alterations in the human MLH1 protein (see Tables 2 and 6a) are considered to have an equivalent effect on MMR activity.

### Example 2

### Construction and Functional Analysis of Hybrid Human-Yeast MLH1 Genes

Rationale. Approximately 47% of the MLH1 nucleotide alterations observed in the human population are predicted to alter an amino acid residue which is not conserved in the yeast MLH1p. To address this issue a series of hybrid human-yeast genes that contained portions of human MLH1p spanning amino acids 1-177 (of 756 total) were developed and shown to confer moderate levels of MMR activity (27). In this invention, the development of six new human-yeast hybrid genes that contain regions of human MLH1p (spanning amino acids 175-341) replacing the homologous region of yeast MLH1p are reported. Except for the noted chimeric region, the structure of each hybrid gene is identical to the parental expression vector pMLH1 (see Example 1), which contains the native yeast MLH1 gene and 5′ regulatory region.

Plasmids. Hybrid human-yeast MLH1 genes were constructed using pMLH1 (see Example 1) as the parental vector. MLH1_h(175-267). This hybrid gene was constructed using a three-piece overlap extension polymerase chain reaction (PCR) method. A 179-bp fragment of the human MLH1 coding sequence was amplified by PCR from a commercially-available cDNA clone (ATCC#217884, American Type Culture Collection, Rockville, Md.) using primers SEQ ID NO: 33 and SEQ ID NO: 34. A 465-bp fragment from the 5′ end of yeast MLH1 was amplified from S. cerevisiae strain S288C genomic DNA using primers SEQ ID NO: 35 and SEQ ID NO: 36. A 1535-bp fragment from the 3′ end of yeast MLH1 was amplified from S. cerevisiae strain S288C genomic DNA using primers SEQ ID NO: 37 and SEQ ID NO: 38. All PCR amplifications were carried out using Pfu DNA polymerase (Stratagene, La Jolla, Calif.) using the manufacturer's recommended conditions. The three fragments were mixed in approximately equimolar amounts and subjected to overlap extension PCR using primers SEQ ID NO: 35 and SEQ ID NO: 39. The overlap extension PCR product was digested with AatII and Bsu36I and ligated into AatII-Bsu36I digested yeast MLH1 expression vector pMLH1 (27). The protein encoded by this gene contains amino acids 1-171 and 268-769 of yeast MLH1p and amino acids 175-267 of the human MLH1p (SEQ ID NO: 40). MLH1_h(175-214). An approximately 900-bp fragment of yeast MLH1 was amplified from S. cerevisiae strain S288C genomic DNA using the primers SEQ ID NO: 160 and SEQ ID NO: 161. The fragment was digested with BtgI and Bsu36I and ligated into BtgI-Bsu36I digested pMLH1_h(175-267), replacing the equivalent portion of the human-yeast hybrid sequence. The protein encoded by this gene contains amino acids 1-171 and 212-769 of yeast MLH1p and amino acids 175-214 of the human MLH1p (SEQ ID NO: 198). MLH1_h(208-267). An approximately 560-bp fragment of yeast MLH1 was amplified from S. cerevisiae strain S288C genomic DNA using the primers SEQ ID NO: 35 and SEQ ID NO: 162. The fragment was blunt-end cloned into EcoRV-digested plasmid pBluescript II (KS-) (Stratagene, La Jolla Calif.). The cloned yeast fragment was then excised using an AatII-BtgI double digest and ligated into AatII-BtgI digested pMLH1_h(175-267), replacing the equivalent portion of the human-yeast hybrid sequence. The protein encoded by this gene contains amino acids 1-204 and 268-769 of yeast MLH1p and amino acids 208-267 of the human MLH1p (SEQ ID NO: 199). MLH1_h(265-341). This human-yeast hybrid gene was constructed using a two-piece overlap extension PCR method. A 255-bp fragment of the human MLH1 coding sequence was amplified by PCR from ATCC cDNA clone #217884 (American Type Culture Collection, Rockville, Md.) using primers SEQ ID NO: 163 and SEQ ID NO: 164. A 495-bp fragment from the central portion of yeast MLH1 was amplified from S. cerevisiae strain S288C genomic DNA using primers SEQ ID NO: 165 and SEQ ID NO: 161. PCR amplifications were carried out using Pfu DNA polymerase (Stratagene, La Jolla Calif.) using the manufacturer's recommended conditions. The two fragments were mixed in approximately equimolar amounts and subjected to overlap extension PCR using primers SEQ ID NO: 163 and SEQ ID NO: 161. The overlap extension PCR product was digested with SpeI and ligated into SpeI digested expression vector pMLH1 (27), replacing the equivalent portion of the yeast gene. The correct orientation of the insert was verified by restriction fragment length polymorphism (RFLP) analysis using an introduced SalI site in the primer SEQ ID NO: 163. The protein encoded by this gene contains amino acids 1-264 and 342-769 of yeast MLH1p and amino acids 265-341 of the human MLH1p (SEQ ID NO: 200). MLH1_h(265-311). An approximately 620-bp fragment of yeast MLH1 was amplified from S. cerevisiae strain S288C genomic DNA using the primers SEQ ID NO: 166 and SEQ ID NO: 161. The fragment was digested with AccB7I and Bsu36I and ligated into AccB7I-Bsu36I digested pMLH1_h(265-341), replacing the equivalent portion of the human-yeast hybrid sequence. The protein encoded by this gene contains amino acids 1-264 and 312-769 of yeast MLH1p and amino acids 265-311 of the human MLH1p (SEQ ID NO: 201). MLH1_h(298-341). An approximately 840-bp fragment of yeast MLH1 was amplified from S. cerevisiae strain S288C genomic DNA using the primers SEQ ID NO: 35 and SEQ ID NO: 167. The fragment was digested with ClaI and AccB7I and ligated into ClaI-AccB7I digested pMLH1_h(265-341), replacing the equivalent portion of the human-yeast hybrid sequence. The protein encoded by this gene contains amino acids 1-297 and 342-769 of yeast MLH1p and amino acids 298-341 of the human MLH1p (SEQ ID NO: 202). All hybrid MLH1 genes were verified by DNA sequencing.

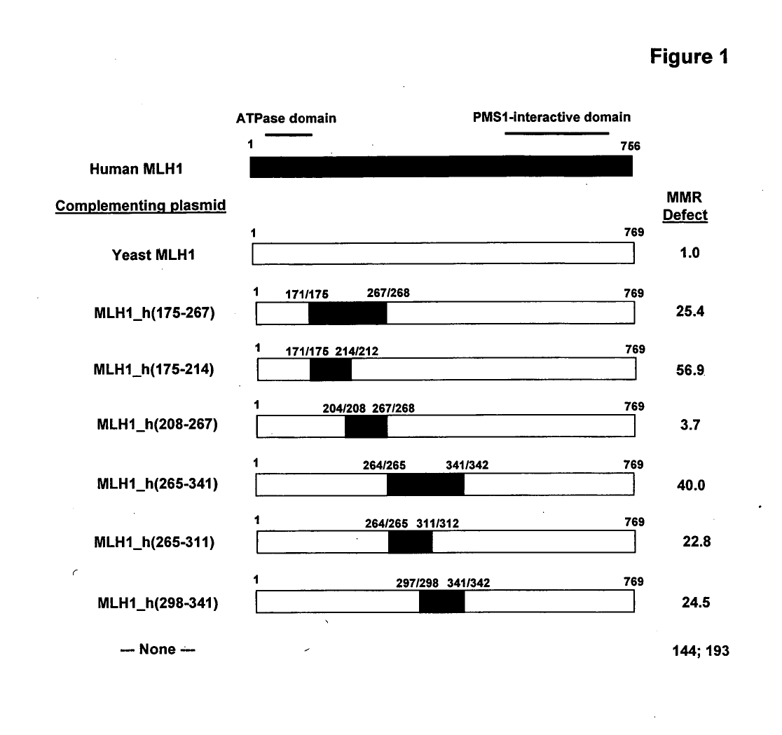

Results. Six hybrid human-yeast MLH1 genes were constructed by replacing a region of the yeast MLH1 coding sequence with the homologous region of the human MLH1 (FIG. 1). Plasmids carrying the human-yeast hybrid MLH1 genes were introduced into yeast strain YBT24 containing pSH91 and standardized MMR assays were carried out as described previously (see Example 1). Representative strains were assayed in independent experiments and the results are shown in Table 4. Strain YBT24 containing pMETc, which lacks an MLH1 gene, exhibited mutation frequencies of 174-303×10⁻⁵ while the same strain containing pMLH1 exhibited mutation frequencies of 1.2-1.6×10⁻⁵. These results represent mutation defects in the range 144-193 for cells lacking functional MLH1p. The mutation frequencies of YBT24 containing the hybrid MLH1 genes MLH1_h(175-267), MLH1_h(175-214), MLH1_h(208-267), MLH1_h(265-341), MLH1_h(265-311), and MLH1_h(298-341), were 30.8×10⁻⁵, 89.4×10⁻⁵, 5.7×10⁻⁵, 48.3×10⁻⁵, 35.7×10⁻⁵, and 38.4×10⁻⁵, respectively. These levels represent mutation defects ranging from 3.7 to 56.9 (Table 4). Although the hybrid genes did not appear to fully complement the MMR defect of YBT24, the results show that each hybrid was partially functional in MMR. The availability of these human yeast hybrid genes increase the number of human codon alterations which can be functionally evaluated in yeast.

### Example 3

### Functional Analysis of MLH1p Variants Using Human-Yeast Hybrid Genes

Plasmids. Hybrid human-yeast MLH1 expression vectors pMLH1_h(1-86), pMLH1_h(41-86), pMLH1_h(77-134), and pMLH1_h(77-177) have been described previously (26, 27). The indicated alterations were made in the humanized region of these plasmids using the QuikChange Mutagenesis kit (Stratagene) and the oligonucleotides shown in Table 2. Hybrids MLH1_(41-86) containing the G67E alteration and MLH1_h(77-134) containing the N35S (equivalent to human MLH1 N38S) and C77R alterations were identified in the prospective genetic screen (Example 8). At least one clone that contained the appropriate restriction site alteration was sequenced on both the coding and non-coding DNA strands to confirm the sequence and verify the native sequence over at least 100 bp on either side of the introduced mutation. The data presented below are derived from four replicate cultures of a single mutant clone that had been confirmed by DNA sequence analysis.

Results. Hybrid human-yeast MLH1 genes containing the indicated alteration were transformed into YBT24 containing pSH91 and assayed for MMR activity as described in Example 1. Mutation frequencies were compared to YBT24 harboring the parental hybrid MLH1 gene, and pMETc, which lacks an MLH1 gene. Mean mutation frequencies (n=4) were compared to that exhibited by control strains using T-tests with significance levels of P≦0.025 (Example 1). As shown in Table 5 (“Experiment #1”), the mutation frequencies conferred by A29S and 132V substitutions in hybrid MLH1_h(1-86), were 33.0×10⁻⁵ and 32.6×10⁻⁵, respectively. These levels were not significantly different from the mutation frequency conferred by the parental hybrid MLH1_h(1-86) (23.3×10⁻⁵). The mutation frequency conferred by the G67E substitution in hybrid MLH1_h(41-86) was 153×10⁻⁵ (Table 5, “Experiment #2”). This level was significantly greater than that conferred by the parental hybrid MLH1_h(41-86) (27.8×10⁻⁵) and significantly less than that conferred by pMETc (234×10⁻⁵). The mutation frequencies conferred by N35S and C77R substitutions in hybrid MLH1_h(77-134), were 214×10⁻⁵ and 290×10⁻⁵, respectively (Table 5, “Experiment #3”). These levels were significantly greater than the mutation frequency conferred by the parental hybrid MLH1_h(77-134) (11.5×10⁻⁵). Moreover, the mutation frequencies conferred by N35S and C77R were greater than or not statistically different from that conferred by pMETc (182×10⁻⁵), indicating that they confer a complete loss-of-MMR function. The mutation frequencies conferred by A128P and A160V substitutions in hybrid MLH1_h(77-177), were 228×10⁻⁵ and 5.9×10⁻⁵, respectively (Table 5, “Experiment #4”). For the A128P substitution the mutation frequency was significantly greater than the mutation frequency conferred by the parental hybrid MLH1_h(77-177) (6.6×10⁻⁵) and significantly less than that conferred by pMETc. For the A160V substitution the mutation frequency was not statistically different from that conferred by the parental hybrid MLH1_h(77-177).

In summary, the results indicate that the N35S and C77R substations confer a complete loss-of-MMR function. Thus, these two alterations (N35S and C77R) are inactivating mutations. The A29S, I32V and A160V substitutions do not effect MMR function and are considered silent polymorphisms. The G67E and A128P substitutions confer intermediate levels of MMR activity and are considered efficiency polymorphisms.

### Example 4

### Functional Analysis of MSH2p Variants

Rationale. Sequencing of the human MSH2 gene (SEQ ID NO: 205) from many individuals has revealed over 100 different nucleotide variations (i.e. missense codons) that are predicted to give rise to a protein with single amino acid substitutions compared to the wild-type human MLH1 protein (SEQ ID NO: 206). These variants, which can be viewed as having been of uncertain significance prior to now, have been previously reported in the literature (29, 39-47) or public databases maintained on the internet by the ICG-HNPCC (http://www.nfdhtl.nl), Human Gene Mutation Database (http://www.hgmnd.org), the Swiss Protein Database (http://us.expasy.org) and the single nucleotide polymorphism (SNP) databases (http://dir-apps.niehs.nih.gov/egsnp/). Taking advantage of the high level of amino acid conservation between the human and yeast S. cerevisiae MMR proteins, a standardized in vivo assay of MMR function in yeast to quantitatively assess the functional significance of missense codons in MMR genes was developed previously (25-27). Using that yeast-based assay, in the present example 41 known human MSH2 variants were analyzed for their effect on MMR activity. Assay materials and procedures are described in detail below, together with the results.

Plasmids. Plasmids pMETc, the parental expression vector lacking a cDNA insert, and pSH91, which contains the URA3 coding sequence preceded by an in-frame (GT)₁₆G tract, were described in Example 1. Plasmid pMETc/MSH2 contains the 2.9-kb MSH2 coding sequence from S. cerevisiae strain S288C cloned between the MET25 promoter and CYC1 terminator of pMETc (25, 26).

Mutations (n=41) were introduced into the yeast MSH2 gene (SEQ ID NO: 203) using the QuikChange Site-Directed Mutagenesis kit (Stratagene, La Jolla, Calif.) following the manufacturer's instructions. Plasmid pMETc/MSH2 was used as template for the following variants (yeast alterations given): P30L (SEQ ID NO: 583), T44M (SEQ ID NO: 532), Q61P (SEQ ID NO: 207), VE106/107-del (SEQ ID NO: 584), N123S (SEQ ID NO: 208), D163H (SEQ ID NO: 209), N182S (SEQ ID NO: 75), E194G (SEQ ID NO: 210), C195R (SEQ ID NO: 211), C195W (SEQ ID NO: 585), A267V (SEQ ID NO: 212), G317V (SEQ ID NO: 586), S318C (SEQ ID NO: 533), C345R (SEQ ID NO: 76), C345Y (SEQ ID NO: 77), G350R (SEQ ID NO: 587), P361L (SEQ ID NO: 588), L402F (SEQ ID NO: 213), L402V (SEQ ID NO: 214), P456-del (SEQ ID NO: 589), L457P (SEQ ID NO: 590), L521P (SEQ ID NO: 215), R552C (SEQ ID NO: 591), E580V (SEQ ID NO: 592), N601S (SEQ ID NO: 593), L613R (SEQ ID NO: 594), D621N (SEQ ID NO: 595), A627V (SEQ ID NO: 78), P640T (SEQ ID NO: 596), H658R (SEQ ID NO: 79), G702R (SEQ ID NO: 216), G702V (SEQ ID NO: 217), M707I (SEQ ID NO: 218), I710T (SEQ ID NO: 80), G711R (SEQ ID NO: 81), C716R (SEQ ID NO: 82), V741I (SEQ ID NO: 597), I754V (SEQ ID NO: 219), G770R (SEQ ID NO: 83), I789V (SEQ ID NO: 84), and K873E (SEQ ID NO: 220). Sense and antisense oligonucleotide primers were obtained from a commercial source (BioSynthesis Inc. Lewisville, Tex.) and, to facilitate screening for mutant clones, included a silent restriction site change in addition to the desired missense alteration (Tables 6 and 6a). For all mutations at least three independent clones were tested for function in yeast with identical results. At least one clone that contained the appropriate restriction site alteration was sequenced on both the coding and non-coding DNA strands to confirm the sequence and verify the native sequence over at least 100 bp on either side of the introduced mutation. The data presented below are derived from four replicate cultures of a single mutant clone that had been confirmed by DNA sequence analysis.

Yeast strains and media. The strains used in this invention were derived from S. cerevisiae YPH500 (MATα ade2-101 his3-Δ200 leu2-Δ1 lys2-801 trp1-Δ63 ura3-52) (35). Strain YBT25 contains a deletion of the entire MSH2 coding sequence and has the genotype MATα ade2-101 his3-Δ200 leu2-Δ1 lys2-801 trp1-Δ63 ura3-52 msh2Δ::LEU2 (26). Strains were maintained in SD medium (0.67% yeast nitrogen base without amino acids, 2% dextrose) containing the appropriate growth supplements. Strains were transformed with plasmid DNAs using the polyethylene glycol-lithium acetate method (36).

Quantitative in vivo MMR assays. The standardized in vivo MMR assay based on instability of the (GT)₁₆G::URA3 allele in pSH91 was described in Example 1. Mean mutation frequencies from 4 replicate cultures are reported. Statistical comparisons were carried out as described in Example 1 with conclusions based on results within each independent experiment. Forward mutation rates to canavanine resistance were determined by fluctuation analysis using the method of the median (48). Individual colonies (YBT25 transformed with the MSH2 expression vectors) were expanded in liquid SD media containing the appropriate supplements. After 24 hours in culture, OD₅₉₅ measurements were taken and an aliquot was plated on SD plates containing the appropriate supplements and 60 μg/ml canavanine. Canavanine-resistant colonies were counted after 2-3 days growth at 30° C. Mutation frequencies were determined by dividing the concentration (CFU/ml) of canavanine resistant colonies by the concentration (CFU/ml) of total cells. Median mutation frequencies and 95% confidence intervals (CI) were calculated using Microsoft Excel 97. The mutation defect is defined as the ratio of the mutation frequency in the test strain divided by that observed in the appropriate MMR-proficient control strain.

Results. Site-directed mutations were made in plasmid pMETc/MSH2 to generate missense codons in the yeast MSH2 gene (SEQ ID NO: 203). These missense codons alter the yeast MSH2 coding sequence (SEQ ID NO: 204) to encode a protein with amino acid substitutions identical to those previously observed in the human population (Table 6 and 6a). The variant MSH2 genes and control plasmids pMETc/MSH2 and pMETc were transformed into YBT25 containing pSH91 and tested for activity in both the standardized MMR assay based on GT-tract stability (Example 1) and a fluctuation test for canavanine resistance, which detects predominantly base substations and frameshift mutations in mononucleotide tracts in the arginine permease (CAN1) gene (49). Representative yeast strains were assayed in independent experiments and the results are summarized in Table 7.

As measured using the (GT)₁₆G::URA3 allele, strain YBT25 containing the pMETc expression vector, which lacks an MSH2 gene, exhibited a mean mutation frequency of 350×10⁻⁵ (Table 7, “None”). The same strain containing the pMETc/MSH2 expression vector exhibited a mean mutation frequency of 4.0×10⁻⁵ (Table 7, “MSH2”). These results show that expression of wild-type yeast MSH2 from pMETc/MSH2 complements the MSH2-deficiency of YBT25 and indicates that YBT25 lacking MSH2p has a mutation defect of 88. Yeast strain YBT25 expressing MSH2p with the amino acid substitutions C195R (SEQ ID NO: 211), G350R (SEQ ID NO: 587), H658R (SEQ ID NO: 79), G702R (SEQ ID NO: 216), C716R (SEQ ID NO: 82), and G770R (SEQ ID NO: 83) exhibited mutation frequencies of 280 to 410×10⁻⁵ (Table 7). These mutation frequencies correspond to mutation defects of 70 to 103. Statistical analyses of the data from each independent experiment (not shown) indicated that the mutation frequencies conferred by the C195R, G350R, H658R, G702R, C716R, and G770R substitutions were statistically greater than the level exhibited by strain YBT25 expressing wild-type yeast MSH2p and were not significantly different from strain YBT25 containing pMETc. Therefore, these results demonstrate that amino acid substitutions C195R, G350, H658R, G702R, C716R, and G770R in MSH2p result in complete loss-of-MMR function.

Strain YBT25 expressing MSH2p with amino acid substitutions P30L (SEQ ID NO. 583), T44M (SEQ ID NO: 532), Q61P (SEQ ID NO: 207), N123S (SEQ ID NO: 208), D163H (SEQ ID NO: 209), N182S (SEQ ID NO: 75), C195W (SEQ ID NO. 585), G317V (SEQ ID NO: 586), S318C (SEQ ID NO: 533), C345Y (SEQ ID NO: 77), P361L (SEQ ID NO. 588), L402F (SEQ ID NO: 213), L402V (SEQ ID NO: 214), L521P (SEQ ID NO: 215), E580V (SEQ ID NO. 592), N601S (SEQ ID NO. 593), A627V (SEQ ID NO: 78), G702V (SEQ ID NO: 217), M707I (SEQ ID NO: 218), I710T (SEQ ID NO: 80), V741I (SEQ ID NO. 597), I754V (SEQ ID NO: 219), I789V (SEQ ID NO: 84), and K873E (SEQ ID NO: 220) exhibited mutation frequencies of 0.6 to 8.0×10⁻⁵ as measured using the (GT)₁₆G::URA3 allele (Table 7). Statistical analyses of the data from each independent experiment (not shown) indicated that these mutation frequencies were not significantly different from the mutation frequency exhibited by YBT25 expressing wild-type yeast MSH2p. Therefore, these results demonstrate that the P30L, T44M, Q61P, N123S, D163H, N182S, C195W, G317V, S318C, C345Y, P361L, L402F, L402V, L521P, E580V, N601S, A627V, G702V, M707I, I710T, V741I, I754V, I789V and K873E amino acid substitutions do not detectably alter M function as measured by GT-tract instability.

Ten of the codon alterations in MSH2 gave rise to proteins which exhibited intermediate levels of MMR activity. Strain YBT25 expressing MSH2p with amino acid substitutions VE106/107-del (SEQ ID NO. 584), E194G (SEQ ID NO: 210), A267V (SEQ ID NO: 212), C345R (SEQ ID NO: 76), P456-del (SEQ ID NO. 589), L457P (SEQ ID NO: 590), R552C (SEQ ID NO. 591), L613R (SEQ ID NO. 594), D621N (SEQ ID NO. 595), P640T (SEQ ID NO. 596), and G711R (SEQ ID NO: 81) exhibited mutation frequencies of 4.7 to 280×10⁻⁵ as measured using the (GT)₁₆G::URA3 allele (Table 7). Statistical analyses of the data from each independent experiment (not shown) indicated that the mutation frequencies were significantly different from that exhibited by YBT25 containing either pMETc/MSH2 or pMETc. Therefore, the results indicate that the VE106/107-del, E194G, A267V, C345R, P456-del, L457P, R552c, L613R, D621N, P640T, and G711R amino acid substitutions confer a reduced, but not complete, loss-of-MMR function i.e. partial function in MMR.

To confirm the functional results obtained using the (GT)₁₆G::URA3 allele a second MMR assay based on forward mutation to canavanine resistance was carried out. This assay detects mainly base substitutions and frameshift mutations in mononucleotide tracts in the arginine permease (CAN1) gene (49). The results show that for the majority of alterations (n=34 of 41) the functional results obtained using the CAN1 allele were similar to those obtained using (GT)₁₆G::URA3 (Table 7). Interestingly, the L521P substitution, which gave no increase in the mutation frequency as measured by the (GT)₁₆G::URA3 allele, conferred a considerable increase in the mutation frequency as measured by the canavanine resistance assay. It is possible that the L521P substitution causes aberrant recognition and/or processing of mutations in mononucleotide tracts, which occur in the CAN1 gene, while allowing normal processing of mutations in dinucleotide repeats, which occur in the (GT)₁₆G::URA3 allele. A structural basis for this assertion exists because amino acid residue 521 lies immediately adjacent to a region of the protein known to be important for recognition of mismatched DNA (50-52). Additional experiments are needed to explore DNA mismatch recognition and/or processing by MSH2p containing the L521P alteration. The E194G, A267V, C345R P456-del, L613R, D621N, and P640T alterations, which conferred partial loss of MMR activity using the (GT)₁₆G::URA3 allele, did not confer notable increases in the mutation frequency using canavanine resistance as an end point. These alterations may have minimal effects on the repair of common canavanine resistance mutations. Alternatively, it is possible that the sensitivity of the canavanine resistance assay was too low to detect the rather slight defects in MMR function conferred by these amino acid alterations.

In summary, codon alterations which lead to the amino acid substitutions C195R, G350R, H658R, G702R, C716R, and G770R are considered inactivating mutations. Alterations leading to amino acid substitutions P30L, T44M, Q61P, N123S, D163H, N182S, C195W, G317V, S318C, C345Y, P361L, L402F, L402V, E580V, N601S, A627V, G702V, M707I, I710T, V741I, I754V, I789V and K873E are classified as silent polymorphisms. Alterations VE106/107-del, E194G, A267V, C345R, P456-del, L457P, R552c, L613R, D621N, P640T, and G711R are classified as efficiency polymorphisms because they confer intermediate levels of MMR activity using the most sensitive reporter gene [(GT)₁₆G::URA3]. The substitution L521P is also classified as an efficiency polymorphism because it appears to partially impair MMR activity, albeit in an DNA mismatch-specific manner. The corresponding amino acid alterations in the human MSH2 protein (see Tables 6 and 6a) are considered to have an equivalent effect on R activity.

### Example 5

### Construction and Functional Analysis of Hybrid Human-Yeast MSH2 Genes

Rationale. Approximately 44% of the MSH2 nucleotide alterations observed in the human population are predicted to alter an amino acid residue which is not conserved in the yeast MSH2p. To address this issue, the construction and functional characterization of hybrid human-yeast hybrid genes that contain regions of human MSH2p replacing the homologous region of yeast MSH2p are reported herein. Except for the noted chimeric region, the structure of each hybrid genes is identical to the parental expression vector pMETc/MSH2, which contains the native yeast MSH2 gene expressed from the MET25 promoter (see Example 4).

Plasmids. Hybrid human-yeast MSH2 genes encoding chimeric MSH2 proteins were constructed using pMETc/MSH2 as the parental vector (see Example 4). MSH2_h(1-63). This hybrid human-yeast gene was constructed using a two-piece overlap extension PCR method. A 230-bp 5′-end fragment of human MSH2 was amplified by PCR from ATCC cDNA clone #7520190 (American Type Culture Collection, Rockville, Md.) using primers SEQ ID NO: 221 and SEQ ID NO: 222. A 1.8-kb fragment from the central portion of yeast MSH2 was amplified from plasmid pMETc/MSH2 using primers SEQ ID NO: 223 and SEQ ID NO: 224. PCR amplifications were carried out using Pfu Turbo DNA polymerase (Stratagene, La Jolla Calif.) using the manufacturer's recommended conditions. The two fragments were mixed in approximately equimolar amounts and subjected to overlap extension PCR using primers SEQ ID NO: 221 and SEQ ID NO: 224. The overlap extension PCR product was digested with BamHI and NcoI and ligated into BamHI-NcoI digested pMETc/MSH2, replacing the equivalent portion of the yeast gene. The plasmid containing MSH2_h(1-63) was verified by restriction fragment length polymorphism (RFLP) analysis. The protein (SEQ ID NO: 103) encoded by this gene contains amino acids 1-63 of human MSH2p and 64-964 of yeast MSH2p. MSH2_h(621-832). Methods for the construction of this gene have been described in an earlier patent application (WO 02/081624 A3, published Oct. 17, 2002). The protein (SEQ ID NO: 537) encoded by this gene contains amino acids 1-638 and 861-964 of yeast MSH2p and amino acids 621-832 of human MSH2p. MSH2_h(621-739). An approximately 650-bp 3′-end fragment of yeast MSH2 was amplified from S. cerevisiae strain S288C genomic DNA using the primers SEQ ID NO: 225 and SEQ ID NO: 226. The fragment was digested with Bsu36I and XhoI and ligated into Bsu36I-XhoI digested pMETc/MSH2_h(621-832), replacing the equivalent portion of the hybrid human-yeast sequence. The protein (SEQ ID NO: 104) encoded by this gene contains amino acids 1-638 and 759-964 of yeast MSH2p and amino acids 621-739 of human MSH2p. MSH2_h(730-832). An approximately 1.5-kb fragment of yeast MSH2 was amplified from S. cerevisiae strain S288C genomic DNA using the primers SEQ ID NO: 227 and SEQ ID NO: 228. The fragment was digested with SphI and Bsu36I and ligated into SphI-Bsu36I digested pMETc/MSH2_h(621-832), replacing the equivalent portion of the hybrid human-yeast sequence. The protein (SEQ ID NO: 534) encoded by this gene contains amino acids 1-748 and 861-964 of yeast MSH2p and amino acids 730-832 of human MSH2p. MSH2_h(621-832)ins9. This hybrid human-yeast gene was constructed using a two-piece overlap extension PCR method. A 700-bp 5′-end fragment of yeast MSH2 was amplified by PCR from pMETc/MSH2_h(621-832) using primers SEQ ID NO: 229 and SEQ ID NO: 226. A 450-bp fragment from the central portion of hybrid MSH2_h(621-832) was also amplified from plasmid pMETc/MSH2_h(621-832) using primers SEQ ID NO: 230 and SEQ ID NO: 231. Note that primers SEQ ID NO: 229 and SEQ ID NO: 231 contain at their 5′ ends 24 and 27 bases, which are complimentary to each other and encode yeast MSH2p amino acids 827-835 (“ins9”). PCR amplifications were carried out using Pfu Turbo DNA polymerase (Stratagene, La Jolla Calif.) using the manufacturer's recommended conditions. The two fragments were mixed in approximately equimolar amounts and subjected to overlap extension PCR using primers SEQ ID NO: 230 and SEQ ID NO: 226. The overlap extension PCR product was digested with Bsu36I and XhoI and ligated into Bsu36I-XhoI digested pMETc/MSH2_h(621-832), replacing the equivalent portion of the hybrid yeast gene. The plasmid containing MSH2_h(621-832) was verified by restriction fragment length polymorphism (RFLP) analysis using an Eco47III site added by primer SEQ ID 229. The protein (SEQ ID NO: 535) encoded by this gene is identical to that encoded by MSH2_h(621-832) except for the insertion of yeast residues 827-835, between human residues 807-808. MSH2_h(730-832)ins9. Methods for construction of this hybrid gene were similar to those used for MSH2_h(621-832)ins9, except that plasmid pMETc/MSH2-h(730-832) was used for cloning and amplification of PCR fragments. The protein (SEQ ID NO: 536) encoded by this gene is identical to that encoded by MSH2_h(730-832) except for the insertion of yeast residues 827-835 (“ins9”), between human residues 807-808.

Site directed mutations. Mutations were introduced into the hybrid human-yeast MSH2_h(621-739) gene using the QuikChange Site-Directed Mutagenesis kit (Stratagene, La Jolla, Calif.) following the manufacturer's instructions. Plasmid pMETc/MSH2_h(621-739) was used as template for the following variants (yeast alterations given): A636P (SEQ ID NO: 85), E647K (SEQ ID NO: 86), Y656H (SEQ ID NO: 87), and M729V (SEQ ID NO: 88). Sense and antisense oligonucleotide primers were obtained from BioSynthesis Inc. (Lewisville, Tex.) and, to facilitate screening for mutant clones, included a silent restriction site change in addition to the desired missense alteration (Table 6).

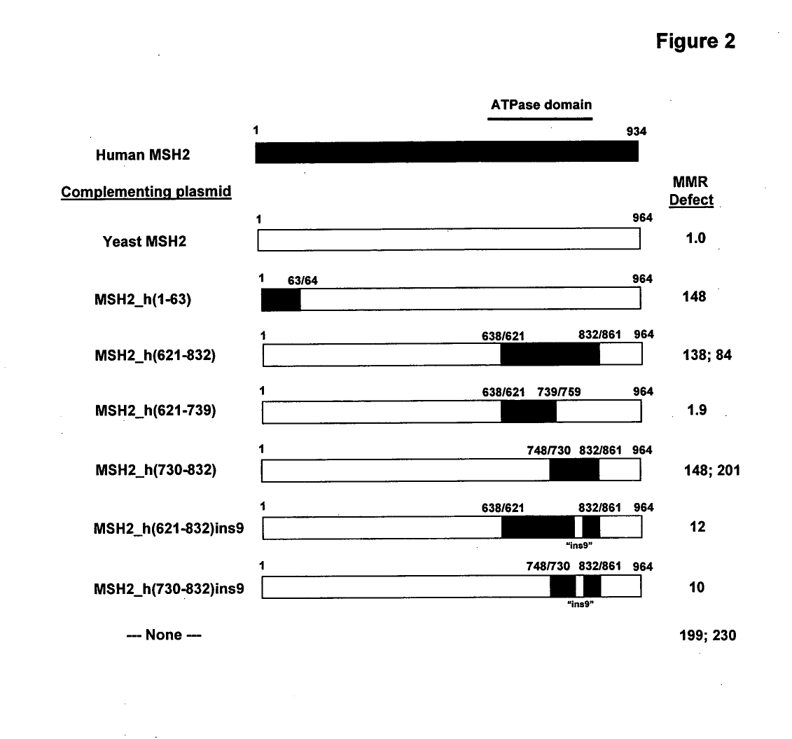

Results. Six hybrid human-yeast MSH2 genes were constructed by replacing a region of the yeast MSH2p coding sequence with the homologous region of the human MSH2p (FIG. 2). Plasmids carrying the hybrid human-yeast MSH2 genes were introduced into yeast strain YBT25 containing pSH91 and standardized MMR assays were carried out as described previously (see Example 1). Strain YBT25 containing pMETc, which lacks an MSH2 gene, exhibited mutation frequencies of 328×10⁻⁵ while the same strain containing pMETc/MSH2 exhibited mutation frequencies of 1.6×10⁻⁵ (Table 8, Experiment #1). These results represent mutation defect of 199 for cells lacking functional MSH2p. The mutation frequencies of YBT25 expressing MSH2 proteins MSH2_h(1-63), MSH2_h(621-832), MSH2_h(621-739), and MSH2_h(730-832) were 245×10⁻⁵, 228×10⁻⁵, 3.2×10⁻⁵, and 245×10⁻⁵, respectively (Table 8, Experiment #1). The hybrid MSH2_h(1-63) did not appear to confer notable levels of MMR activity it can be concluded that this region of the human MSH2p can not functionally substitute for the homologous yeast MSH2 region. Similarly, hybrid MSH2_h(621-832), which contains a 212 amino acid portion of the human MSH2p ATPase domain, also was non-functional in MMR. However, when this portion of the ATPase domain was split into two smaller portions, one of the subsequent hybrids [MSH2_h(621-739)] conferred notable levels of MMR activity while the other [MSH2_h(730-832)] was non-functional. The active human yeast hybrid protein MSH2_h(621-739) contains a 119-amino acid portion of human MSH2p and exhibited a mutation defect of 2 compared to wild-type yeast MSH2p.

To achieve MMR activity in non-functional hybrids, hybrids MSH2_h(621-832) and MSH2_h(730-832) were modified to encode yeast amino acids 827-KNLKEQKHD-835 (“ins9”) between human residues 807-808 of these hybrids (FIG. 2). This 9-amino acid portion of yeast MSH2p is absent from the equivalent portion of the human region and thus may be an important feature for function of the protein in yeast. Consistent with this postulate, hybrids MSH2_h(621-832)ins9 and MSH2_h(730-832)ins9 exhibited substantial function in MMR compared to the parental hybrids which did not contain the “ins9” peptide (Table 8, Experiment #2). Amino acids 827-835 appear to be critical for function of MSH2p in yeast and may play a role in binding and/or hydrolysis of ATP. Alternatively, these residues may provide a surface necessary for important protein:protein interactions in yeast. The availability of functional hybrid human yeast MSH2 genes increases the number of human codon alterations which can be functionally evaluated in yeast. The aforementioned experiments show that all human variants within codons 621-832 (≈23% of the full length protein) can now be functionally tested in yeast.

To demonstrate the utility of the human-yeast hybrid MSH2 proteins four human missense codons, which occur at amino acid residues that are not conserved in the yeast protein, were tested for their effects on MMR activity. Site-directed mutations were made in plasmid pMETc/MSH2_h(621-739) to generate missense codons identical to those previously observed in the human population (Table 6). The variant MSH2 genes and control plasmids pMETc/MSH2_h(621-739) and pMETc were transformed into YBT25 containing pSH91 and tested for activity in the standardized MMR assay (Example 1). As measured using the (GT)₁₆G::URA3 allele, strain YBT25 containing the pMETc expression vector, which lacks an MSH2 gene, exhibited a mean mutation frequency of 258×10⁻⁵ (Table 8, Experiment #3). The same strain expressing MSH2-h(621-739) exhibited a mean mutation frequency of 8.5×10⁻⁵ (Table 8, Experiment #3). The A636P, E647K, Y656H, and M729V variants conferred mutation frequencies of 239×10⁻⁵, 8.9×10⁻⁵, 5.5×10⁻⁵, and 12×10⁻⁵, respectively. The results indicate that A636P is an inactivating mutation and E647K, Y656H, and M729V are silent polymorphisms.

### Example 6

### Construction of an ADE2 Gene Containing a Microsatellite and Generation of a Yeast Strain Exhibiting Single-Colony Colorimetric Indicators of DNA Mismatch Repair Function

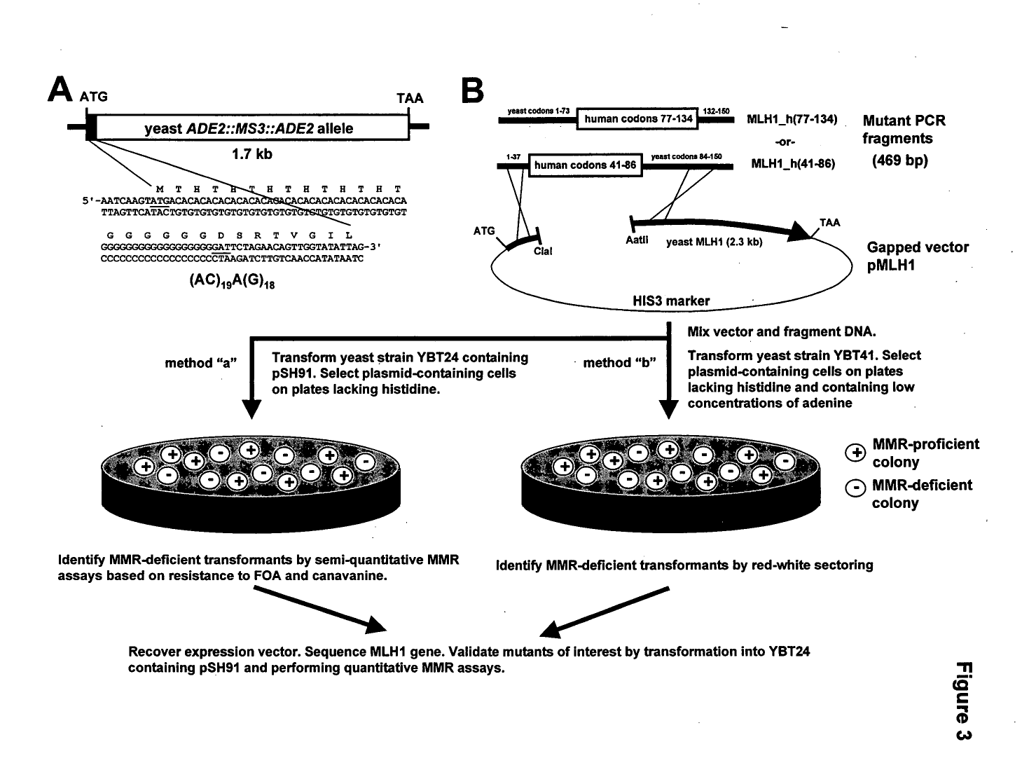

A microsatellite sequence was introduced at the 5′ end of the yeast ADE2 gene coding sequence (SEQ ID NO: 618) as follows. The yeast ADE2 translation initiation codon and 187 bp 5′ flanking DNA coding sequence was PCR amplified from S. cerevisiae S288C DNA using the primers SEQ ID NO: 232 and SEQ ID NO: 233. The ADE2 coding sequence from codon 2 to 36 bp 3′ to the termination codon were PCR amplified from S. cerevisiae S288C DNA using the primers SEQ ID NO: 234 and SEQ ID NO: 235. The approximately 216 bp and 1808 bp DNA fragments were mixed in approximately equimolar amounts and subjected to overlap extension PCR amplification (53) using primers SEQ ID NO: 232 and SEQ ID NO: 235. The predominant PCR product was the approximately 1998 bp overlap extension product. Accurate overlap extension in this reaction would yield an ADE2 gene with the DNA sequence SEQ ID NO: 236 at the 5′ end, inserted between the first (ATG) and second (GAT) codons of the ADE2 gene. This modified gene is termed ADE2::MS3::ADE2 (SEQ ID NO: 619). When translated in yeast, this gene would encode a fusion protein with the amino acids SEQ ID NO: 237 inserted between the first and second amino acid residues of the native yeast ADE2p (FIG. 3A).

The DNA from the overlap extension PCR amplification was purified using the Wizard DNA Purification kit (Promega, Madison, Wis.) and introduced by transformation (36) into either S. cerevisiae strain YBT24; pSH91 (Example 1) or YBT25; pSH91 (Example 4). Transformants were selected on plates lacking adenine (SD, H, Ly). Individual transformants were subsequently grown in liquid cultures in SD, H, Ly, diluted and plated for single colonies on plates containing low concentrations of adenine (SD, H, Ly, 4 μg/mL adenine). As described previously (54), cells that do not express the ADE2 gene form pink colonies on, these plates due to the accumulation of an intermediate in adenine biosynthesis. The individual transformants grown in liquid culture (above) were screened for those that formed a high percentage of sectored colonies on low adenine plates. These represent strains with an unstable ADE2 gene (mutates at a high frequency), presumably because the native chromosomal gene was replaced by the overlap extension product containing a microsatellite in the ADE2 coding sequence. One clone from each transformation was shown to have the native ADE2 chromosomal gene replaced by the microsatellite-containing gene by PCR amplification of chromosomal DNA using ADE2-specific and microsatellite-specific primers (data not shown). Strain YBT39 has the genotype MATα ADE2::MS3::ADE2 his3-Δ200 leu2-Δ1 lys2-801 trp1-Δ63 ura3-52 msh2Δ::LEU2 and strain YBT40 has the genotype MATα ADE2::MS3::ADE2 his 3-Δ200 leu2-Δ1 lys2-801 trp1-Δ63 ura3-52 mlh1Δ::LEU2, where MS3 refers to SEQ ID NO: 236 inserted between the first and second codons of the native ADE2 gene coding sequence.

Similar procedures were used to construct another yeast strain containing an mlh1 chromosomal gene disruption and the microsatellite containing ADE2 gene, except that a larger ADE2 targeting sequence was used at the 5′ end. The yeast ADE2 translation initiation codon and 644 bp 5′ flanking DNA coding sequence was PCR amplified from S. cerevisiae S288C DNA using the primers SEQ ID NO: 238 and SEQ ID NO: 233. The ADE2 coding sequence from codon 2 to 36 bp 3′ to the termination codon was PCR amplified from S. cerevisiae S288C DNA using the primers SEQ ID NO: 234 and SEQ ID NO: 235. The approximately 673 bp and 1808 bp DNA fragments were mixed in approximately equimolar amounts and subjected to overlap extension PCR amplification (53) using primers SEQ ID NO: 238 and SEQ ID NO: 235. The predominant PCR product was the approximately 2452 bp overlap extension product. The overlap extension PCR product was purified and used to transform strain YBT24; pSH91 selecting for adenine prototrophs. Individual transformants were screened as above to identify clones with an unstable ADE2 gene. Yeast strain YBT41 was shown to have the native ADE2 chromosomal gene replaced by the microsatellite-containing gene by PCR amplification of chromosomal DNA using ADE2-specific and microsatellite-specific primers (data not shown). Strain YBT41 has the genotype MATα ADE2::MS3::ADE2 his3-Δ200 leu2-Δ1 lys2-801 trp1-Δ63 ura3-52 mlh1Δ::LEU2, where MS3 refers to SEQ ID NO: 236 inserted between the first and second codons of the native ADE2 gene coding sequence.

The above strains were transformed with either the empty expression vector pMETc or pMETc containing an appropriate yeast mismatch gene for that particular strain. The transformed yeast strains were grown in liquid cultures lacking adenine, diluted and plated on plates containing 4 μg/mL adenine (100-250 colonies per plate). After two days growth at 30° C. and two days growth at room temperature, the plates were evaluated for colony color with the results summarized in Table 9. A number of plates from each strain were examined and the range of colony colors observed under these conditions is indicated. The results demonstrate that with wild-type DNA mismatch repair function (chromosomal gene disruption complemented by plasmid expressed wild type gene), all the cells on plates containing low adenine form normal white colonies. In the absence of a plasmid expressed gene, however, a significant percentage of the cells form pink and/or sectored colonies due to mutation of the ADE2-MS3-ADE2 gene. Such sectored colonies are not observed when the mismatch repair deficient strains contain a native ADE2 gene (data not shown) indicating that the high mutation rate is due to the introduced microsatellite sequence (MS3). Strain YBT41 consistently had a higher frequency of sectored colonies (for reasons that are not clear at this time).

### Example 7

### Additional Functional Analysis of Hybrids MLH1_h(41-86) and MLH1_h(77-134)

Plasmids. Plasmids pMLH1_h(41-86) and pMLH1_h(77-134) are identical to pMLH1 (see Example 3) but contain codons encoding human MLH1p amino acid residues 41-86 and 77-134, respectively, in place of the homologous codons of yeast MLH1 (26).

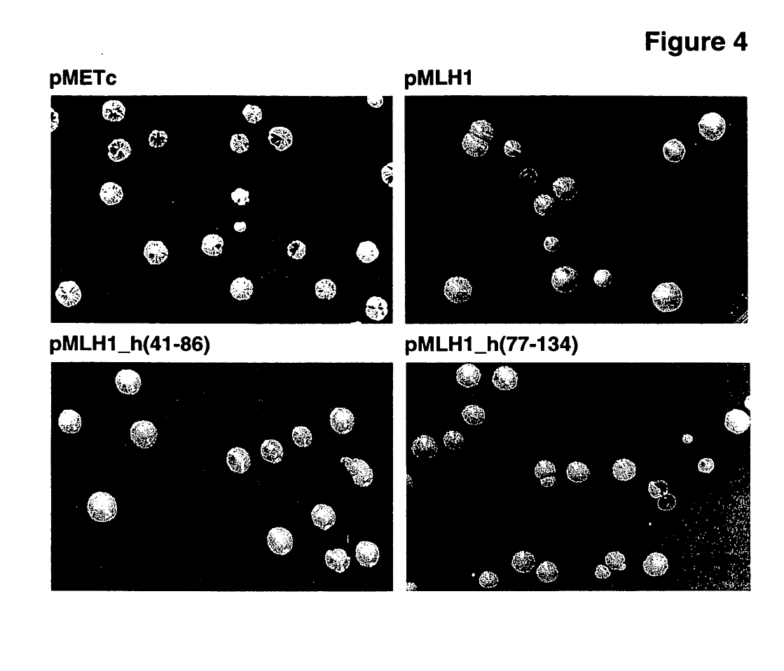

Results. The human-yeast hybrid genes MLH1_h(41-86) (SEQ ID NO: 118) and MLH1_h(77-134) (SEQ ID NO: 119) encode chimeric MLH1 proteins that contain 46 and 58 amino acid regions, respectively, of human MLH1p replacing the homologous region of yeast MLH1p. When expressed in haploid yeast cells containing a deletion of the chromosomal MLH1 gene these hybrids were active in MMR in a standardized in vivo assay that measures the frequency of frameshift mutations in an in-frame (GT)₁₆G microsatellite preceding the URA3 gene (26, 34). In the present invention, the function of MLH1_h(41-86) and MLH1_h(77-134) was confirmed and extended using in vivo MMR assays that employ other reporter genes. The first assay involved transformation of the haploid-yeast strain YBT41, which contains an MLH1 deletion and the ADE2::MS3::ADE2 allele (FIG. 3A), and this assay allowed a determination of MMR proficiency based on the color of individual colonies (Example 6). Strain YBT41 was transformed with pMLH1, pMLH1_h(41-86), pMLH1_h(77-134) and pMETc, the expression vector lacking an MLH1 gene, and histidine prototrophs were selected on plates containing low concentrations (4 μg/ml) of adenine (FIG. 4). When YBT41 was transformed with pMETc, and thus did not express MLH1p, >95% of the colonies were red-white sectored (Table 10, “None”). This sectoring is probably due to instability of the in-frame MS3 microsatellite resulting in frameshift mutations in the ADE2 gene. In contrast, when transformed with pMLH1, a vector expressing the wild-type yeast MLH1 gene, <2% of the colonies were sectored. It is likely that the integrity of the in-frame ADE2::MS3::ADE2 allele is maintained by the MMR process such that >98% of the colonies appeared white. When YBT41 was transformed with hybrids MLH1_h(41-86) and MLH1_h(77-134) the percentage of colonies exhibiting a red-white sectored appearance was 14% and <2%, respectively (Table 10).

In the second MMR assay yeast colonies were grown in liquid culture and assayed for forward mutation to canavanine resistance as described in Example 4. Yeast strain YBT24 (mlh1Δ) was transformed with pMLH1, pMLH1_h(41-86), pMLH1_h(77-134) and pMETc and individual colonies were assayed by fluctuation tests to determine CAN1 mutation rates (Table 10). YBT24 containing the empty expression vector pMETc exhibited a mutation frequency of 3.1×10⁻⁵ while the strain expressing the native yeast MLH1 gene (pMLH1) exhibited a mutation frequency of 7.1×10⁻⁷. This represents a mutation defect of 44 for yeast cells lacking MLH1p. The mutation frequencies of yeast cells expressing MLH1_h(41-86) and pMLH1_h(77-134) were 2.8×10⁻⁶ and 1.7×10⁻⁶, respectively, which corresponded to mutation defects of 4.0 and 2.4. Taken together the results demonstrate that MLH1 proteins encoded by MLH1_h(41-86) and MLH1_h(77-134) are functional in the repair of a variety of DNA mismatch structures. Although the mutation frequencies exhibited by cells expressing the human-yeast hybrid genes are slightly elevated compared to those levels exhibited by cells expressing the native yeast MLH1 gene, the mutation frequencies conferred by the hybrids are at least 10-fold lower than those levels exhibited by yeast cells lacking any functional MLH1p. The complementation efficiencies for MLH1_h(41-86) and MLH1_h(77-134) are consistent with previous studies (26), and show that MLH1_h(77-134) may be slightly more proficient than MLH1_h(41-86) in MMR.

### Example 8

### Identification of Novel MLH1 Variants that Cause Loss of Mismatch Repair Function

Technology for the selection, screening and identification of MLH1 mutations causing loss-of-MMR was described in a previous patent application (WO 02/081624 A3, published Oct. 17, 2002). As described therein use of this technology led to the isolation of 39 MLH1p variants which contain a single amino acid alteration which confers loss-of-MMR activity (27). In this invention, the original method and a new, novel method (Method “b”, described below) were used to isolate additional MLH1p variants which lack MMR function and thus, may be used for the diagnosis of cancer susceptibility.

Error-prone PCR and in vivo gap repair cloning. Pools of mutant MLH1 gene fragments were generated by error-prone PCR using Mutazyme™ (a component of the GeneMorph PCR mutagenesis kit; Stratagene, La Jolla, Calif.) or Taq (Promega, Madison, Wis.) DNA polymerases, which have different misincorporation biases (55). The use of both enzymes should ensure that pools of mutagenized DNA are representative of all possible base substitutions. XhoI-linearized plasmids pMLH1_h(41-86) and pMLH1_h(77-134) were used as templates in PCR mixes containing the buffers, nucleotides, and enzyme concentrations recommended by the manufacturer of each DNA polymerase. The upstream and downstream primers were SEQ ID NO: 35 and SEQ ID NO: 239, respectively, which amplify a 401-bp fragment spanning the human portion of each hybrid MLH1 gene. In preliminary experiments the upstream primer SEQ ID NO: 240 was used to generate a fragment of 475-bp. The protocol for temperature cycling was: 94° C./2 min; 33 cycles of 94° C./36 sec, 55° C./1 min, 72° C./2 min; and 72° C./10 min. Conditions of high and low fidelity were manipulated by varying the amount of template DNA (3-74 ng) in reactions containing Mutazyme and the MgCl₂ concentration (1.5-2.5 mM) in reactions containing Taq DNA polymerase. PCR fragments were purified with Wizard™ PCR preps (Promega, Madison, Wis.) and used for in vivo gap repair cloning in yeast (54, 56, 57). Briefly, 0.5 μg purified PCR product was combined with 0.4 μg ClaI-AatII digested pMLH1 vector and the DNA mixture was co-transformed into YBT24 or YBT41 containing pSH91. Yeast cells in which fragment and vector recombine were converted to histidine prototrophy due to the presence of the HIS3 marker gene on the pMLH1 expression vector. This process typically yielded ≈500 transformants (i.e. colonies) per plate; while equivalent transformations performed with restricted vector alone exhibited very few (<5) colonies per plate.

Semi-quantitative assays for screening of MMR activity. Screening of transformants for MMR proficiency was carried out using either of two methods depending on whether YBT24 or YBT41 was the host strain for in vivo gap repair cloning. Method “a”: When gap repair cloning was carried out in YBT24 containing pSH91, transformants were assayed sequentially using a spot test for FOA resistance and a patch test for canavanine (CAN) resistance exactly as described previously (27). Briefly, individual clones from the transformation were grown in 3 ml SD (0.67% yeast nitrogen base without amino acids, 2% dextrose) medium containing adenine and lysine (Day 1 culture) and the next day 120 μl of the saturated culture was subinoculated into 3 ml fresh SD medium containing adenine, lysine and uracil. The addition of uracil in the medium allows growth of cells containing a ura3 mutation arising from a frameshift in the (GT)₁₆G-tract of pSH91. These ura3 mutants exhibit a 5-fluoroorotic acid (FOA)-resistant phenotype (25, 34). Following 24 hours growth, 4 μl of the culture was spotted in duplicate on SD plates containing adenine, lysine, uracil and 1 mg/ml FOA (Toronto Research Chemicals Inc., ON, Canada). The plates were incubated at 30° C. for 48 hours and then scored by counting the number of FOA-resistant colonies on each spot. Transformants that exhibited few colonies (<15; typically 0 to 5) per spot were scored as having low levels of MSI (i.e. MMR proficient) and were not analyzed further. Transformants that exhibited many colonies (≧15; typically 20 to 50) per spot were scored as having high levels of MSI (i.e. MMR deficient) and were arrayed on a master plate by applying 25 μl the Day 1 cultures to SD plates containing adenine and lysine. These clones were subjected to a secondary assay based on spontaneous forward mutations in the arginine permease gene (CAN1), which cause resistance to canavanine. A 1 μl loopful of cells from the arrayed transformants were patched out on SD plates containing adenine, lysine and 60 μg/ml canavanine. Plates were incubated three days at 30° C. and scored by counting the number of canavanine-resistant colonies. Yeast clones that exhibited few colonies (<15) were scored as having low levels of genetic instability (i.e. normal in MMR) and were not analyzed further. Clones that exhibited many colonies (≧15; typically 30 to 100) were selected for further analysis. Method “b”: When in vivo gap repair was carried out in yeast strain YBT41 (see Examples 6 and 7), the transformed cells were plated directly on SD plates containing low concentrations (4 μg/ml) of adenine and incubated for 4-5 days. As described previously (54), cells that do not express the ADE2 gene form red colonies due to the accumulation of an intermediate in adenine biosynthesis while cells expressing a wild-type ADE2 gene form white colonies. When the ADE2::MS3::ADE2 allele is unstable (i.e. mutates to ade- at a high frequency due to instability of the MS3 microsatellite) the strain forms a white colony with red sectors on plates containing low adenine (see Example 6). In Method “b”, after gap repair transformation and plating on low adenine, colonies that exhibit abundant red-white sectoring were selected for further analysis. This method allowed single-step cloning and identification of MMR-deficient transformants since MMR deficient cells exhibit red-white sectoring directly on transformation plates (containing low concentrations of adenine).

Preparation of Yeast DNA and Isolation of Mutant MLH1 Expression Vectors. Total Yeast DNA was prepared from 15 ml liquid cultures using the glass-bead method (58) and resuspended in 50 μl H₂O. To recover mutant plasmids from the yeast strain a 15 μl aliquot of each DNA sample was digested with BamHI, which restricts the pSH91 expression vector but not the MLH1 expression vector, and shuttled into E. coli strain DH5α by electroporation using a BTX ECM399 system (Genetronics, Inc., San Diego, Calif.). Bacterial colonies were selected by growth on LB plates containing 50 μg/ml ampicillin and plasmid DNA was purified using the Wizard Plus SV Minipreps kit (Promega, Madison, Wis.).

DNA sequencing. DNA sequencing was performed at commercial facilities using dye-terminator chemistry and automated sequencers (ABI models 377 and 3700, Applied Biosystems, Foster City, Calif.). Chromatogram and text files were analyzed with Chromas (version 1.45, http://technelysium.com.au/chromas.html) and GeneRunner (version 3.04, Hastings Software Inc.) software, respectively. Sequencing was carried out in both the forward and reverse directions using primers SEQ ID NO: 241, SEQ ID NO: 239, SEQ ID NO: 242 and/or SEQ ID NO: 243.

Quantitative in vivo MMR assays. Standardized MMR assays based on mutation to ura3 FOA^{R} were performed as described previously (see Example 1).

MLH1p accession numbers, alignment and mutation databases. Homo sapiens, NP_{—}000240; Mus musculus, Q9JK91; Rattus norvegicus, NP-112315; Drosophila melanogaster, NP_{—}477022; Saccharomyces cerevisiae, NP_{—}013890; Schizosaccharomyces pombe, NP_{—}596199; Arabidopsis thaliana, NP_{—}567345; Caenorhabditis elegans, NP_{—}499796; Escherichia coli, NP_{—}418591; Staphylococcus aureus, Q93T05. Sequences were retrieved from the Protein Database of the National Center for Biotechnology Information (http://www.ncbi.nlm.nih.gov) and aligned using ClustalW (http://www.ebi.ac.uk/clustalw/). Human MLH1 alterations referenced in the text were reported in one or more of the following public mutation databases: International Collaborative Group on HNPCC (http://www.nfdhtl.nl), Human Gene Mutation Database (http://www.uwcm.ac.uk) and Swiss-protein (http://www.expasy.ch). The databases were last examined Aug. 29, 2003.

Results. To generate mutations in MLH1, 5′-end fragments of the MLH1_h(41-86) and MLH1_h(77-134) genes were synthesized by error-prone PCR and cloned directly in yeast by gap-repair transformation (FIG. 3B). Three hundred to 1000 colonies, each representing a cloned PCR fragment, were obtained for each gap-repair transformation. Two methods based on semi-quantitative MMR assays were employed to identify colonies having a deficiency in MMR. In initial experiments strain YBT24 containing pSH91 was used for in vivo gap repair (Method “a”). Colonies were screened for resistance to FOA (ura3) by a spot test and those that exhibited a high number of FOA-resistant colonies compared to transformants containing unmutagenized hybrid plasmid were then subjected to canavanine patch analysis to confirm the MMR-deficient phenotype. The second screening method (Method “b”) utilized strain YBT41 (Example 6) for in vivo gap repair and was based on the selection of red-white sectored (i.e., MMR-deficient) colonies.

Hybrid human-yeast MLH1 expression plasmids were isolated from 387 transformants that exhibited MMR deficiency. DNA sequencing revealed that 60 of the transformants harbored hybrid MLH1 genes that were identical to the unmutagenized parental gene. This number of false-positives was not surprising considering the observation that yeast carrying an functional MLH1 gene occasionally exhibit a mutator phenotype (Table 10 and data not shown). The origin of these false-positives remains undetermined but it is possible that the mutator phenotype in these clones results from a spontaneous mutation in another endogenous MMR gene or the presence of pre-existing mutations in the reporter gene before transformation with an MLH1 gene. The remaining 327 sequenced genes exhibited at least one alteration in the mutagenized region. More specifically, there were 24 (7.3%) hybrid MLH1 genes that contained a frameshift mutation and 16 (4.9%) that contained a termination codon (Table 11). The identification of these types of mutations validated the screening strategy because they would be expected to encode truncated MLH1 proteins that lack MMR function. There were 129 (39%) plasmids that contained multiple (2 or more) alterations in the hybrid MLH1 genes and these were not analyzed further. Finally, there were 158 (48%) hybrid MLH1 genes that contained a single missense codon; these represented the most abundant type of alteration found in the screen. To verify that these missense mutations were bona fide loss-of-MMR function mutations, the isolated plasmids containing each variant gene was re-introduced into the parental strain (YBT24) for quantitative MMR assays (based on stability of the (GT)₁₆G microsatellite in pSH91; see below).

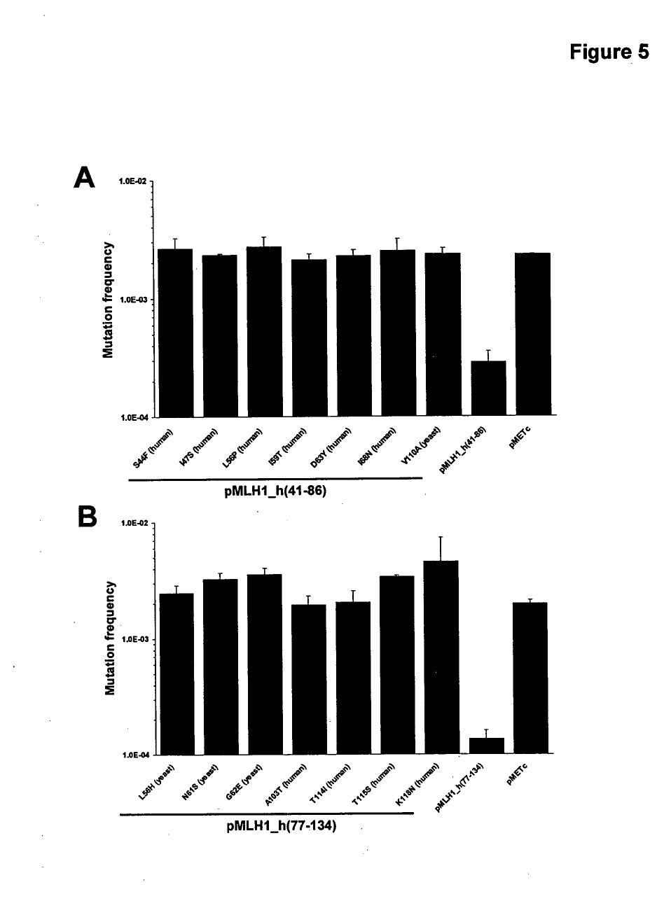

Mutation frequencies of YBT24; pSH91 containing mutant hybrid MLH1 genes were determined and compared to those levels exhibited by YBT24; pSH91 containing the appropriate parental hybrid gene or the empty expression vector pMETc and results for representative variants are depicted in FIG. 5. Mutation frequencies of 2.1-2.7×10⁻³ were exhibited by yeast cells carrying hybrid MLH1_h(41-86) genes with S44F, 147S, L56P, 159T, D63Y, 168N substitutions in the human portion of the hybrid and a V110A substitution in the yeast portion. These mutation frequencies were similar to the mutation frequency conferred by the pMETc expression vector (2.3×10⁻³) and approximately 10-fold greater than the mutation frequency conferred by the parental hybrid MLH1_h(41-86) (2.9×10⁻⁴) indicating that these variants confer a significant loss-of-MMR function. Hybrid MLH1_h(77-134) genes with A103T, T114I, T115S and K118N substitutions in the human portion and L56H, N61S and G62E substitutions in the yeast portion conferred mutation frequencies of 2.0-4.6×10⁻³. These mutation frequencies were similar to the mutation frequency conferred by the pMETc expression vector (2.0×10⁻³) and were approximately 20-fold greater than the mutation frequency conferred by the parental hybrid MLH1_h(77-134) (1.4×10⁻⁴), indicating that these variants also confer a significant loss-of-MMR function. All of the hybrid MLH1 genes containing single missense codons were verified by quantitative in vivo MMR assays as described above and the results are listed in Tables 12 and 13.

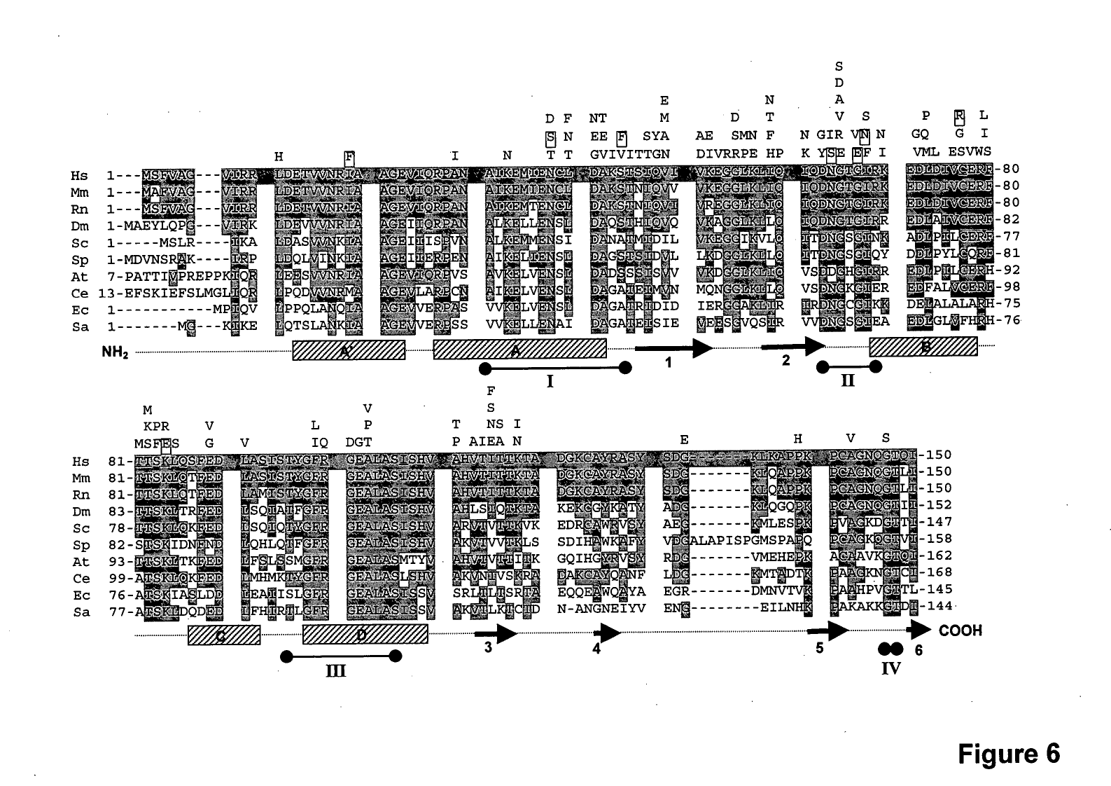

Each of the 158 MLH1 variants containing a missense codon was tested in quantitative MMR assays as described above. To confirm loss-of-MMR function, we assigned a level of 2 or greater for the mutation defect. This level represents a mutation frequency twice as high as the parental MLH1_h(41-86) and MLH1_h(77-134) hybrids and exceeds the maximal levels typically observed for these hybrids (Tables 12 and 13, footnotes). The results of the quantitative MMR assays demonstrated that 151 of the isolated variants (representing 124 non-redundant alterations) exhibited a mutation defect of 2.1 or more. As listed in Tables 12 and 13 [for variants of MLH1_h(41-86) and MLH1_h(77-134), respectively] a range of MMR defects was apparent and the vast majority of missense codons conferred a substantial loss-in-MMR function (Tables 12 and 13, ++ and +++). In addition to amino acid substitutions that impaired MMR activity, seven amino acid substitutions conferred little-to-no loss of MMR in qualitative assays and were classified as silent polymorphisms (Table 14). Variants containing these alterations probably arose as false-positives in the prospective screen. A comparison of the amino acid substitutions which had a deleterious effect on MMR activity revealed four alterations [D41G (human)/D38G (yeast), T45I (human)/T42I (yeast), E53V (human)/E50V (yeast), 168N (human)/165N (yeast)] that predict identical amino acid substitutions in the equivalent human and yeast residues. Additionally, three alterations [137T (yeast), F80L (human), G144S (yeast)] were isolated in the same codon using different hybrids. Identification of equivalent mutations in different hybrids further supports the notion that these substitutions confer detrimental effects on MMR function. In total, 117 unique amino acid substitutions in the NH₂-terminal end of MLH1p have been shown to cause a loss-of-MMR function. As compared to an alignment of MLH1p orthologs, the majority of these substitutions occur at highly conserved amino acid residues (FIG. 6). Interestingly, eight substitutions (corresponding to human MLH1p I19F, N38S, S44F, N64S, G67E, I68N, C77R and K84E) have been previously reported as possible pathogenic mutations in the human population.

This particular example illustrates a novel method for the identification of new human MMR gene sequences which result in MMR proteins that do not function in MMR and hence, if carried by and individual, cause a predisposition to develop cancer. The method employs the yeast Saccharomyces cerevisiae, which has been used previously for the identification mutant MMR genes. For example, Jeyaprakash et al. (1996) used genetic complementation experiments and then direct cloning and DNA sequencing to ascertain the identity of the mutant gene in yeast strains with preexisting defects in microsatellite stability. More recent reports describe global mutagenesis of yeast DNA, selection of yeast strains for those having alterations in MMR gene activity followed by cloning and DNA sequencing (59-62). It should be noted that these studies were focused on finding variants of the native yeast (not human) proteins and exploring the structure and function of the mutant in yeast. Indeed, if reported at all, expression of the human MMR proteins in yeast has either no known effect (ex. MSH2, MSH3, MSH6) or causes a dominant negative phenotype i.e. the normal human protein causes a significant increase the yeast's mutation rate (MLH1, and the MSH2-MSH6 heterodimer) (63, 64). Previous studies have attempted to bypass these impediments by using, for example, an hMSH2-ADE2 fusion gene to screen for stop codons in the hMSH2 coding sequence or assays based on gain or loss of the dominant mutator phenotype (63-65). However, these assays do not reflect the biological effect of the protein. We have solved this problem by inventing hybrid human-yeast MMR proteins (see WO 02/081624 A3, published Oct. 17, 2002; and Examples 2 and 5 herein) that retain their biological function for MMR. These hybrids have allowed the development of biologically relevant assays in yeast for identification of human MMR gene mutations. To date, the most similar work relating to this aspect of the present invention was published WO 02/081624 A3. However the method described here is based on a colorimetric screen using a novel yeast strain and ADE2 reporter gene and has the important advantages of being more rapid and reliable than the method described earlier. It should also be pointed out that ADE2 reporter genes have been used in two of the aforementioned reports (61, 65). Although ADE2-based reporter genes are commonly used in yeast due to their utility in colorimetric cell-based screens, the present ADE2 reporter is presumed to be novel (ADE2::MS3::ADE2) and contains a microsatellite sequence, which we have developed and engineered into the 5′ end of the gene. These reagents and refinement of their performance characteristics have resulted in a method which, it is believed, could not have been predicted based on earlier work and should have important clinical utility for determinations of an individual's susceptibility to develop cancer.

### Example 9

### Functional Analysis of MLH1p Having Missense Alterations at Human Codon S44

Rationale. A spectrum of codon alterations at human MLH1 codon 44 were analyzed to provide functional information about MLH1 amino acid substitutions and to investigate how genetic variability at a single codon affects MMR activity. As reported in a previous patent application (WO 02/081624 A3, published Oct. 17, 2002), 13 of the 20 possible amino acid substitutions at MLH1 residue S44 were assayed for their effects on MMR. In this invention functional information on the remaining 6 amino acid substitutions (S44M, S44N, S44K, S44D, S44E, and S44G) has been determined.

Plasmids. Oligonucleotides SEQ ID NO: 105 (for S44M), SEQ ID NO: 106 (for S44N), SEQ ID NO:107 (for S44K), SEQ ID NO: 108 (for S44D), SEQ ID NO:109 (for S44E), and SEQ ID NO:110 (for S44G) were obtained from Bio-Synthesis Inc. (Lewisville, Tex.). Each oligonucleotide was used in combination with oligonucleotide SEQ ID NO: 111 to amplify a 122-bp portion of the human MLH1 gene from cDNA clone ATCC#217884 (American Type Culture Collection, Rockville, Md.) Amplification was carried out by PCR and utilized Pfu DNA polymerase (Stratagene, La Jolla, Calif.) according to the manufacturer's instructions. The PCR cycling conditions were as follows: 95° C. for 2 min; 33 cycles of 95° C. for 36 sec, 55° C. for 1 min, 72° C. for 2 min; and 72° C. for 10 min. The resulting fragments were digested with ClaI and AatII, which cleave at sites introduced in the PCR primers, and ligated into ClaI-AatII digested pMLH1 replacing a portion of the native yeast MLH1 gene. This cloning strategy generates yeast expression vectors identical to that encoding the human-yeast hybrid MLH1_h(41-86) (SEQ ID NO: 118) except for the indicated amino acid replacement. Plasmids encoding the hybrid MLH1 proteins MLH1_h(41-86)S44M (SEQ ID NO: 112), MLH1_h(41-86)S44N (SEQ ID NO: 113), MLH1_h(41-86)S44K (SEQ ID NO: 114), MLH1_h(41-86)S44D (SEQ ID NO: 115), MLH1_h(41-86)S44E (SEQ ID NO: 116), and MLH1_h(41-86)S44G (SEQ ID NO: 117), were introduced into the mlh1-deletion strain YBT24 containing pSH91 and functionally tested in the standardized MMR assay (see Example 1). Three independent mutant clones for each variant were tested with identical results. One clone was sequenced in both directions to confirm the appropriate codon change and validate the PCR-amplified sequence of the hybrid molecule. The mutation frequencies below a derived from replicate cultures of a single mutant clone that had been confirmed by DNA sequencing.

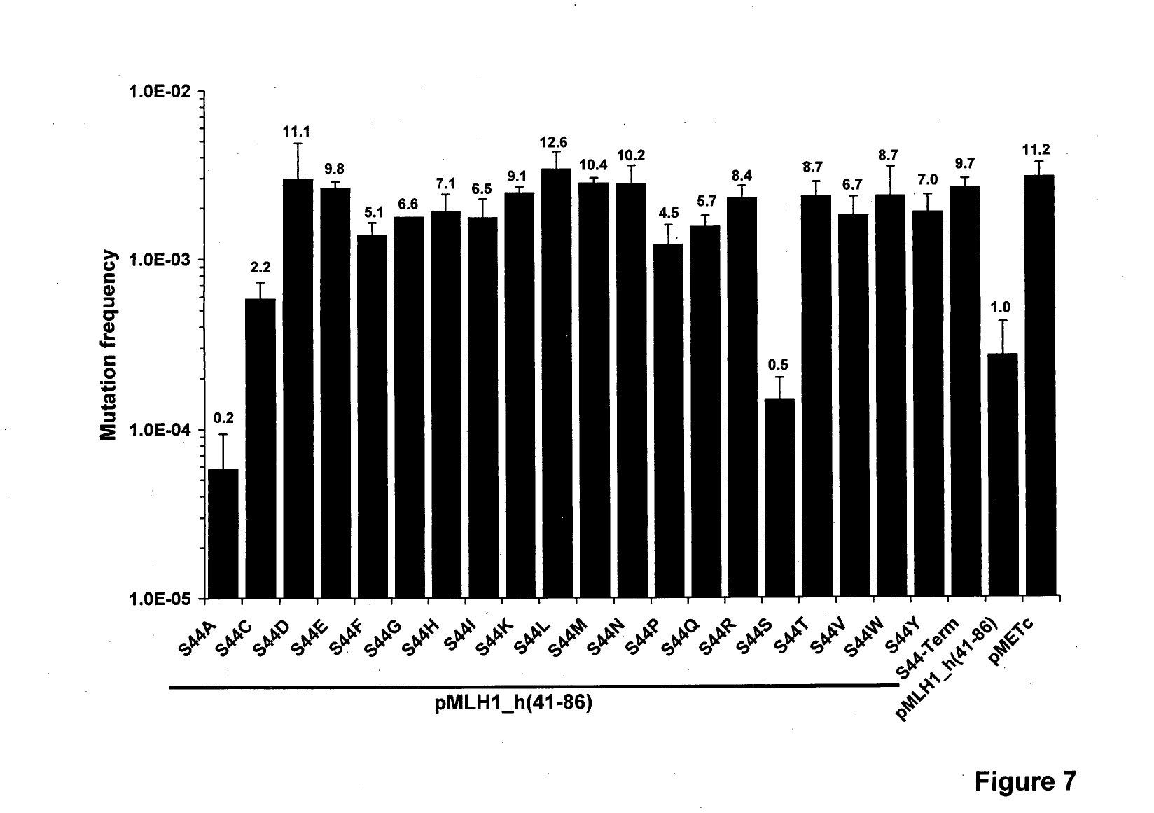

Results. As shown in FIG. 7, the mutation frequency YBT24 containing pSH91 and the hybrid MLH1_h(41-86) was 2.67×10⁻⁴. In contrast, the strain YBT24 containing pSH91 and the expression vector pMETc (lacking an MLH1 gene) was 3.0×10⁻³. The elevated mutation frequency exhibited by the mlh1-deficient strain represents a mutation defect of 11.2. Expression vectors containing the hybrid MLH1_h(41-86) with the substitutions S44D, S44E, S44G, S44K, S44M, and S44N exhibited mutation frequencies from 1.76 to 2.96×10⁻³ (FIG. 7). These values represent mutation defects of 11.1, 9.8, 6.6, 9.1, 10.4, and 10.2 respectively. The results indicate that MLH1 proteins containing the alterations S44M, S44N, S44K, S44D, S44E, and S44G exhibit impaired MMR activity.

The functional information reported here combined with data from a previous patent application (WO 02/081624 A3, published Oct. 17, 2002) completes the analysis of human MLH1 codon 44. In summary, 18 of 20 amino acids at codon 44 result in substantial loss-of-MMR function (FIG. 7). Only codons which encode serine (S) (the wild-type human amino acid) and alanine (A) (the wild-type yeast amino acid at the corresponding position) gave rise to a protein with levels of MMR activity. Interestingly, an alignment of the amino acid sequences of MLH1 from 8 other species ranging from E. coli to mouse shows that only serine and alanine appear in this position (Example 6). Because the majority of amino acid substitutions at residue 44 lead to loss-of-MMR function, it is concluded that genetic variability at this codon must be quite limited in order to maintain proper function of the MLH1 protein.

### Example 10

### Functional Analysis of Mlh1p Having Missense Alterations at Human Codon K43

Plasmids. An oligonucleotide with the sequence 5′-CTG TAT CGA TGC ANN NTC CAC AAG TAT TCA AGT G-′3 (SEQ ID NO: 120), where “N” represents any of the four nucleotides A, C, G, or T, was obtained from Bio-Synthesis Inc. (Lewisville, Tex.). The random incorporation of nucleotides at this triplet, creates the possibility for a collection of oligonucleotides containing all 64 possible codon (encoding all 20 possible amino acids) alterations at this position. Oligonucleotide SEQ ID NO: 120 in combination with oligonucleotide SEQ ID NO: 111, was then used to amplify a 122-bp portion of the hMLH1 gene using hMLH1 cDNA clone ATCC#217884 as a template. Amplification utilized Pfu DNA polymerase (Stratagene, La Jolla, Calif.) according to the manufacturer's instructions and cycling conditions were as follows: 95° C. for 2 min; 33 cycles of 95° C. for 36 sec, 55° C. for 1 min, 72° C. for 2 min; and 72° C. for 10 min. The resulting fragment was digested with ClaI and AatII and ligated into pMLH1 replacing the corresponding portion of the native MLH1 gene. Cloning generated a pool of molecules identical to pMLH1_h(41-86) except for the randomized codon at hMLH1 codon 43. Transformation into E. coli DH5a generated a collection of colonies that each contain a genetically different pMLH1_h(41-86) molecule. Plasmid DNA from individual colonies was purified using Wizard Plus SV Minipreps (Promega, Madison, Wis.) and then analyzed by DNA sequencing to confirm the sequence of the amplified region and, importantly, to determine the codon present at hMLH1 position 43. Plasmids containing codons for 13 of the 20 possible amino acid substitutions were identified in this way. Plasmids containing codons for the 7 remaining amino acid substitutions were generated by direct cloning of PCR products. Briefly, oligonucleotides SEQ ID NO: 244 (for K43C), SEQ ID NO: 245 (for K43E), SEQ ID NO: 246 (for K43H), SEQ ID NO: 247 (for K43K), SEQ ID NO: 248 (for K43P), SEQ ID NO: 249 (for K43Q) and SEQ ID NO: 250 (for K43W) were obtained from Bio-Synthesis Inc. (Lewisville, Tex.). Each oligonucleotide was used in combination with oligonucleotide SEQ ID NO: 111 to amplify a 122-bp portion of the human MLH1 gene from cDNA clone ATCC#217884 (American Type Culture Collection, Rockville, Md.) Amplification was carried out by PCR and utilized Pfu DNA polymerase (Stratagene) according to the manufacturer's instructions. The PCR cycling conditions were as follows: 95° C. for 2 min; 33 cycles of 95° C. for 36 sec, 55° C. for 1 min, 72° C. for 2 min; and 72° C. for 10 min. The resulting fragments were digested with ClaI and AatII, which cleave at sites introduced in the PCR primers, and ligated into ClaI-AatII digested pMLH1 replacing a portion of the native yeast MLH1 gene. The plasmids were verified by DNA sequencing. MLH1_h(41-86) expression plasmids containing all possible amino acid substitutions were transformed into YB24 containing pSH91. Mutation frequencies were determined using the standardized quantitative MMR assay as described in Example 1. The mean mutation frequency ±standard deviation of two to nine independent cultures is shown.

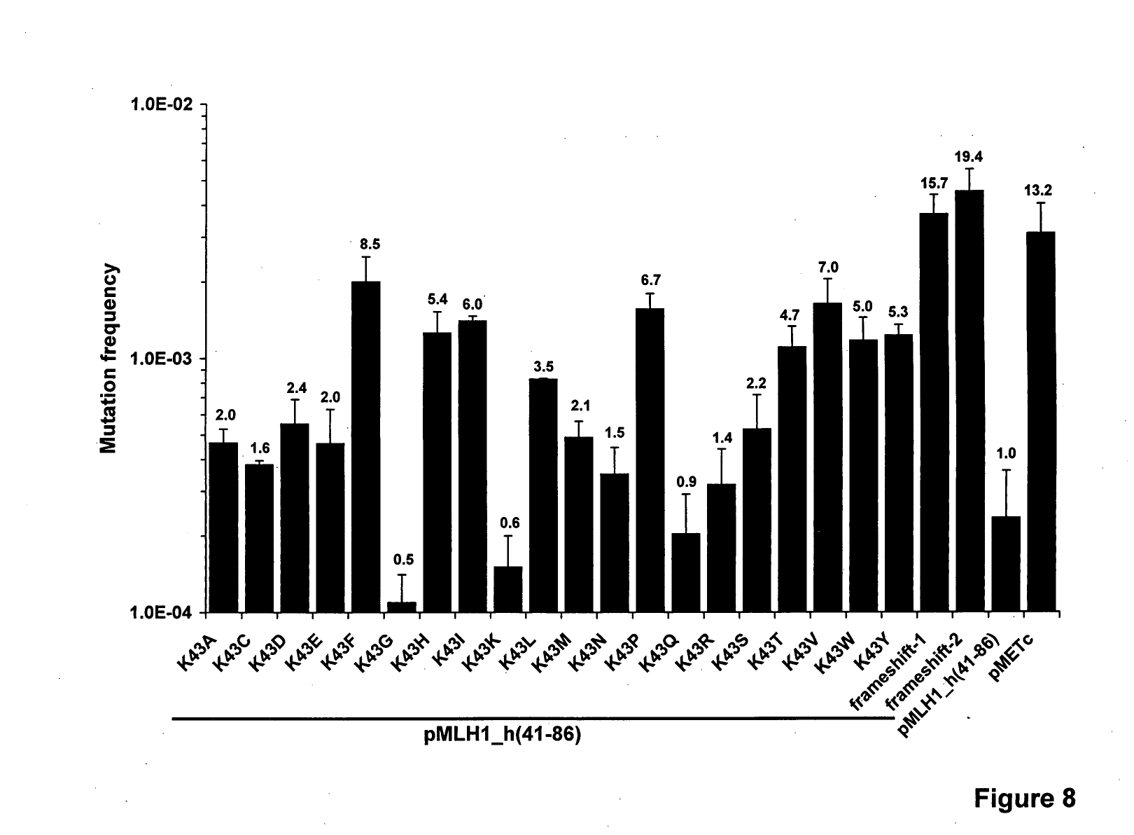

Results. As shown in FIG. 8, the mutation frequency YBT24 containing pSH91 and the hybrid MLH1_h(41-86) was 2.34×10⁻⁴. In contrast, the strain YBT24 containing pSH91 and the expression vector pMETc (lacking an MLH1 gene) was 3.10×10⁻³. The elevated mutation frequency exhibited by the mlh1-deficient strain represents a mutation defect of 13.2. As expected, MLH1_h(41-86) containing a silent K43K alteration (SEQ ID NO: 254) exhibited MMR activity comparable to the parental hybrid MLH1_h(41-86), while proteins with spontaneous deletions in codons 43 (“frameshift-1”) and 45 (“frameshift-2”) exhibited mutation frequencies that were not significantly different from that conferred by the empty expression vector pMETc (FIG. 8).

Of the 19 possible MLH1_h(41-86) variants having a amino acid substation at codon 43, fourteen [K43A (SEQ ID NO: 123), K43D (SEQ ID NO: 121), K43E (SEQ ID NO: 252), K43F (SEQ ID NO: 145), K43H (SEQ ID NO: 253), K43I (SEQ ID NO: 127), K43L (SEQ ID NO: 128), K43M (SEQ ID NO: 124), K43P (SEQ ID NO: 255), K43S (SEQ ID NO: 126), K43T (SEQ ID NO: 147), K43V (SEQ ID NO: 146), K43W (SEQ ID NO: 257) and K43Y (SEQ ID NO: 122)] conferred mutation frequencies between 4.6×10⁻⁴ and 2.0×10⁻³ (FIG. 8). These values represent mutation defects of 2.0 to 8.5. The remaining 5 alterations [K43C (SEQ ID NO: 251), K43G (SEQ-ID NO: 143), K43N (SEQ ID NO: 144), K43Q (SEQ ID NO: 256) and K43R (SEQ ID NO: 125)] conferred mutation frequencies of 1.1×10⁻⁴ to 3.8×10⁻⁴ values that represent a mutation defect of 1.6 or less and thus have little or no effect on protein function. While substitutions at residue S44 tended to be more severe (conferring a mutation defect of 5.0 or greater) than those at residue K43, the vast majority of substitutions at both codons impaired MMR activity to some degree. Interestingly, for both residues K43 and S44, the range of substitutions that resulted in little to no loss-of-MMR function closely mirrored the variability observed in nature (FIG. 6).

### ABBREVIATIONS

CRC: colorectal cancer

HNPCC: hereditary nonpolyposis colorectal cancer

MMR: DNA mismatch repair

PCR: polymerase chain reaction

NY: a codon at position N in a gene (N denoting the number of the codon, where the ATG translation initiation codon is assigned number 1) which encodes the amino acid X (encoding one of the twenty amino acids, the symbols for which are listed below).

XNY: a codon at position N in a gene (N denoting the number of the codon, where the ATG translation initiation codon is assigned number 1) in which the codon for amino acid X (encoding one of the twenty amino acids, the symbols for which is below) has been changed to codon Y (again represented by one of the twenty symbols below).

A: the amino acid alanine

C: the amino acid cysteine

D: the amino acid aspartic acid

E: the amino acid glutamic acid

F: the amino acid phenylalanine

G: the amino acid glycine

H: the amino acid histidine

I: the amino acid isoleucine

K: the amino acid lysine

L: the amino acid leucine

M: the amino acid methionine

N: the amino acid asparagine

P: the amino acid proline

Q: the amino acid glutamine

R: the amino acid arginine

S: the amino acid serine

T: the amino acid threonine

V: the amino acid valine

W: the amino acid tryptophan

Y: the amino acid tyrosine

### REFERENCES

1. Kinzler, K. W. and Vogelstein, B. (1996) Lessons from hereditary colorectal cancer. Cell, 87(2), 159-170.
2. Papadopoulos, N. and Lindblom, A. (1997) Molecular Basis of HNPCC: Mutations of MMR Genes. Human Mutation, 10, 89-99.
3. Peltomaki, P. and de la Chapelle, A. (1997) Mutations predisposing to hereditary nonpolyposis colorectal cancer. Adv. Cancer Res., 71, 93-119.
4. Lynch, H. T. and de la Chappelle, A. (1999) Genetic susceptibility to non-polyposis colorectal cancer. J. Med. Genet., 36, 801-818.
5. Peltomaki, P. (2001) Deficient DNA mismatch repair: a common etiologic factor for colon cancer. Hum. Mol. Genet., 10(7), 735-740.
6. Mitchell, R. J., Farrington, S. M., Dunlop, M. G. and Campbell, H. (2002) Mismatch repair genes hMLH1 and hMSH2 and colorectal cancer: a HuGE review. Am. J. Epidemiol., 156(10), 885-902.
7. Vasen, H. F., Mecklin, J. P., Khan, P. M. and Lynch, H. T. (1991) The International Collaborative Group on Hereditary Non-Polyposis Colorectal Cancer (ICG-HNPCC). Dis. Colon Rectum, 34(5), 424-425.
8. Fishel, R. and Wilson, T. (1997) MutS homologs in mammalian cells. Curr. Opin. Genet. Dev., 7, 105-113.
9. Jiricny, J. and Nystrom-Lahti, M. (2000) Mismatch repair defects in cancer. Curr. Opin. Genet. Dev., 10, 157-161.
10. Kolodner, R. D. and Marsischky, G. T. (1999) Eukaryotic DNA mismatch repair. Curr. Opin. Genet. Dev., 9, 89-96.
11. Herman, J. G., Umar, A., Polyak, K., Graff, J. R., Ahuja, N., Issa, J. P., Markowitz, S., Willson, J. K. V., Hamilton, S. R., Kinzler, K. W., Kane, M. F., Kolodner, R. D., Vogelstein, B., Kunkel, T. and Baylin, S. B. (1998) Incidence and functional consequences of hMLH1 promoter hypermethylation in colorectal carcinoma. Proc. Natl. Acad. Sci. USA, 95, 6870-6875.
12. Kolodner, R. D. (2000) Guarding against mutations. Nature, 407, 687-689.
13. Cahill, D. P., Kinzler, K. W., Vogelstien, B. and Lengauer, C. (1999) Genetic Instability and Darwinian Selection in Tumours. Trends in Biochemical Sciences, 24(12), M57-M60.
14. Hanahan, D. and Weinberg, R. A. (2000) The hallmarks of cancer. Cell, 100(1), 57-70.
15. Loeb, L. A. (1991) Mutator phenotype may be required for multistage carcinogenesis. Cancer Res., 51, 3075-3079.
16. Tomlinson, I. P., Novelli, M. R. and Bodner, W. F. (1996) The mutation rate and cancer. Proc. Natl. Acad. Sci. USA, 93(14800-14803).
17. Lengauer, C., Kinzler, K. W. and Vogelstein, B. (1998) Genetic Instabilities in human cancers. Nature, 396, 643-649.
18. Loeb, L. A. (2001) A mutator phenotype in cancer. Cancer. Res., 61, 3230-3239.
19. Ron, E. (1998) Ionizing radiation and cancer risk: evidence from epidemiology. Radiat. Res., 150(5 Suppl), S30-41.
20. Cleaver, J. and Crowley, E. (2002) UV damage, DNA repair and skin carcinogenesis. Fron. Biosci., 7, d1024-1043.
21. Hecht, S. S. (2003) Tobacco carcinogens, their biomarkers and tobacco-induced cancer. Nat. Rev. Cancer, 3(10), 733-744.
22. Cleaver, J. E. and Kraemer, K. H. (1995) Xeroderma pigmentosum and Cockayne syndrome, 7th Ed. The metabolic and molecular basis of inherited disease (Scriver, C., Beaudet, A., Sly, W., and Valle, D., Eds.), McGraw-Hill, New York.
23. Ban, C. and Yang, W. (1998) Crystal structure and ATPase activity of MutL: implications for DNA repair and mutagenesis. Cell, 95, 541-552.
24. Tran, P. T. and Liskay, M. R. (2000) Functional studies on the candidate ATPase domains of Saccharomyces cerevisiae MutL. Mol. Cell. Biol., 20, 6390-6398.
25. Polaczek, P., Putzke, A. P., Leong, K. and Bitter, G. A. (1998) Functional genetic tests of DNA mismatch repair protein activity in Saccharomyces cerevisiae. Gene, 213, 159-167.
26. Ellison, A. R., Lofing, J. and Bitter, G. A. (2001) Functional analysis of human MLH1 and MSH2 missense variants and hybrid human-yeast MLH1 proteins in Saccharomyces cerevisiae. Hum. Mol. Genet., 10(18), 1889-1900.
27. Bitter, G. A. and Ellison, A. R. (2002). BTOL Corp., USA.
28. Beck, N. E., Tomlinson, I. P., Homfray, T., Hodgson, S. V., Harcopos, C. J. and Bodmer, W. F. (1997) Genetic testing is important in families with a history suggestive of hereditary non-polyposis colorectal cancer even if the Amsterdam criteria are not fulfilled. Br. J. Surg., 84(2), 233-237.
29. Syngal, S., Fox, E. A., Li, C., Dovidio, M., Eng, C., Kolodner, R. D. and Garber, J. E. (1999) Interpretation of genetic test results for hereditary nonpolyposis colorectal cancer: implications for clinical predisposition testing. JAMA, 282(3), 247-253.
30. Terdiman, J. P., Gum Jr., J. R., Conrad, P. G., Miller, G. A., Weinberg, V., Crawley, S. C., Levin, T. R., Reeves, C., Schmitt, A., Hepburn, M., Sleisenger, M. H. and Kim, Y. S. (2001) Efficient detection of hereditary nonpolyposis colorectal cancer gene carriers by screening for tumor microsatellite instability before germline testing. Gastroenterology, 120, 21-30.
31. Giraldo, A., Gomez, A., Salguero, A., Garcia, H., Aristizabal, F., Gutierrez, O., Angel, L. A., Padron, J., Martinez, C., Martinez, H., Malayer, O., Florez, L. and Barvo, R. (2003) hMLH1 and hMSH2 mutations in Colombian families with HNPCC (abstract). Am J. Hum. Genet., 73 (suppl.)(5), 230.
32. Woods, M. O., Green, J. S., Robb, D., Pollett, A., Younghusband, B., Gallinger, S., Parfrey, P. S., McLaughlin, J. R. and Bapat, B. (2003), American Association for Cancer Research, 94th Annual Meeting. Cadmus Professional Communications, Toronto, Ontario, Canada, Vol. 44, pp. 1367.
33. Mumberg, D., Muller, R. and Funk, M. (1994) Regulatable promoters of Saccharomyces cerevisiae: comparison of transcriptional activity and their use for heterologous expression. Mol. Gen. Genet., 22(25), 5767-5768.
34. Strand, M., Prolla, T. A., Liskay, R. M. and Petes, T. D. (1993) Destabilization of tracts of simple repetitive DNA in yeast by mutations affecting DNA mismatch repair. Nature, 365, 274-276.
35. Sikorski, R. S. and Hieter, P. (1989) A system of shuttle vectors and yeast host strains designed for efficient manipulation of DNA in Saccharomyces cerevisiae. Genetics, 122(1), 19-27.
36. Ito, H., Fukuda, Y., Murata, K. and Kimura, A. (1983) Transformation of intact yeast cells treated with alkali cations. J. Bacteriol., 153, 163-168.
37. Godfrey, K. (1985) Statistics in practice: comparing the means of several groups. N Engl J Med, 313(23), 1450-1455.
38. Ryder, E. F. and Robakiewicz, P. (1998) In Ausubel, F. M., Brent, R., Kingston, R. E., Moore, D. D., Seidman, J. G., Smith, J. A., and Struhl, K. (eds.), Current protocols in molecular biology. John Wiley and Sons, New York, Vol. supple 43, pp. A.3I.1-22.
39. Nakahara, M., Yokozaki, H., Yasui, W., Dohi, K. and Tahara, E. (1997) Identification of concurrent germ-line mutations in hMSH2 and/or hMLH1 in Japanese hereditary nonpolyposis colorectal cancer kindreds. Cancer Epidemiol. Biomarkers Prevent., 6, 1057-1064.
40. Guerrette, S., Wilson, T., Gradia, S. and Fishel, R. (1998) Interactions of human hMSH2 with, hMSH3 and hMSH2 with hMSH6: examination of mutations found in hereditary nonpolyposis colorectal cancer. Mol. Cell. Biol., 18(11), 6616-6623.
41. Holinski-Feder, E., Muller-Koch, Y., Friedl, W., Moeslein, G., Keller, G., Plaschke, J., Ballhausen, W., Gross, M., Baldwin-Jedele, K., Jungck, M., Mangold, E., Vogelsang, H., Schackert, H. K., Lohsea, P., Murken, J. and Meitinger, T. (2001) DHPLC mutation analysis of the hereditary nonpolyposis colon cancer (HNPCC) genes hMLH1 and hMSH2. J. Biochem. Biophys. Methods, 47(1-2), 21-32.
42. Samowitz, W. S., Curtin, K., Lin, H. H., Robertson, M. A., Schaffer, D., Nichols, M., Gruenthal, K., Leppert, M. F. and Slattery, M. L. (2001) The colon cancer burden of genetically defined hereditary nonpolyposis colon cancer. Gastroenterology, 121, 830-838.
43. Otway, R., Tetlow, N., Homby, J. and Kohonen-Corish, M. (2000) Evaluation of enzymatic mutation detection in hereditary nonpolyposis colorectal cancer. Human Mutation, 16(1), 61-67.
44. Gorlov, I. P., Gorlov, O. Y., Frazier, M. L. and Amos, C. I. (2003) Missense mutations in hMLH1 and hMSH2 are associated with exonic splicing enhancers. Am. J. Hum. Genet., 73, 1157-1161.
45. Jeong, S.-Y., Shin, K.-H., Shin, J.-H., Ku, J.-L., Shin, Y.-K., Park, S.-Y., Kim, W.-H. and Park, J.-G. (2003) Microsatellite instability and mutations in DNA mismatch repair genes in sporatic colorectal cancers. Dis. Colon Rectum, 46, 1069-1077.
46. Nafa, K., Peterlongo, P., Shia, J., Canale, L., Lerman, G., Glogowski, E., Guillem, J., Markowitz, A., Offit, K. and Ellis, N. A. (2003) Mutational analysis of the mismatch repair genes in HNPCC patients. Am. J. Hum. Genet., 73 (suppl.)(5), 238.
47. Wagner, A., Barrows, A., Wijnen, J. T., van der Klift, H., Franken, P. F., Verkuijlen, P., Nakagawa, H., Geugien, M., Jaghmohan-Changur, S., Breukel, C., Meijers-Heijboer, H., Morreau, H., van Puijenbroek, M., Burn, J., Coronel, S., Kinarski, Y., Okimoto, R., Watson, P., Lynch, J. F., de la Chapelle, A., Lynch, H. T. and Fodde, R. (2003) Molecular analysis of hereditary nonpolyposis colorectal cancer in the United States; high mutation detection rate among clinically selected families and characterization of an American founder mutation. Am. J. Hum. Genet., 72, 1088-1100.
48. Lea, D. E. and Coulson, C. A. (1949) The distribution of the numbers of mutants in bacterial populations. J. Genet., 49, 264-285.
49. Marsischky, G. T., Filosi, N., F., K. M. and Kolodner, R. (1996) Redundancy of Saccharomyces cerevisiae MSH3 and MSH6 in MSH2-dependent mismatch repair. Genes Dev., 10, 407-420.
50. Lamers, M. H., Perrakis, A., Enzlin, J. H., Winterwerp, H. H. K., de Wind, N. and Sixma, T. K. (2000) The Crystal Structure of DNA Mismatch Repair Protein MutS binding to a G:T Mismatch. Nature, 407, 711-717.
51. Obmolova, G., Ban, C., Hsieh, P. and Yang, W. (2000) Crystal Structure of Mismatch Repair Protein MutS and its Complex with a Substrate DNA. Nature, 407, 703-710.
52. Drotschmann, K., Yang, W., Brownewell, F. E., Kool, E. T. and Kunkel, T. A. (2001) Asymmetric recognition of DNA local distortion. J. Biol. Chem., 276(49), 46225-46229.
53. Bitter, G. A. (1998) Function of hybrid human-yeast cyclin-dependent kinases in Saccharomyces cerevisiae. Mol. Gen. Genet., 260, 120-130.
54. Bitter, G. A., Schaeffer, T. N. and Ellison, A. E. (2002) Reporter gene regulation in Saccharomyces cerevisiae by the human p53 tumor suppressor protein. J. Mol. Micro. Biotechnol., 4(6), 539-550.
55. Cline, J. and Hogrefe, H. (2000) GeneMorph™ PCR mutagenesis kit produces a unique mutational spectrum. Stratagies Newsletter (Stratagene), 13(4), 157-161.
56. Scharer, E. and Iggo, R. (1992) Mammalian p53 can function as a transcription factor in yeast. Nucleic Acids Res., 20, 1539-1545.
57. Ishioka, C., Frebourg, T., Yan, Y. X., Vidal, M., Friend, S. H., Schmidt, S. and Iggo, R. (1993) Screening patients for heterozygous p53 mutations using a functional assay in yeast. Nat. Genet., 5(2), 124-129.
58. Hoffman, C. S. and Winston, F. (1987) A ten-minute DNA preparation from yeast efficiently releases autonomous plasmids for transformation of Escherichia coli. Gene, 57, 267-272.
59. Studamire, B., Price, G., Sugawara, N., Haber, J. and Alani, E. (1999) Separation-of-function mutations in Saccharomyces cerevisiae MSH2 that confer mismatch repair defects but do not affect nonhomologous-tail removal during recombination. Mol. Cell. Biol., 19(11), 7558-7567.
60. Amin, N. S., Nguyen, M.-N., Oh, S. and Kolodner, R. D. (2001) exo-1 dependent mutator mutations: model systems for studying functional interactions in mismatch repair. Mol. Cell. Biol., 21, 5142-5155.
61. Sia, E. A., Dominska, M., Stefanovic, L. and Petes, T. D. (2001) Isolation and characterization of point mutations in mismatch repair genes that destabilize microsatellites in yeast. Mol. Cell. Biol., 21(23), 8157-8167.
62. Argueso, J. L., Smith, D., Yi, J., Waase, M., Sarin, S. and Alani, E. (2002) Analysis of conditional mutations in the Saccharomyces cerevisiae MLH1 gene in mismatch repair and in meiotic crossing over. Genetics, 160, 909-921.
63. Shimodaira, H., Filosi, N., Shibata, H., Suzuki, T., Radice, P., Kanamaru, R., Friend, S. H., Kolodner, R. D. and Ishioka, C. (1998) Functional analysis of human MLH1 mutations in Saccharomyces cerevisiae. Nature Genet., 19, 384-389.
64. Clark, A. B., Cook, M. E., Tran, H. T., Gordenin, D. A., Resnick, M. and Kunkel, T. A. (1999) Functional analysis of human MutSα and MutSβ complexes in yeast. Nucleic Acids Res., 27(3), 736-742.
65. Andreutti-Zaugg, C., Scott, R. J. and Iggo, R. (1997) Inhibition of nonsense-mediated messenger RNA decay in clinical samples facilitates detection of human MSH2 mutations with an in vivo fusion protein assay and conventional techniques. Cancer Res., 57, 3288-3293.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the invention.

TABLE 1

Classification of human DNA mismatch repair
protein variants for cancer presusceptibility
testing*
MLH1
Human MLH1 proteins conferring upon an
individual a greater than normal suscep-
tibility to develop cancer

| | |
|---|---|
| 23D | (SEQ ID NO: 262) |
| 29I | (SEQ ID NO: 263) |
| 38T | (SEQ ID NO: 264) |
| 40F | (SEQ ID NO: 265) |
| 40N | (SEQ ID NO: 266) |
| 40T | (SEQ ID NO: 267) |
| 41E | (SEQ ID NO: 268) |
| 41G | (SEQ ID NO: 269) |
| 41N | (SEQ ID NO: 270) |
| 42E | (SEQ ID NO: 271) |
| 42T | (SEQ ID NO: 272) |
| 42V | (SEQ ID NO: 273) |
| 43A | (SEQ ID NO: 274) |
| 43D | (SEQ ID NO: 275) |
| 43E | (SEQ ID NO: 276) |
| 43F | (SEQ ID NO: 277) |
| 43H | (SEQ ID NO: 278) |
| 43I | (SEQ ID NO: 279) |
| 43L | (SEQ ID NO: 280) |
| 43M | (SEQ ID NO: 281) |
| 43P | (SEQ ID NO: 282) |
| 43S | (SEQ ID NO: 283) |
| 43T | (SEQ ID NO: 284) |
| 43V | (SEQ ID NO: 285) |
| 43W | (SEQ ID NO: 286) |
| 43Y | (SEQ ID NO: 287) |
| 44D | (SEQ ID NO: 288) |
| 44G | (SEQ ID NO: 289) |
| 44K | (SEQ ID NO: 290) |
| 44M | (SEQ ID NO: 291) |
| 44N | (SEQ ID NO: 292) |
| 45I | (SEQ ID NO: 293) |
| 46T | (SEQ ID NO: 294) |
| 47S | (SEQ ID NO: 295) |
| 47T | (SEQ ID NO: 296) |
| 48G | (SEQ ID NO: 297) |
| 48Y | (SEQ ID NO: 298) |
| 49E | (SEQ ID NO: 299) |
| 49M | (SEQ ID NO: 300) |
| 49N | (SEQ ID NO: 301) |
| 51A | (SEQ ID NO: 302) |
| 51D | (SEQ ID NO: 303) |
| 55S | (SEQ ID NO: 304) |
| 56M | (SEQ ID NO: 305) |
| 56P | (SEQ ID NO: 306) |
| 57N | (SEQ ID NO: 307) |
| 59F | (SEQ ID NO: 308) |
| 59H | (SEQ ID NO: 309) |
| 59N | (SEQ ID NO: 310) |
| 59T | (SEQ ID NO: 311) |
| 61N | (SEQ ID NO: 312) |
| 63G | (SEQ ID NO: 313) |
| 63Y | (SEQ ID NO: 314) |
| 64I | (SEQ ID NO: 315) |
| 64S | (SEQ ID NO: 316) |
| 65A | (SEQ ID NO: 317) |
| 65D | (SEQ ID NO: 318) |
| 65E | (SEQ ID NO: 319) |
| 65S | (SEQ ID NO: 320) |
| 65V | (SEQ ID NO: 321) |
| 67W | (SEQ ID NO: 322) |
| 68F | (SEQ ID NO: 323) |
| 68N | (SEQ ID NO: 324) |
| 68S | (SEQ ID NO: 325) |
| 70I | (SEQ ID NO: 326) |
| 70N | (SEQ ID NO: 327) |
| 72G | (SEQ ID NO: 328) |
| 73M | (SEQ ID NO: 329) |
| 73P | (SEQ ID NO: 330) |
| 74L | (SEQ ID NO: 331) |
| 76E | (SEQ ID NO: 332) |
| 77S | (SEQ ID NO: 333) |
| 77Y | (SEQ ID NO: 334) |
| 79W | (SEQ ID NO: 335) |
| 80I | (SEQ ID NO: 336) |
| 80S | (SEQ ID NO: 337) |
| 80V | (SEQ ID NO: 338) |
| 82K | (SEQ ID NO: 339) |
| 82M | (SEQ ID NO: 340) |
| 82S | (SEQ ID NO: 341) |
| 83F | (SEQ ID NO: 342) |
| 83P | (SEQ ID NO: 343) |
| 89G | (SEQ ID NO: 344) |
| 89V | (SEQ ID NO: 345) |
| 91V | (SEQ ID NO: 346) |
| 99I | (SEQ ID NO: 347) |
| 99L | (SEQ ID NO: 348) |
| 100P | (SEQ ID NO: 349) |
| 100Q | (SEQ ID NO: 350) |
| 101D | (SEQ ID NO: 351) |
| 102D | (SEQ ID NO: 352) |
| 102G | (SEQ ID NO: 353) |
| 103T | (SEQ ID NO: 354) |
| 103V | (SEQ ID NO: 355) |
| 111P | (SEQ ID NO: 356) |
| 111T | (SEQ ID NO: 357) |
| 113A | (SEQ ID NO: 358) |
| 114I | (SEQ ID NO: 359) |
| 115E | (SEQ ID NO: 360) |
| 115F | (SEQ ID NO: 361) |
| 115N | (SEQ ID NO: 362) |
| 115S | (SEQ ID NO: 363) |
| 116A | (SEQ ID NO: 364) |
| 118N | (SEQ ID NO: 365) |
| 128P | (SEQ ID NO: 366) |
| 182G | (SEQ ID NO: 367) |
| 193P | (SEQ ID NO: 368) |
| 304V | (SEQ ID NO: 601) |
| 542P | (SEQ ID NO: 369) |
| 549P | (SEQ ID NO: 370) |
| 640S | (SEQ ID NO: 602) |
| 663G | (SEQ ID NO: 371) |
| 755S | (SEQ ID NO: 372) |

Human MLH1 proteins conferring upon an
individual no greater susceptibility to
develop cancer

| | |
|---|---|
| 22A | (SEQ ID NO: 598) |
| 29S | (SEQ ID NO: 373) |
| 32V | (SEQ ID NO: 374) |
| 36L | (SEQ ID NO: 375) |
| 43C | (SEQ ID NO: 376) |
| 43G | (SEQ ID NO: 377) |
| 43N | (SEQ ID NO: 378) |
| 43Q | (SEQ ID NO: 379) |
| 43R | (SEQ ID NO: 380) |
| 62R | (SEQ ID NO: 381) |
| 64D | (SEQ ID NO: 382) |
| 71D | (SEQ ID NO: 383) |
| 75T | (SEQ ID NO: 384) |
| 95T | (SEQ ID NO: 385) |
| 136S | (SEQ ID NO: 386) |
| 141R | (SEQ ID NO: 599) |
| 160V | (SEQ ID NO: 387) |
| 272V | (SEQ ID NO: 388) |
| 286Q | (SEQ ID NO: 600) |
| 441T | (SEQ ID NO: 389) |
| 648L | (SEQ ID NO: 390) |
| 659Q | (SEQ ID NO: 391) |

MSH2
Human MSH2 proteins conferring upon an
individual a greater than normal suscep-
tibility to develop cancer

| | |
|---|---|
| 100/101-del | (SEQ ID NO: 604) |
| 198G | (SEQ ID NO: 392) |
| 199R | (SEQ ID NO: 400) |
| 272V | (SEQ ID NO: 393) |
| 333R | (SEQ ID NO: 90) |
| 338R | (SEQ ID NO: 607) |
| 439-del | (SEQ ID NO: 609) |
| 440P | (SEQ ID NO: 610) |
| 503P | (SEQ ID NO: 394) |
| 534C | (SEQ ID NO: 611) |
| 595R | (SEQ ID NO: 614) |
| 603N | (SEQ ID NO: 615) |
| 622T | (SEQ ID NO: 616) |
| 636P | (SEQ ID NO: 99) |
| 639R | (SEQ ID NO: 93) |
| 683R | (SEQ ID NO: 395) |
| 692R | (SEQ ID NO: 95) |
| 697R | (SEQ ID NO: 96) |
| 751R | (SEQ ID NO: 97) |

Human MSH2 proteins conferring upon an
individual no greater susceptibility to
develop cancer

| | |
|---|---|
| 30L | (SEQ ID NO: 603) |
| 44M | (SEQ ID NO: 396) |
| 61P | (SEQ ID NO: 397) |
| 127S | (SEQ ID NO: 398) |
| 167H | (SEQ ID NO: 399) |
| 186S | (SEQ ID NO: 89) |
| 199W | (SEQ ID NO: 605) |
| 322V | (SEQ ID NO: 606) |
| 323C | (SEQ ID NO: 401) |
| 333Y | (SEQ ID NO: 91) |
| 349L | (SEQ ID NO: 608) |
| 390F | (SEQ ID NO: 402) |
| 390V | (SEQ ID NO: 403) |
| 562V | (SEQ ID NO: 612) |
| 583S | (SEQ ID NO: 613) |
| 609V | (SEQ ID NO: 92) |
| 647K | (SEQ ID NO: 100) |
| 656H | (SEQ ID NO: 101) |
| 683V | (SEQ ID NO: 404) |
| 688I | (SEQ ID NO: 405) |
| 691T | (SEQ ID NO: 94) |
| 722I | (SEQ ID NO: 617) |
| 729V | (SEQ ID NO: 102) |
| 735V | (SEQ ID NO: 406) |
| 770V | (SEQ ID NO: 98) |
| 845E | (SEQ ID NO: 407) |

*entries refer to human MLH1 or MSH2 proteins having the indicated amino acid residue (single letter code, see abbreviations) at the indicated position. Numbering begins with the methionine encoded by codon 1 (start codon, ATG).

TABLE 2
MLH1 variants examined and oligonucleotides used for making site-directed mutations

| | | Source of | Equivalent | | Restriction |
|---|---|---|---|---|---|
| Gene product | | human variant | substitution | Oligonucleotides used^{d} | site |
| and variant amino acid | | (Reference)^{a,b,c} | in yMLH1 | [(s), sense and (a), anti-sense strand] | alteration^{e} |
| hMLH1 | E23D | (ICG) | E20D | SEQ ID NO: 1 (s) | +EcoRV |
| | | | | SEQ ID NO: 2 (a) | |
| | G67W | (HGMD) | G64W | SEQ ID NO: 3 (s) | +BamHI |
| | | | | SEQ ID NO: 4 (a) | |
| | C77Y | (ICG) | C74Y | SEQ ID NO: 5 (s) | +SspI |
| | | | | SEQ ID NO: 6 (a) | |
| | F80V | (HGMD) | F77V | SEQ ID NO: 148 (s) | −AatII |
| | | | | SEQ ID NO: 149 (a) | |
| | R100P | (ICG) | R97P | SEQ ID NO: 7 (s) | +SmaI |
| | | | | SEQ ID NO: 8 (a) | |
| | E102D | (ICG) | E99D | SEQ ID NO: 9 (s) | −HindIII |
| | | | | SEQ ID NO: 10 (a) | |
| | R182G | (HGMD) | R179G | SEQ ID NO: 11 (s) | None^{f} |
| | | | | SEQ ID NO: 12 (a) | |
| | S193P | (ICG) | S190P | SEQ ID NO: 13 (s) | None^{f} |
| | | | | SEQ ID NO: 14 (a) | |
| | L272V | (ICG) | L272V | SEQ ID NO: 15 (s) | +BstBI |
| | | | | SEQ ID NO: 16 (a) | |
| | A441T | (ICG) | A444T | SEQ ID NO: 17 (s) | None^{f} |
| | | | | SEQ ID NO: 18 (a) | |
| | Q542P | (Terdiman, 2001) | Q552P | SEQ ID NO: 19 (s) | +BspHI |
| | | | | SEQ ID NO: 20 (a) | |
| | L549P | (ICG) | L559P | SEQ ID NO: 21 (s) | +ClaI |
| | | | | SEQ ID NO: 22 (a) | |
| | P648L | (HGMD) | P661L | SEQ ID NO: 150 (s) | +SpeI |
| | | | | SEQ ID NO: 151 (a) | |
| | R659Q | (ICG) | R672Q | SEQ ID NO: 23 (s) | +PvuII |
| | | | | SEQ ID NO: 24 (a) | |
| | E663G | (ICG) | E676G | SEQ ID NO: 25 (s) | +SalI |
| | | | | SEQ ID NO: 26 (a) | |
| | R755S | (Syngal, 1999) | R768S | SEQ ID NO: 27 (s) | +BstBI |
| | | | | SEQ ID NO: 28 (a) | |
| MLH1_h | A29S | (ICG) | — | SEQ ID NO: 152 (s) | +EagI |
| (1-86) | | | | SEQ ID NO: 153 (a) | |
| | I32V | (GeneSnP) | — | SEQ ID NO: 154 (s) | +EagI |
| | | | | SEQ ID NO: 155 (a) | |
| MLH1_h | A128P | (ICG) | — | SEQ ID NO: 156 (s) | +StuI |
| (77-177) | | | | SEQ ID NO: 157 (a) | |
| | A160V | (ICG) | — | SEQ ID NO: 158 (s) | −HindIII |
| | | | | SEQ ID NO: 159 (a) | |

^{a}ICG, variant reported on-line in the database of the International Collaborative Group on Hereditary Nonpolyposis Colorectal Cancer (http://www.nfdht.nl)
^{b}HGMD, variant reported on-line in the Human Gene Mutation Database (http://www.hgmd.org)
^{c}GeneSnP, variant reported on-line in the GeneSnP database (http://www.genome.utah.edu/genesnps/)
^{d}Oligonucleotides with the indicated sequence were used for making site-directed mutations in the indicated MMR gene as described in Examples 1 and 3.
^{e}The restriction site alterations are silent at the amino acid sequence level, except for the indicated substitution. +, restriction site additon; −, restriction site loss.
^{f}Alteration screened by DNA sequencing.

TABLE 3
Functional consequence of amino acid
substitutions in yeast MLH1p

| | Mutation | Relative |
|---|---|---|
| | frequency × 10⁻⁵ | mutation defect |
| MLH1 Variant | (95% CI) | (range) |
| None | 265 (191-338) | 189 (136-241) |
| MLH1 wildtype | 1.4 (1.2-1.6) | 1.0 (0.8-1.1) |
| G19A | 0.8 | 0.6 |
| E20D | 95.2* | 68 |
| G64W | 315** | 225 |
| C74Y | 83.1* | 59 |
| F77V | 77.9* | 56 |
| R97P | 168* | 120 |
| E99D | 10.8* | 7.7 |
| P138R | 0.7 | 0.5 |
| R179G | 1.9* | 1.4 |
| S190P | 445** | 318 |
| L272V | 0.9 | 0.6 |
| K286Q | 1.6 | 1.1 |
| D304V | 348** | 248 |
| A444T | 1.4 | 1.0 |
| Q552P | 32.6* | 23 |
| R559P | 40.2* | 29 |
| P653S | 15.8* | 11 |
| P661L | 1.8 | 1.3 |
| R672Q | 0.7 | 0.5 |
| E676G | 7.1* | 5.1 |
| R768S | 171** | 122 |

Mutation frequencies, 95% confidence interval (CI), mutation defects and statistical comparisons were determined as described in Example 1. Values are from six independent experiments.
**denotes significantly greater than wild-type MLH1 and significantly greater or not different than “None”.
*denotes significantly greater than wild-type MLH1 and significantly less than “None”. These conclusions were based on comparisons to control values within each independent experiment.

TABLE 4
Functional analysis of human-yeast hybrid MLH1 genes

| | Mutation | |
|---|---|---|
| Gene | frequency × 10⁻⁵ | Mutation defect |
| None (pMETc vector) | 174; 303 | 144; 193 |
| MLH1 | 1.2; 1.6 | 1.0 |
| MLH1_h(175-267) | 30.8 | 25.4 |
| MLH1_h(175-214) | 89.4 | 56.9 |
| MLH1_h(208-267) | 5.7 | 3.7 |
| MLH1_h(265-341) | 48.3 | 40.0 |
| MLH1_h(265-311) | 35.7 | 22.8 |
| MLH1_h(298-341) | 38.4 | 24.5 |

TABLE 5
Functional analysis of amino acid substitutions
in hybrid human-yeast MLH1 genes

| | | |
|---|---|---|
| | Mutation | |
| Experiment/Gene | frequency × 10⁻⁵ | Mutation defect |

Experiment #1

| | | |
|---|---|---|
| None (pMETc) | 274 | 12 |
| MLH1_h(1-86) | 23.3 | 1.0 |
| MLH1_h(1-86) A29S | 33.0 | 1.4 |
| MLH1_h(1-86) I32V | 32.6 | 1.4 |

Experiment #2

| | | |
|---|---|---|
| None (pMETc) | 234 | 8.4 |
| MLH1_h(41-86) | 27.8 | 1.0 |
| MLH1_h(41-86) G67E | 153* | 5.5 |

Experiment #3

| | | |
|---|---|---|
| None (pMETc) | 182 | 16 |
| MLH1_h(77-134) | 11.5 | 1.0 |
| MLH1_h(77-134) N35S | 214** | 19 |
| MLH1_h(77-134) C77R | 290** | 25 |

Experiment #4

| | | |
|---|---|---|
| None (pMETc) | 419 | 64 |
| MLH1_h(77-177) | 6.6 | 1.0 |
| MLH1_h(77-177) A128P | 228* | 35 |
| MLH1_h(77-177) A160V | 5.9 | 0.9 |

Mutation frequencies, mutation defects and statistical comparisons were determined as described in Example 3. Values are from four independent experiments.
**denotes significantly greater than the appropriate control hybrid MLH1 gene and significantly greater or not different than “None”.
*denotes significantly greater than the appropriate control hybrid MLH1 gene and significantly less than “None”.

TABLE 6
MSH2 variants examined and oligonucleotides used for making site-directed mutations

| | | Source of | Equivalent | | Restriction |
|---|---|---|---|---|---|
| Gene product | | human variant | substitution | Oligonucleotides used^{e} | site |
| and variant amino acid | | (Reference)^{a,b,c,d} | in yMSH2 | [(s), sense and (a), anti-sense strand] | alteration^{f} |
| hMSH2 | T44M | (HGMD) | T44M | SEQ ID NO: 258 (s) | None^{g} |
| | | | | SEQ ID NO: 259 (a) | |
| | Q61P | (ICG) | Q61P | SEQ ID NO: 168 (s) | −DraI |
| | | | | SEQ ID NO: 169 (a) | |
| | N127S | (ICG) | N123S | SEQ ID NO: 170 (s) | +BamHI |
| | | | | SEQ ID NO: 171 (a) | |
| | D167H | (HGMD) | D163H | SEQ ID NO: 172 (s) | +BtgI |
| | | | | SEQ ID NO: 173 (a) | |
| | N186S | (Samowitz, 2001) | N182S | SEQ ID NO: 47 (s) | +XbaI |
| | | | | SEQ ID NO: 48 (a) | |
| | E198G | (ICG) | E194G | SEQ ID NO: 174 (s) | −BsgI |
| | | | | SEQ ID NO: 175 (a) | |
| | C199R | (ICG) | C195R | SEQ ID NO: 176 (s) | −BsgI |
| | | | | SEQ ID NO: 177 (a) | |
| | A272V | (Syngal, 1999) | A267V | SEQ ID NO: 178 (s) | −NsiI |
| | | | | SEQ ID NO: 179 (a) | |
| | S323C | (HGMD) | S318C | SEQ ID NO: 260 (s) | None^{g} |
| | | | | SEQ ID NO: 261 (a) | |
| | C333R | (ICG) | C345R | SEQ ID NO: 49 (s) | +BsaMI |
| | | | | SEQ ID NO: 50 (a) | |
| | C333Y | (ICG) | C345Y | SEQ ID NO: 51 (s) | +BsaMI |
| | | | | SEQ ID NO: 52 (a) | |
| | L390F | (HGMD) | L402F | SEQ ID NO: 180 (s) | +BstBI |
| | | | | SEQ ID NO: 181 (a) | |
| | L390V | (Guerrette, 1998) | L402V | SEQ ID NO: 182 (s) | +BstBI |
| | | | | SEQ ID NO: 183 (a) | |
| | L503P | (ICG) | L521P | SEQ ID NO: 184 (s) | −BglII |
| | | | | SEQ ID NO: 185 (a) | |
| | A609V | (Holinski-Feder, 2001) | A627V | SEQ ID NO: 53 (s) | +BsrGI |
| | | | | SEQ ID NO: 54 (a) | |
| | H639R | (HGMD) | H658R | SEQ ID NO: 55 (s) | +AatII |
| | | | | SEQ ID NO: 56 (a) | |
| | G683R | (Samowitz, 2001) | G702R | SEQ ID NO: 186 (s) | +EcoRV |
| | | | | SEQ ID NO: 187 (a) | |
| | G683V | (Samowitz, 2001) | G702V | SEQ ID NO: 188 (s) | +EcoRV |
| | | | | SEQ ID NO: 189 (a) | |
| | M688I | (HGMD) | M707I | SEQ ID NO: 190 (s) | +VspI |
| | | | | SEQ ID NO: 191 (a) | |
| | I691T | (Samowitz, 2001) | I710T | SEQ ID NO: 57 (s) | +AgeI |
| | | | | SEQ ID NO: 58 (a) | |
| | G692R | (HGMD) | G711R | SEQ ID NO: 59 (s) | +MspA1 |
| | | | | SEQ ID NO: 60 (a) | |
| | C697R | (HGMD) | C716R | SEQ ID NO: 61 (s) | +XhoI |
| | | | | SEQ ID NO: 62 (a) | |
| | I735V | (egSNP) | I754V | SEQ ID NO: 192 (s) | +AflII |
| | | | | SEQ ID NO: 193 (a) | |
| | G751R | (ICG) | G770R | SEQ ID NO: 63 (s) | +SacI |
| | | | | SEQ ID NO: 64 (a) | • |
| | I770V | (Swiss Prot) | I789V | SEQ ID NO: 65 (s) | +NruI |
| | | | | SEQ ID NO: 66 (a) | |
| | K845E | (HGMD) | K873E | SEQ ID NO: 194 (s) | +EaeI |
| | | | | SEQ ID NO: 195 (a) | |
| MSH2_h | A636P | (ICG) | — | SEQ ID NO: 67 (s) | +XbaI |
| (621-739) | | | | SEQ ID NO: 68 (a) | |
| | E647K | (HGMD) | — | SEQ ID NO: 69 (s) | None^{g} |
| | | | | SEQ ID NO: 70 (a) | |
| | Y656H | (Nakahara, 1997) | — | SEQ ID NO: 71 (s) | +AatII |
| | | | | SEQ ID NO: 72 (a) | |
| | M729V | (Nakahara, 1997) | — | SEQ ID NO: 73 (s) | +EaeI |
| | | | | SEQ ID NO: 74 (a) | |

^{a}HGMD, variant reported on-line in the Human Gene Mutation Database (http://uwcmmlls.uwcm.ac.uk)
^{b}ICG, variant reported on-line in the database of the International Collaborative Group on Hereditary Nonpolyposis Colorectal Cancer (http://www.nfdht.nl)
^{c}egSnP, variant reported on-line in the egSnP database (http://www.dir-apps.niehs.nih.gov/egsnp/home.htm)
^{d}Swiss-Prot, variant reported on-line in the Swiss-Prot database (http://us.expasy.org)
^{e}Sense and antisense oligonucleotides were used for making site-directed mutations in the indicated MMR genes as described in Example 4.
^{f}The restriction site alterations are silent at the amino acid sequence level, except for the indicated substitution. +, restriction site additon; −, restriction site loss.
^{g}Alteration screened by DNA sequencing.

TABLE 6a
Additional MLH1 and MSH2 variants examined and oligonucleotides
used for making site-directed mutations

| | | Source of | Equivalent | | Restriction |
|---|---|---|---|---|---|
| Gene product | | human variant | substitution | Oligonucleotides used^{c} | site |
| and variant amino acid | | (Reference)^{a,b} | in yMSH2 | [(s), sense and (a), anti-sense strand] | alteration^{d} |
| hMLH1 | G22A | (Woods, 2003) | G19A | SEQ ID NO: 538 (s) | +PvuII |
| | | | | SEQ ID NO: 539 (a) | |
| | P141R | (Giraldo, 2003) | P138R | SEQ ID NO: 540 (s) | +MluI |
| | | | | SEQ ID NO: 541 (a) | |
| | K286Q | (Beck, 1997) | K286Q | SEQ ID NO: 542 (s) | +Bsu36I |
| | | | | SEQ ID NO: 543 (a) | |
| | D304V | (HGMD) | D304V | SEQ ID NO: 44 (s) | +EagI |
| | | | | SEQ ID NO: 545 (a) | |
| | P640S | (Giraldo, 2003) | P653S | SEQ ID NO: 546 (s) | +BlpI |
| | | | | SEQ ID NO: 547 (a) | |
| hMSH2 | P30L | (Nafa, 2003) | P30L | SEQ ID NO: 548 (s) | +HindIII |
| | | | | SEQ ID NO: 549 (a) | |
| | VE100/101del | (ICG) | VE106/107del | SEQ ID NO: 550 (s) | +BglII |
| | | | | SEQ ID NO: 551 (a) | |
| | C199W | (Nafa, 2003) | C195W | SEQ ID NO: 552 (s) | −BsgI |
| | | | | SEQ ID NO: 553 (a) | |
| | G322V | (Otway, 2000) | G317V | SEQ ID NO: 554 (s) | None^{e} |
| | | | | SEQ ID NO: 555 (a) | |
| | G338R | (ICG) | G350R | SEQ ID NO: 556 (s) | +XhoI |
| | | | | SEQ ID NO: 557 (a) | |
| | P349L | (Nafa, 2003) | P361L | SEQ ID NO: 558 (s) | +PvuII |
| | | | | SEQ ID NO: 559 (a) | |
| | P439-del | (Jeong, 2003) | P456-del | SEQ ID NO: 560 (s) | +AflII |
| | | | | SEQ ID NO: 561 (a) | |
| | L440P | (ICG) | L457P | SEQ ID NO: 562 (s) | None^{e} |
| | | | | SEQ ID NO: 563 (a) | |
| | R534C | (Gorlov, 2003) | R552C | SEQ ID NO: 564 (s) | +SacI |
| | | | | SEQ ID NO: 565 (a) | |
| | E562V | (HGMD) | E580V | SEQ ID NO: 566 (s) | +NruI |
| | | | | SEQ ID NO: 567 (a) | |
| | N583S | (Wagner, 2003) | N601S | SEQ ID NO: 568 (s) | +ClaI |
| | | | | SEQ ID NO: 569 (a) | |
| | L595R | (Nafa, 2003) | L613R | SEQ ID NO: 570 (s) | +BglII |
| | | | | SEQ ID NO: 571 (a) | |
| | D603N | (ICG) | D621N | SEQ ID NO: 572 (s) | +BsrDI |
| | | | | SEQ ID NO: 573 (a) | |
| | P622T | (ICG) | P640T | SEQ ID NO: 574 (s) | +BstBI |
| | | | | SEQ ID NO: 575 (a) | |
| | V722I | (Gorlov, 2003) | V741I | SEQ ID NO: 576 (s) | +EcoRI |
| | | | | SEQ ID NO: 577 (a) | |

^{a}HGMD, variant reported on-line in the Human Gene Mutation Database (http://www.hgmd.org)
^{b}ICG, variant reported on-line in the database of the International Collaborative Group on Hereditary Nonpolyposis Colorectal Cancer (http://www.nfdht.nl)
^{c}Sense and antisense oligonucleotides were used for making site-directed mutations in the indicated MMR genes as described in Example 4.
^{d}The restriction site alterations are silent at the amino acid sequence level, except for the indicated substitution. +, restriction site additon; −, restriction site loss.
^{e}Alteration screened by DNA sequencing.

TABLE 7
The functional consequence of amino acid substitutions in yeast MSH2p

| | (GT)₁₆G::URA3 | | CAN1 | |
|---|---|---|---|---|
| | Mutation | Relative | Mutation | Relative |
| | frequency × 10⁻⁵ | mutation defect | frequency × 10⁻⁷ | mutation defect |
| MSH2 Variant | (95% CI) | (range) | (95% CI) | (range) |
| None | 350 (270-430) | 88 (68-108) | 270 (230-320) | 28 (23-33) |
| MSH2 wildtype | 4.0 (2.1-5.9) | 1.0 (0.5-1.5) | 9.8 (7.4-12) | 1.0 (0.8-1.2) |
| P30L | 4.1 | 1.0 | 21 | 2.1 |
| T44M | 4.9 | 1.2 | 6.7 | 0.7 |
| Q61P | 5.4 | 1.4 | 11 | 1.1 |
| VE-106/107-del | 274* | 68 | 172 | 18 |
| N123S | 3.0 | 0.8 | 5.4 | 0.6 |
| D163H | 3.2 | 0.8 | 4.4 | 0.4 |
| N182S | 2.9 | 0.7 | 12 | 1.2 |
| E194G | 50* | 13 | 12 | 1.2 |
| C195R | 280** | 70 | 360 | 37 |
| C195W | 1.8 | 0.4 | 8.3 | 0.8 |
| A267V | 4.7* | 1.2 | 7.4 | 0.8 |
| G317V | 1.8 | 0.4 | 22 | 2.2 |
| S318C | 6.3 | 1.6 | 6.5 | 0.7 |
| C345R | 9.7* | 2.4 | 21 | 2.1 |
| C345Y | 8.0 | 2.0 | 18 | 1.8 |
| G350R | 336** | 84 | 319 | 32 |
| P361L | 2.6 | 0.6 | 17 | 1.8 |
| L402F | 0.6 | 0.2 | 6.6 | 0.7 |
| L402V | 0.9 | 0.2 | 9.9 | 1.0 |
| P456-del | 85.8* | 21 | 14 | 1.4 |
| L457P | 138* | 34 | 55 | 5.6 |
| L521P | 0.9 | 0.2 | 140 | 14 |
| R552C | 11.5* | 2.9 | 35 | 3.6 |
| E580V | 3.5 | 0.9 | 8.2 | 0.8 |
| N601S | 2.8 | 0.7 | 4.6 | 0.5 |
| L613R | 6.7* | 1.7 | 8.4 | 0.8 |
| D621N | 21.3* | 5.3 | 11 | 1.2 |
| A627V | 3.9 | 1.0 | 17 | 1.7 |
| P640T | 9.8* | 2.4 | 11.9 | 1.2 |
| H658R | 410** | 103 | 270 | 28 |
| G702R | 410** | 103 | 220 | 22 |
| G702V | 2.4 | 0.6 | 11 | 1.1 |
| M707I | 3.4 | 0.9 | 7.2 | 0.7 |
| I710T | 1.9 | 0.5 | 17 | 1.7 |
| G711R | 280* | 70 | 320 | 33 |
| C716R | 350** | 88 | 440 | 45 |
| V741I | 3.4 | 0.8 | 18 | 1.8 |
| I754V | 4.1 | 1.0 | 6.8 | 0.7 |
| G770R | 350** | 88 | 350 | 36 |
| I789V | 2.8 | 0.7 | 14 | 1.4 |
| K873E | 1.4 | 0.4 | 5.3 | 0.5 |

Mutation frequencies, 95% confidence intervals (CI), mutation defects and statistical comparisons were determined as described in Example 3. Mean mutation frequencies for the (GT)₁₆G::URA3 reporter gene are from six independent experiments.
**denotes signficantly greater than wild-type MSH2 and significantly greater or not different than “None” (i.e., inactivating mutation).
*denotes signficantly greater than wild-type MSH2 and significantly less than “None” (i.e., efficiency polymorphism). These conclusions were based on comparisons to control values within each independent experiment. Median mutation frequencies for the CAN1-based fluctuation test are from seven independent experiments.

TABLE 8
Functional analysis of human-yeast hybrid MSH2 genes
and hybrid MSH2 genes containing codon alterations

| | | |
|---|---|---|
| | Mutation | Relative |
| Gene | frequency × 10⁻⁵ | mutation defect |

Experiment #1

| | | |
|---|---|---|
| None (pMETc vector) | 328 | 199 |
| MSH2 | 1.6 | 1.0 |
| MSH2_h(1-63) | 245 | 148 |
| MSH2_h(621-832) | 228 | 138 |
| MSH2_h(621-739) | 3.2 | 1.9 |
| MSH2_h(730-832) | 245 | 148 |

Experiment #2

| | | |
|---|---|---|
| None (pMETc vector) | 424 | 230 |
| MSH2 | 1.8 | 1.0 |
| MSH2_h(621-832) | 155 | 84 |
| MSH2_h(621-832)ins9 | 22 | 12 |
| MSH2_h(730-832) | 369 | 201 |
| MSH2_h(730-832)ins9 | 18 | 10 |

Experiment #3

| | | |
|---|---|---|
| None (pMETc vector) | 258 | 30 |
| MSH2_h(621-739) | 8.5 | 1.0 |
| MSH2_h(621-739) A636P | 239** | 28 |
| MSH2_h(621-739) E647K | 8.9 | 1.0 |
| MSH2_h(621-739) Y656H | 5.5 | 0.6 |
| MSH2_h(621-739) M729V | 12 | 1.4 |

Mutation frequencies, mutation defects and statistical comparisons were determined as described in Example 1.
**denotes significantly greater than MSH2_h(621-739) and not signficantly different than “None” (i.e. inactivating mutation) based on comparisons within this experiment.

TABLE 9
Colorimetric analysis of yeast strains YBT39, YBT40 and YBT41

| Strain | Expression vector | White colonies | Pink colonies |
|---|---|---|---|
| YBT39 | pMETc | 25-50% | 50-75% |
| YBT39 | pMETc/MSH2 | 100% | 0% |
| YBT40 | pMETc | 40-60% | 30-40% |
| YBT40 | pMLH1 | 100% | 0% |
| YBT41 | pMETc | 25-50% | 50-75% |
| YBT41 | pMLH1 | 100% | 0% |

TABLE 10
Functional analysis of human-yeast hybrid MLH1 genes

| | ADE2::MS3::ADE2^{a} | | | | CAN1^{b} | (GT)₁₆G::URA3^{c} |
|---|---|---|---|---|---|---|
| | Total | Sectored CFU | | White CFU | Mutation frequency | Mutation frequency |
| MLH1 gene | CFU | (% of total) | | (% of total) | (mutation defect) | (mutation defect) |
| None | 1226 | 1224 | (>95%) | 2 (<5%) | 3.1 × 10⁻⁵ (44) | 1.9 × 10⁻³ (75) |
| MLH1 | 452 | 2 | (<2%) | 450 (>98%) | 7.1 × 10⁻⁷ (1) | 2.5 × 10⁻⁵ (1) |
| MLH1_h(41-86) | 180 | 26 | (14) | 154 (86%) | 2.8 × 10⁻⁶ (4.0) | 1.2 × 10⁻⁴ (4.8) |
| MLH1_h(77-134) | 515 | 3 | (<2%) | 512 (>98%) | 1.7 × 10⁻⁶ (2.4) | 5.4 × 10⁻⁵ (2.1) |
| ^{a}Yeast strain YBT41, which contains an MLH1 deletion and the ADE2::MS3::ADE2 allele, was transformed with expression vectors carrying the indicated MLH1 gene or the parental expression | | | | | | |
| # vector pMETc lacking an MLH1 gene (“None”) and cells were plated on SD medium lacking histidine and containing 4 μg/ml adenine. Colonies (CFU), which are transformants since they grow | | | | | | |
| # without added histidine, were counted and visually inspected for red-white sectoring. In all transformations a | | | | | | |
| # background of ≈10% red colonies was consistently observed (see FIG. 1B) and these colonies were excluded from our analysis. The origin of these colonies are | | | | | | |
| # presumably host cells in which the ADE2 gene had mutated prior to, or shortly after, the transformation. | | | | | | |
| ^{b}Mutation frequencies were based on forward mutation to canavanine resistance and were determined for the MLH1-deletion strain YBT24 harboring the | | | | | | |
| # indicated MLH1 gene or the parental expression vector pMETc (“None”). The median value of 9 independent cultures is shown. Mutation defects were calculated with respect to the mutation frequency conferred by the wild-type MLH1 gene. | | | | | | |

^{c}Mutation frequencies were determined using a URA3 reporter gene preceded by an in-frame (GT)₁₆G microsatellite. Values are from Ellison et al. (2001).

TABLE 11
Termination codons identified in hybrid human-yeast MLH1 genes

| | MLH1 codon | | | | | |
|---|---|---|---|---|---|---|
| | (species/region | | Screening | Codon | | Number of times |
| Hybrid gene | of hybrid)^{a} | | method^{b} | alteration | Consequence | isolated |
| MLH1_h(41-86) | 34 | (yeast) | a | GAG→TAG | E34-Term | 1 |
| | 52 | (human) | a, b, b | AAA→TAA | K52-Term | 3 |
| | 53 | (human) | b, b | GAG→TAG | E53-Term | 2 |
| | 57 | (human) | a | AAG→TAG | K57-Term | 1 |
| | 71 | (human) | a | GAA→TAA | E71-Term | 1 |
| | 77 | (human) | b | TGT→TGA | C77-Term | 1 |
| | 120 | (yeast) | a | AGA→TGA | R120-Term | 1 |
| | 142 | (yeast) | a | AAA→TAA | K142-Term | 1 |
| MLH1_h(77-134) | 77 | (human) | a | CGA→TGA | C77-Term | 1 |
| | 91 | (human) | a | TTA→TAA | L91-Term | 1 |
| | 97 | (human) | a | TAT→TAA | Y97-Term | 1 |
| | 100 | (human) | a | CGA→TGA | R100-Term | 1 |
| | 104 | (human) | a | TTG→TAG | L104-Term | 1 |

^{a}Codon numbering is relative to the yeast or human portion of the hybrid MLH1 proteins as depicted in FIG. 3B.
^{b}Prospective screening methods utilized yeast strain YBT24 for qualitative patch assays (“a”) or YBT41 for a colorimetric assay (“b”) as described in the Materials and Methods section.

TABLE 12
MLH1 missense mutations identified in human-yeast hybrid MLH1_h(41-86)

| | | | | | | |
|---|---|---|---|---|---|---|
| MLH1 gene or | Screening | Missense | | Corresponding | Mutation | |
| variant codon # | method^{a} | mutation | Consequence | human residue | defect^{b} | SEQ ID NO.s |

Yeast codon:

| | | | | | | |
|---|---|---|---|---|---|---|
| 8 | a | CTT→CAC | L8H | L11 | ++ | SEQ ID NO: 408 |
| 16 | a | ATT→TTT | I16F | I19 | ++ | SEQ ID NO: 409 |
| 26 | a | GTA→ATA | V26I | A29 | ++ | SEQ ID NO: 410 |
| 35 | a | AAT→GAT | N35D | N38 | +++ | SEQ ID NO: 411 |
| | a | AAT→ACT | N35T | N38 | ++ | SEQ ID NO: 412 |
| 37 | a, a | ATC→ACC | I37T | L40 | +++ | SEQ ID NO: 413 |
| | b | ATC→AAC | I37N | L40 | +++ | SEQ ID NO: 414 |

Human codon:

| | | | | | | |
|---|---|---|---|---|---|---|
| 41 | a | GAT→GGT | D41G | — | ++ | SEQ ID NO: 415 |
| 42 | a | GCA→ACA | A42T | — | +++ | SEQ ID NO: 416 |
| | b | GCA→GAA | A42E | — | +++ | SEQ ID NO: 417 |
| | b | GCA→GTA | A42V^{c} | — | +++ | SEQ ID NO: 418 |
| 44 | a | TCC→TTC | S44F | — | +++ | SEQ ID NO: 419 |
| 45 | a, b | ACA→ATA | T45I | — | +++ | SEQ ID NO: 420 |
| 46 | a | AGT→ACT | S46T | — | ++ | SEQ ID NO: 421 |
| 47 | b | ATT→ACT | I47T | — | ++ | SEQ ID NO: 422 |
| | a | ATT→AGT | I47S | — | +++ | SEQ ID NO: 423 |
| 48 | a | CAA→TAT | Q48Y | — | +++ | SEQ ID NO: 424 |
| 49 | b | GTG→GAG | V49E | — | ++ | SEQ ID NO: 425 |
| | a | GTG→ATG | V49M | — | ++ | SEQ ID NO: 426 |
| | a | GTG→GCG | V49A | — | +++ | SEQ ID NO: 427 |
| 51 | a, b | GTT→GAT | V51D | — | ++ | SEQ ID NO: 428 |
| | a | GTT→GCT | V51A | — | ++ | SEQ ID NO: 429 |
| 52 | a, a, b | AAA→ATA | K52I | — | + | SEQ ID NO: 430 |
| 53 | a, a, b | GAG→GTG | E53V | — | ++ | SEQ ID NO: 431 |
| 54 | a | GGA→AGA | G54R | — | + | SEQ ID NO: 432 |
| 55 | a, a, b | GGC→GAC | G55D | — | + | SEQ ID NO: 433 |
| | a | GGC→AGC | G55S | — | ++ | SEQ ID NO: 434 |
| 56 | a | CTG→ATG | L56M | — | + | SEQ ID NO: 435 |
| | a | CTG→CCG | L56P | — | +++ | SEQ ID NO: 436 |
| 57 | a | AAG→GAG | K57E^{c} | — | + | SEQ ID NO: 437 |
| | b | AAG→AAC | K57N | — | +++ | SEQ ID NO: 438 |
| 59 | b | ATT→AAT | I59N | — | +++ | SEQ ID NO: 439 |
| | a, a | ATT→TTT | I59F | — | +++ | SEQ ID NO: 440 |
| | a | ATT→ACT | I59T | — | +++ | SEQ ID NO: 441 |
| 60 | a | CAG→CCG | Q60P | — | ++ | SEQ ID NO: 442 |
| 61 | a | ATC→AAC | I61N | — | ++ | SEQ ID NO: 443 |
| 63 | a | GAC→TAC | D63Y | — | +++ | SEQ ID NO: 444 |
| 64 | b | AAT→ATT | N64I | — | ++ | SEQ ID NO: 445 |
| 65 | b | GGC→GTC | G65V | — | +++ | SEQ ID NO: 446 |
| | a | GGC→GCC | G65A | — | +++ | SEQ ID NO: 447 |
| | a | GGC→GAC | G65D | — | ++ | SEQ ID NO: 448 |
| | a | GGC→AGC | G65S | — | ++ | SEQ ID NO: 449 |
| 67 | a, a, b | GGG→GAG | G67E | — | ++ | SEQ ID NO: 450 |
| | a | GGG→GTG | G67V | — | +++ | SEQ ID NO: 451 |
| 68 | a | ATC→AAC | I68N | — | +++ | SEQ ID NO: 452 |
| | a, b | ATC→TTC | I68F | — | ++ | SEQ ID NO: 453 |
| | b | ATC→AGC | I68S^{c} | — | ++ | SEQ ID NO: 454 |
| 70 | a | AAA→AAT | K70N | — | +++ | SEQ ID NO: 455 |
| | a | AAA→ATA | K70I | — | +++ | SEQ ID NO: 456 |
| 72 | a, b | GAT→GGT | D72G | — | ++ | SEQ ID NO: 457 |
| | a, b | GAT→GTT | D72V | — | + | SEQ ID NO: 458 |
| 73 | b | CTG→ATG | L73M | — | ++ | SEQ ID NO: 459 |
| | a | CTG→CCG | L73P | — | ++ | SEQ ID NO: 460 |
| | a | CTG→CAG | L73Q | — | ++ | SEQ ID NO: 461 |
| 76 | b | GTA→GAA | V76E | — | +++ | SEQ ID NO: 462 |
| 77 | a | TGT→GGT | C77G | — | ++ | SEQ ID NO: 463 |
| | b | TGT→TCT | C77S | — | ++ | SEQ ID NO: 464 |
| 79 | b | AGG→TGG | R79W^{c} | — | ++ | SEQ ID NO: 465 |
| 80 | a | TTC→TCC | F80S^{c} | — | ++ | SEQ ID NO: 466 |
| | b | TTC→CTC | F80L | — | +++ | SEQ ID NO: 467 |
| | b | TTC→ATC | F80I | — | +++ | SEQ ID NO: 468 |
| 81 | a | ACG→ATG | T81M | — | + | SEQ ID NO: 469 |
| 82 | a, a | ACG→TCG | T82S | — | + | SEQ ID NO: 470 |
| | a, b | ACG→AAG | T82K | — | +++ | SEQ ID NO: 471 |
| | a | ACG→ATG | T82M | — | +++ | SEQ ID NO: 472 |
| 83 | a, b | TCC→CCC | S83P | — | ++ | SEQ ID NO: 473 |
| | a | TCC→TTC | S83F | — | +++ | SEQ ID NO: 474 |
| 84 | a | AAA→GAA | K84R | — | ++ | SEQ ID NO: 475 |
| | a | AAA→AGA | K84E | — | ++ | SEQ ID NO: 476 |
| 85 | a | TTA→TCA | L85S | — | ++ | SEQ ID NO: 477 |

Yeast codon:

| 86 | a | GAA→GGA | E86G^{c} | E89 | ++ | SEQ ID NO: 478 |
|---|---|---|---|---|---|---|
| 88 | a | TTG→GTG | L88V | L91 | ++ | SEQ ID NO: 479 |
| 99 | b | GAA→GGA | E99G | E102 | ++ | SEQ ID NO: 480 |
| 108 | a | GCA→CCA | A108P | A111 | +++ | SEQ ID NO: 481 |
| 110 | a | GTC→GCC | V110A^{c} | V113 | +++ | SEQ ID NO: 482 |
| 112 | b | GTA→GAA | V112E | I115 | ++ | SEQ ID NO: 483 |
| 113 | b | ACG→GCG | T113A | T116 | ++ | SEQ ID NO: 484 |
| 144 | a | GGT→AGT | G144S | G147 | +++ | SEQ ID NO: 485 |
| ^{a}MMR-deficient transformants were identified by (“a”) qualitative patch assays using YBT24 or (“b”) colorimetric assay using YBT41 as described in Example 8. | | | | | | |
| ^{b}Yeast strain YBT24 containing pSH91 was transformed with pMLH1_h(41-86) containing the indicated missense mutations. Mutation frequencies were determined using a standardized MMR assay based on instability of the GT-tract in pSH91 (Example 1). To calculate the mutation defect, the mean mutation frequency confered by each variant was divided by the mutation frequency confered by the parental MLH1_h(41-86) | | | | | | |
| # gene. +, Mutation defect of 2.1 to 3.9 (18-33% loss-of-MMR function relative to the mutation frequency of the MLH1-null strain YBT24); ++, Mutation defect of | | | | | | |
| # 4.0 to 7.6 (34-66% loss-of-MMR function); +++, Mutation defect of 7.8 or greater (≧67% loss-of-MMR function). The mean mutation frequency confered by pMLH1_h(41-86) was 2.7 × 10⁻⁴ (Range: 1.1-4.4 × 10⁻⁴) The mean mutation frequency confered by the empty expression vector pMETc was 3.2 × 10⁻³ (Range: 1.9-7.0 × 10⁻³)(Mutation defect = 11.7). | | | | | | |

^{c}In addition to the indicated missense mutation the following silent alterations were observed (mutation/silent alteration): A42V/F85F; K57E/T45T; I68S/I47I and 175I; R79W/D143D; F80S/L73L; E86G/T82T and K142K; V110A/T66T.

TABLE 13
MLH1 missense mutations identified in human-yeast hybrid MLH1_h(77-134)

| | | | | | | |
|---|---|---|---|---|---|---|
| MLH1 gene or | Screening | Missense | | Corresponding human | Mutation | |
| variant codon # | method^{*} | mutation | Consequence | residue | defect^{b} | SEQ ID NO. s |

Yeast codon:

| | | | | | | |
|---|---|---|---|---|---|---|
| 30 | a | AAA→AAT | K30N | K33 | +++ | SEQ ID NO: 486 |
| 35 | a | AAT→AGT | N35S | N38 | +++ | SEQ ID NO: 487 |
| 37 | a | ATC→TTC | I37F | L40 | ++ | SEQ ID NO: 488 |
| | a | ATC→ACC | I37T | L40 | +++ | SEQ ID NO: 489 |
| 38 | a, a, b | GAT→GGT | D38G | D41 | +++ | SEQ ID NO: 490 |
| | b | GAT→GAA | D38E | D41 | +++ | SEQ ID NO: 491 |
| | b | GAT→ATT | D38N | D41 | +++ | SEQ ID NO: 492 |
| 40 | b | AAT→ATT | N40I^{c} | K43 | +++ | SEQ ID NO: 493 |
| 41 | a, a | GCT→GTT | A41V | S44 | ++ | SEQ ID NO: 494 |
| 42 | a | ACA→ATA | T42I | T45 | +++ | SEQ ID NO: 495 |
| 45 | b | GAT→GGT | D45G | Q48 | + | SEQ ID NO: 496 |
| 46 | b | ATT→AAT | I46N | V49 | +++ | SEQ ID NO: 497 |
| 49 | a | AAG→GAG | K49E | K52 | ++ | SEQ ID NO: 498 |
| 50 | a | GAA→GTA | E50V | E53 | + | SEQ ID NO: 499 |
| 52 | a, a | GGA→AGA | G52R | G55 | + | SEQ ID NO: 500 |
| 56 | b | CTT→CAT | L56H | I59 | +++ | SEQ ID NO: 501 |
| 58 | a, b | ATA→AAA | I58K | I61 | +++ | SEQ ID NO: 502 |
| 60 | a | GAT→GGT | D60G | D63 | ++ | SEQ ID NO: 503 |
| 61 | b | AAC→AGC | N61S | N64 | +++ | SEQ ID NO: 504 |
| 62 | a, b | GGA→GAA | G62E | G65 | +++ | SEQ ID NO: 505 |
| | a, a | GGA→AGA | G62R | G65 | ++ | SEQ ID NO: 506 |
| 65 | a | ATT→AAT | I65N | I68 | +++ | SEQ ID NO: 507 |
| 71 | a | CCA→CTA | P71L | D74 | ++ | SEQ ID NO: 508 |

Human codon:

| | | | | | | |
|---|---|---|---|---|---|---|
| 77 | a | TGT→CGT | C77R | — | ++ | SEQ ID NO: 509 |
| 78 | a | GAG→GTG | E78V | — | ++ | SEQ ID NO: 510 |
| 80 | a, a | TTC→CTC^{d} | F80L^{c} | — | +++ | SEQ ID NO: 511 |
| 89 | a | GAG→GTG | E89V | — | + | SEQ ID NO: 512 |
| 99 | a | TTT→ATT | F99I | — | +++ | SEQ ID NO: 513 |
| 99 | a | TTT→CTT | F99L | — | ++ | SEQ ID NO: 514 |
| 100 | b | CGA→CAA | R100Q | — | ++ | SEQ ID NO: 515 |
| 101 | a | GGT→GAT | G101D^{c} | — | +++ | SEQ ID NO: 516 |
| 103 | a | GCT→GTT | A103V | — | ++ | SEQ ID NO: 517 |
| | a, b | GCT→ACT | A103T | — | ++ | SEQ ID NO: 518 |
| | a | GCT→CCT | A103P | — | ++ | SEQ ID NO: 519 |
| 111 | a | GCT→ACT | A111T | — | +++ | SEQ ID NO: 520 |
| 114 | a | ACT→ATT | T114I | — | ++ | SEQ ID NO: 521 |
| 115 | b | ATT→AGT | I115S^{c} | — | +++ | SEQ ID NO: 522 |
| | b | ATT→AAT | I115N | — | +++ | SEQ ID NO: 523 |
| | b | ATT→TTT | 1115F | — | ++ | SEQ ID NO: 524 |
| 116 | a | ACA→TCA | T116S | — | + | SEQ ID NO: 525 |
| 118 | b | AAA→AAT | K118N | — | +++ | SEQ ID NO: 526 |
| | a | AAA→ATA | K118I | — | + | SEQ ID NO: 527 |
| 133 | a | GGA→GAA | G133E | — | ++ | SEQ ID NO: 528 |

Yeast codon:

| 136 | a | CCC→CAC | P136H | P139 | + | SEQ ID NO: 529 |
|---|---|---|---|---|---|---|
| 140 | a | GCT→GTT | A140V | A143 | ++ | SEQ ID NO: 530 |
| 144 | a | GGT→AGT | G144S | G147 | ++ | SEQ ID NO: 531 |
| ^{a}MMR-deficient transformants were identified by (“a”) qualitative patch assays using YBT24 or (“b”) colorimetric assay using YBT41 as described in Example 8. | | | | | | |
| ^{b}Yeast strain YBT24 containing pSH91 was transformed with pMLH1_h(77-134) containing the indicated missense mutations. Mutation frequencies were determined | | | | | | |
| # using a standardized MMR assay based on instability of | | | | | | |
| # the GT-tract in pSH91 (Example 1). To calculate the mutation defect, the mean mutation frequency confered by each variant was divided by the mutation frequency confered by | | | | | | |
| # the parental MLH1_h(77-134) gene. +, Mutation defect of 2.5 | | | | | | |
| # to 9.0 (9-33% loss-of-MMR function relative to the mutation frequency of the MLH1-null strain YBT24); ++, Mutation defect of 9.1 to 17.9 (34-66% loss-of-MMR | | | | | | |
| # function); +++, Mutation defect of 18.0 or greater (≧67% loss-of-MMR function). The mean mutation frequency confered by pMLH1_h(77-134) was 1.2 × 10⁻⁴ (Range: 0.6-2.4 × 10⁻⁴). The mean | | | | | | |
| # mutation frequency confered by the empty expression vector pMETc was 3.3 × 10⁻³ (Range: 1.8-7.0 × 10⁻³)(Mutation defect = 27.5) | | | | | | |

^{c}In addition to the indicated missense mutation the following silent alterations were observed (mutation/silent alteration): N40I/K134K; F80L/A92A; G101D/K54K; I115S/T116T.
^{d}The missense mutation TTC→TTA was also identified.

TABLE 14
MLH1 amino acid substitutions conferring little to no loss-of-MMR function^{a}

| | | | | | |
|---|---|---|---|---|---|
| | | | | | Is mutant residue |
| MLH1 gene and | Screening | Missense | | Corresponding | tolerated in other |
| variant codon | method^{b} | mutation | Consequence | human residue | species?^{c} |

MLH1_h(41-86):

| | | | | | | |
|---|---|---|---|---|---|---|
| 62 | (human) | b | CAA→CGA | Q62R | — | yes |
| 64 | (human) | a | AAT→GAT | N64D | — | yes |
| 71 | (human) | a | GAA→GAT | E71D | — | yes |

MLH1_h(77-134):

| 33 | (yeast) | a | ATG→TTG | M33L | I36 | yes |
|---|---|---|---|---|---|---|
| 72 | (yeast) | a | ATC→ACC | I72T | I75 | no |
| 95 | (human) | a | TCT→ACT | S95T | — | no |
| 133 | (yeast) | a | TTG→TCG | L133S | K136 | yes |
| ^{a}Mutation frequencies were measured using the standardized GT-tract instability assay as described in Example 1. Mutation frequencies were: MLH1_h(41-86) | | | | | | |
| # Q62R, 3.6 × 10⁻⁴; MLH1_h(41-86) N64D, 2.1 × 10⁻⁴; MLH1_h(41-86) E71D, 3.2 × 10⁻⁴; MLH1_h(77-134) | | | | | | |
| # M33L, 4.0 × 10⁻⁵; MLH1_h(77-134) I72T, 1.0 × 10⁻⁴; MLH1_h(77-134) S95T, 4.6 × 10⁻⁵; and MLH1_h(77-134) | | | | | | |
| # L133S, 1.5 × 10⁻⁴. These values represent mutation defects of 1.4, 0.8, 1.2, 0.3, 0.9, 0.4, and 1.3, respectively, compared to the appropriate parental hybrid gene. | | | | | | |

^{b}MMR-deficient transformants were identified by qualitative patch assays using YBT24 (“a”) or colorimetric assay using YBT41 (“b”) as described in Example 8.
^{c}As determined from the MLH1p alignment shown in FIG. 6.

1. A diagnostic method, comprising determining whether a human subject has an increased rate of accumulating genetic mutations due to the loss of DNA mismatch repair function associated with any of the following amino acid sequences:
corresponding to human MLH1: 23D (SEQ ID NO: 262), 29I (SEQ ID NO: 263), 38T (SEQ ID NO: 264), 40F (SEQ ID NO: 265), 40N (SEQ ID NO: 266), 40T (SEQ ID NO: 267), 41E (SEQ ID NO: 268), 41G (SEQ ID NO: 269), 41N (SEQ ID NO: 270), 42E (SEQ ID NO: 271), 42T (SEQ ID NO: 272), 42V (SEQ ID NO: 273), 43A (SEQ ID NO: 274), 43D (SEQ ID NO: 275), 43E (SEQ ID NO: 276), 43F (SEQ ID NO: 277), 43H (SEQ ID NO: 278), 43I (SEQ ID NO: 279), 43L (SEQ ID NO: 280), 43M (SEQ ID NO: 281), 43P (SEQ ID NO: 282), 43S (SEQ ID NO: 283), 43T (SEQ ID NO: 284), 43V (SEQ ID NO: 285), 43W (SEQ ID NO: 286), 43Y (SEQ ID NO: 287), 44D (SEQ ID NO: 288), 44G (SEQ ID NO: 289), 44K (SEQ ID NO: 290), 44M (SEQ ID NO: 291), 44N (SEQ ID NO: 292), 45I (SEQ ID NO: 293), 46T (SEQ ID NO: 294), 47S (SEQ ID NO: 295), 47T (SEQ ID NO: 296), 48G (SEQ ID NO: 297), 48Y (SEQ ID NO: 298), 49E (SEQ ID NO: 299), 49M (SEQ ID NO: 300), 49N (SEQ ID NO: 301), 51A (SEQ ID NO: 302), 51D (SEQ ID NO: 303), 55S (SEQ ID NO: 304), 56M (SEQ ID NO: 305), 56P (SEQ ID NO: 306), 57N (SEQ ID NO: 307), 59F (SEQ ID NO: 308), 59H (SEQ ID NO: 309), 59N (SEQ ID NO: 310), 59T (SEQ ID NO: 311), 61N (SEQ ID NO: 312), 63G (SEQ ID NO: 313), 63Y (SEQ ID NO: 314), 64I (SEQ ID NO: 315), 64S (SEQ ID NO: 316), 65A (SEQ ID NO: 317), 65D (SEQ ID NO: 318), 65E (SEQ ID NO: 319), 65S (SEQ ID NO: 320), 65V (SEQ ID NO: 321), 67W (SEQ ID NO: 322), 68F (SEQ ID NO: 323), 68N (SEQ ID NO: 324), 68S (SEQ ID NO: 325), 70I (SEQ ID NO: 326), 70N (SEQ ID NO: 327), 72G (SEQ ID NO: 328), 73M (SEQ ID NO: 329), 73P (SEQ ID NO: 330), 74L (SEQ ID NO: 331), 76E (SEQ ID NO: 332), 77S (SEQ ID NO: 333), 77Y (SEQ ID NO: 334), 79W (SEQ ID NO: 335), 80I (SEQ ID NO: 336), 80S (SEQ ID NO: 337), 80V (SEQ ID NO: 338), 82K (SEQ ID NO: 339), 82M (SEQ ID NO: 340), 82S (SEQ ID NO: 341), 83F (SEQ ID NO: 342), 83P (SEQ ID NO: 343), 89G (SEQ ID NO: 344), 89V (SEQ ID NO: 345), 91V (SEQ ID NO: 346), 99I (SEQ ID NO: 347), 99L (SEQ ID NO: 348), 100P (SEQ ID NO: 349), 100Q (SEQ ID NO: 350), 101D (SEQ ID NO: 351), 102D (SEQ ID NO: 352), 102G (SEQ ID NO: 353), 103T (SEQ ID NO: 354), 103V (SEQ ID NO: 355), 111P (SEQ ID NO: 356), 111T (SEQ ID NO: 357), 113A (SEQ ID NO: 358), 114I (SEQ ID NO: 359), 115E (SEQ ID NO: 360), 115F (SEQ ID NO: 361), 115N (SEQ ID NO: 362), 115S (SEQ ID NO: 363), 116A (SEQ ID NO: 364), 118N (SEQ ID NO: 365), 128P (SEQ ID NO: 366), 182G (SEQ ID NO: 367), 193P (SEQ ID NO: 368), 304V (SEQ ID NO: 601), 542P (SEQ ID NO: 369), 549P (SEQ ID NO: 370), 640S (SEQ ID NO: 602), 663G (SEQ ID NO: 371), 755S (SEQ ID NO: 372), 22A (SEQ ID NO: 598), 29S (SEQ ID NO: 373), 32V (SEQ ID NO: 374), 36L (SEQ ID NO: 375), 43C (SEQ ID NO: 376), 43G (SEQ ID NO: 377), 43N (SEQ ID NO: 378), 43Q (SEQ ID NO: 379), 43R (SEQ ID NO: 380), 62R (SEQ ID NO: 381), 64D (SEQ ID NO: 382), 71D (SEQ ID NO: 383), 75T (SEQ ID NO: 384), 95T (SEQ ID NO: 385), 136S (SEQ ID NO: 386), 141R (SEQ ID NO: 599), 160V (SEQ ID NO: 387), 272V (SEQ ID NO: 388), 286Q (SEQ ID NO: 600), 441T (SEQ ID NO: 389), 648L (SEQ ID NO: 390), and 659Q (SEQ ID NO: 391).
corresponding to human MSH2: 100/101-del (SEQ ID NO: 604), 198G (SEQ ID NO: 392), 199R (SEQ ID NO: 400), 272V (SEQ ID NO: 393), 333R (SEQ ID NO: 90), 338R (SEQ ID NO: 607), 439-del (SEQ ID NO: 609), 440P (SEQ ID NO: 610), 503P (SEQ ID NO: 394), 534C (SEQ ID NO: 611), 595R (SEQ ID NO: 614), 603N (SEQ ID NO: 615), 622T (SEQ ID NO: 616), 636P (SEQ ID NO: 99), 639R (SEQ ID NO: 93), 683R (SEQ ID NO: 395), 692R (SEQ ID NO: 95), 697R (SEQ ID NO: 96), 751R (SEQ ID NO: 97), 30L (SEQ ID NO: 603), 44M (SEQ ID NO: 396), 61P (SEQ ID NO: 397), 127S (SEQ ID NO: 398), 167H (SEQ ID NO: 399), 186S (SEQ ID NO: 89), 199W (SEQ ID NO: 605), 322V (SEQ ID NO: 606), 323C (SEQ ID NO: 401), 333Y (SEQ ID NO: 91), 349L (SEQ ID NO: 608), 390F (SEQ ID NO: 402), 390V (SEQ ID NO: 403), 562V (SEQ ID NO: 612), 583S (SEQ ID NO: 613), 609V (SEQ ID NO: 92), 647K (SEQ ID NO: 100), 656H (SEQ ID NO: 101), 683V (SEQ ID NO: 404), 688I (SEQ ID NO: 405), 691T (SEQ ID NO: 94), 722I (SEQ ID NO: 617), 729V (SEQ ID NO: 102), 735V (SEQ ID NO: 406), 770V (SEQ ID NO: 98), and 845E (SEQ ID NO: 407).

2. The diagnostic method of claim 1 which is used for determining whether a human subject has an increased susceptibility to the development of cancer associated with loss of DNA mismatch repair function, comprising determining whether the subject possesses a gene which encodes a DNA mismatch repair protein having any of the listed amino acid sequences.

3. The diagnostic method of claim 2, wherein the DNA mismatch repair protein exhibits a partial or complete loss of function and has any of the following amino acid sequences:
corresponding to human MLH1: 23D (SEQ ID NO: 262), 29I (SEQ ID NO: 263), 38T (SEQ ID NO: 264), 40F (SEQ ID NO: 265), 40N (SEQ ID NO: 266), 40T (SEQ ID NO: 267), 41E (SEQ ID NO: 268), 41G (SEQ ID NO: 269), 41N (SEQ ID NO: 270), 42E (SEQ ID NO: 271), 42T (SEQ ID NO: 272), 42V (SEQ ID NO: 273), 43A (SEQ ID NO: 274), 43D (SEQ ID NO: 275), 43E (SEQ ID NO: 276), 43F (SEQ ID NO: 277), 43H (SEQ ID NO: 278), 43I (SEQ ID NO: 279), 43L (SEQ ID NO: 280), 43M (SEQ ID NO: 281), 43P (SEQ ID NO: 282), 43S (SEQ ID NO: 283), 43T (SEQ ID NO: 284), 43V (SEQ ID NO: 285), 43W (SEQ ID NO: 286), 43Y (SEQ ID NO: 287), 44D (SEQ ID NO: 288), 44G (SEQ ID NO: 289), 44K (SEQ ID NO: 290), 44M (SEQ ID NO: 291), 44N (SEQ ID NO: 292), 45I (SEQ ID NO: 293), 46T (SEQ ID NO: 294), 47S (SEQ ID NO: 295), 47T (SEQ ID NO: 296), 48G (SEQ ID NO: 297), 48Y (SEQ ID NO: 298), 49E (SEQ ID NO: 299), 49M (SEQ ID NO: 300), 49N (SEQ ID NO: 301), 51A (SEQ ID NO: 302), 51D (SEQ ID NO: 303), 55S (SEQ ID NO: 304), 56M (SEQ ID NO: 305), 56P (SEQ ID NO: 306), 57N (SEQ ID NO: 307), 59F (SEQ ID NO: 308), 59H (SEQ ID NO: 309), 59N (SEQ ID NO: 310), 59T (SEQ ID NO: 311), 61N (SEQ ID NO: 312), 63G (SEQ ID NO: 313), 63Y (SEQ ID NO: 314), 64I (SEQ ID NO: 315), 64S (SEQ ID NO: 316), 65A (SEQ ID NO: 317), 65D (SEQ ID NO: 318), 65E (SEQ ID NO: 319), 65S (SEQ ID NO: 320), 65V (SEQ ID NO: 321), 67W (SEQ ID NO: 322), 68F (SEQ ID NO: 323), 68N (SEQ ID NO: 324), 68S (SEQ ID NO: 325), 70I (SEQ ID NO: 326), 70N (SEQ ID NO: 327), 72G (SEQ ID NO: 328), 73M (SEQ ID NO: 329), 73P (SEQ ID NO: 330), 74L (SEQ ID NO: 331), 76E (SEQ ID NO: 332), 77S (SEQ ID NO: 333), 77Y (SEQ ID NO: 334), 79W (SEQ ID NO: 335), 80I (SEQ ID NO: 336), 80S (SEQ ID NO: 337), 80V (SEQ ID NO: 338), 82K (SEQ ID NO: 339), 82M (SEQ ID NO: 340), 82S (SEQ ID NO: 341), 83F (SEQ ID NO: 342), 83P (SEQ ID NO: 343), 89G (SEQ ID NO: 344), 89V (SEQ ID NO: 345), 91V (SEQ ID NO: 346), 99I (SEQ ID NO: 347), 99L (SEQ ID NO: 348), 100P (SEQ ID NO: 349), 100Q (SEQ ID NO: 350), 101D (SEQ ID NO: 351), 102D (SEQ ID NO: 352), 102G (SEQ ID NO: 353), 103T (SEQ ID NO: 354), 103V (SEQ ID NO: 355), 111P (SEQ ID NO: 356), 111T (SEQ ID NO: 357), 113A (SEQ ID NO: 358), 114I (SEQ ID NO: 359), 115E (SEQ ID NO: 360), 115F (SEQ ID NO: 361), 115N (SEQ ID NO: 362), 115S (SEQ ID NO: 363), 116A (SEQ ID NO: 364), 118N (SEQ ID NO: 365), 128P (SEQ ID NO: 366), 182G (SEQ ID NO: 367), 193P (SEQ ID NO: 368), 304V (SEQ ID NO: 601), 542P (SEQ ID NO: 369), 549P (SEQ ID NO: 370), 640S (SEQ ID NO: 602), 663G (SEQ ID NO: 371), 755S (SEQ ID NO: 372).
corresponding to human MSH2: 100/101-del (SEQ ID NO: 604), 198G (SEQ ID NO: 392), 199R (SEQ ID NO: 400), 272V (SEQ ID NO: 393), 333R (SEQ ID NO: 90), 338R (SEQ ID NO: 607), 439-del (SEQ ID NO: 609), 440P (SEQ ID NO: 610), 503P (SEQ ID NO: 394), 534C (SEQ ID NO: 611), 595R (SEQ ID NO: 614), 603N (SEQ ID NO: 615), 622T (SEQ ID NO: 616), 636P (SEQ ID NO: 99), 639R (SEQ ID NO: 93), 683R (SEQ ID NO: 395), 692R (SEQ ID NO: 95), 697R (SEQ ID NO: 96), 751R (SEQ ID NO: 97).

4. The diagnostic method of claim 3 in which the cancer is colorectal, ovarian or endometrial in nature.

5. A method of developing data useful for determining the susceptibility of humans to the development of cancer associated with loss of DNA mismatch repair function, comprising measuring in an assay which utilizes the yeast Saccharomyces cerevisiae the loss of DNA mismatch repair function, if any, of a DNA mismatch repair protein, wherein the DNA mismatch repair protein has any of the following amino acid sequences:
corresponding to human MLH1: 23D (SEQ ID NO: 262), 29I (SEQ ID NO: 263), 38T (SEQ ID NO: 264), 40F (SEQ ID NO: 265), 40N (SEQ ID NO: 266), 40T (SEQ ID NO: 267), 41E (SEQ ID NO: 268), 41G (SEQ ID NO: 269), 41N (SEQ ID NO: 270), 42E (SEQ ID NO: 271), 42T (SEQ ID NO: 272), 42V (SEQ ID NO: 273), 43A (SEQ ID NO: 274), 43D (SEQ ID NO: 275), 43E (SEQ ID NO: 276), 43F (SEQ ID NO: 277), 43H (SEQ ID NO: 278), 43I (SEQ ID NO: 279), 43L (SEQ ID NO: 280), 43M (SEQ ID NO: 281), 43P (SEQ ID NO: 282), 43S (SEQ ID NO: 283), 43T (SEQ ID NO: 284), 43V (SEQ ID NO: 285), 43W (SEQ ID NO: 286), 43Y (SEQ ID NO: 287), 44D (SEQ ID NO: 288), 44G (SEQ ID NO: 289), 44K (SEQ ID NO: 290), 44M (SEQ ID NO: 291), 44N (SEQ ID NO: 292), 45I (SEQ ID NO: 293), 46T (SEQ ID NO: 294), 47S (SEQ ID NO: 295), 47T (SEQ ID NO: 296), 48G (SEQ ID NO: 297), 48Y (SEQ ID NO: 298), 49E (SEQ ID NO: 299), 49M (SEQ ID NO: 300), 49N (SEQ ID NO: 301), 51A (SEQ ID NO: 302), 51D (SEQ ID NO: 303), 55S (SEQ ID NO: 304), 56M (SEQ ID NO: 305), 56P (SEQ ID NO: 306), 57N (SEQ ID NO: 307), 59F (SEQ ID NO: 308), 59H (SEQ ID NO: 309), 59N (SEQ ID NO: 310), 59T (SEQ ID NO: 311), 61N (SEQ ID NO: 312), 63G (SEQ ID NO: 313), 63Y (SEQ ID NO: 314), 64I (SEQ ID NO: 315), 64S (SEQ ID NO: 316), 65A (SEQ ID NO: 317), 65D (SEQ ID NO: 318), 65E (SEQ ID NO: 319), 65S (SEQ ID NO: 320), 65V (SEQ ID NO: 321), 67W (SEQ ID NO: 322), 68F (SEQ ID NO: 323), 68N (SEQ ID NO: 324), 68S (SEQ ID NO: 325), 70I (SEQ ID NO: 326), 70N (SEQ ID NO: 327), 72G (SEQ ID NO: 328), 73M (SEQ ID NO: 329), 73P (SEQ ID NO: 330), 74L (SEQ ID NO: 331), 76E (SEQ ID NO: 332), 77S (SEQ ID NO: 333), 77Y (SEQ ID NO: 334), 79W (SEQ ID NO: 335), 80I (SEQ ID NO: 336), 80S (SEQ ID NO: 337), 80V (SEQ ID NO: 338), 82K (SEQ ID NO: 339), 82M (SEQ ID NO: 340), 82S (SEQ ID NO: 341), 83F (SEQ ID NO: 342), 83P (SEQ ID NO: 343), 89G (SEQ ID NO: 344), 89V (SEQ ID NO: 345), 91V (SEQ ID NO: 346), 99I (SEQ ID NO: 347), 99L (SEQ ID NO: 348), 100P (SEQ ID NO: 349), 100Q (SEQ ID NO: 350), 101D (SEQ ID NO: 351), 102D (SEQ ID NO: 352), 102G (SEQ ID NO: 353), 103T (SEQ ID NO: 354), 103V (SEQ ID NO: 355), 111P (SEQ ID NO: 356), 111T (SEQ ID NO: 357), 113A (SEQ ID NO: 358), 114I (SEQ ID NO: 359), 115E (SEQ ID NO:360), 115F (SEQ ID NO: 361), 115N (SEQ ID NO:362), 115S (SEQ ID NO:363), 116A (SEQ ID NO: 364), 118N (SEQ ID NO: 365), 128P (SEQ ID NO: 366), 182G (SEQ ID NO: 367), 193P (SEQ ID NO: 368), 304V (SEQ ID NO: 601), 542P (SEQ ID NO: 369), 549P (SEQ ID NO: 370), 640S (SEQ ID NO: 602), 663G (SEQ ID NO: 371), 755S (SEQ ID NO: 372), 22A (SEQ ID NO: 598), 29S (SEQ ID NO: 373), 32V (SEQ ID NO: 374), 36L (SEQ ID NO: 375), 43C (SEQ ID NO: 376), 43G (SEQ ID NO: 377), 43N (SEQ ID NO: 378), 43Q (SEQ ID NO: 379), 43R (SEQ ID NO: 380), 62R (SEQ ID NO: 381), 64D (SEQ ID NO: 382), 71D (SEQ ID NO: 383), 75T (SEQ ID NO: 384), 95T (SEQ ID NO: 385), 136S (SEQ ID NO: 386), 141R (SEQ ID NO: 599), 160V (SEQ ID NO: 387), 272V (SEQ ID NO: 388), 286Q (SEQ ID NO: 600), 441T (SEQ ID NO: 389), 648L (SEQ ID NO: 390), and 659Q (SEQ ID NO: 391).
corresponding to human MSH2: 100/101-del (SEQ ID NO: 604), 198G (SEQ ID NO: 392), 199R (SEQ ID NO: 400), 272V (SEQ ID NO: 393), 333R (SEQ ID NO: 90), 338R (SEQ ID NO: 607), 439-del (SEQ ID NO: 609), 440P (SEQ ID NO: 610), 503P (SEQ D NO: 394), 534C (SEQ ID NO: 611), 595R (SEQ ID NO: 614), 603N (SEQ ID NO: 615), 622T (SEQ ID NO: 616), 636P (SEQ ID NO: 99), 639R (SEQ ID NO: 93), 683R (SEQ ID NO: 395), 692R (SEQ ID NO: 95), 697R (SEQ ID NO: 96), 751R (SEQ ID NO: 97), 30L (SEQ ID NO: 603), 44M (SEQ ID NO: 396), 61P (SEQ ID NO: 397), 127S (SEQ ID NO: 398), 167H (SEQ ID NO: 399), 186S (SEQ ID NO: 89), 199W (SEQ ID NO: 605), 322V (SEQ ID NO: 606), 323C (SEQ ID NO: 401), 333Y (SEQ ID NO: 91), 349L (SEQ ID NO: 608), 390F (SEQ ID NO: 402), 390V (SEQ ID NO: 403), 562V (SEQ ID NO: 612), 583S (SEQ ID NO: 613), 609V (SEQ ID NO: 92), 647K (SEQ ID NO: 100), 656H (SEQ ID NO: 101), 683V (SEQ ID NO: 404), 688I (SEQ ID NO: 405), 691T (SEQ ID NO: 94), 722I (SEQ ID NO: 617), 729V (SEQ ID NO: 102), 735V (SEQ ID NO: 406), 770V (SEQ ID NO: 98), and 845E (SEQ ID NO: 407).

6. The method of claim 5 in which the cancer is colorectal, ovarian or endometrial in nature.

7. The method of claim 5 in which the yeast assay utilizes color change to measure loss of DNA mismatch repair function.

8. The method of claim 7 which utilizes the Ade2 reporter gene (SEQ ID NO: 618).

9. A yeast strain containing a DNA microsatellite sequence within the coding sequence of the native ADE2 gene, where said DNA microsatellite sequence is unstable when carried in a MMR-deficient yeast strain.

10. The yeast strain of claim 9 in which the ADE2 gene is ADE2::MS3::ADE2 (SEQ ID NO: 619).

11. The yeast strain of claim 9 which is YBT41.

12. A DNA molecule consisting of the Saccharomyces cerevisiae ADE2 gene (SEQ ID NO: 618) containing a DNA microsatellite sequence, where said DNA microsatellite sequence is unstable when carried in a MMR-deficient yeast strain.

13. The DNA molecule of claim 11 in which the ADE2 gene is ADE2::MS3::ADE2 (SEQ ID NO: 619).

14. A DNA molecule encoding any one of the following human-yeast hybrid MLH1 proteins: MLH1_(175-267) (SEQ ID NO: 40), which contains human amino acid residues 175-267 replacing yeast amino acid residues 172-267; MLH1_(175-214) (SEQ ID NO: 198), which contains human amino acid residues 175-214 replacing yeast amino acid residues 172-211; MLH11(208-267) (SEQ ID NO: 199), which contains human amino acid residues 208-267 replacing yeast amino acid residues 205-267; MLH1_(265-341) (SEQ ID NO: 200), which contains human amino acid residues 265-341 replacing yeast amino acid residues 265-341; MLH1_(265-311) (SEQ ID NO: 201), which contains human amino acid residues 265-311 replacing yeast amino acid residues 265-311; and MLH1_(298-341) (SEQ ID NO: 202), which contains human amino acid residues 298-341 replacing yeast amino acid residues 298-341.

15. A DNA molecule encoding any one of the following human-yeast hybrid MSH2 proteins: MSH2_h(621-739) (SEQ ID NO: 104), which contains human amino acid residues 621-832 replacing yeast amino acid residues 639-758; MSH2_(621-832)ins9 (SEQ ID NO: 535), which contains human amino acid residues 621-832 replacing yeast amino acid residues 639-860 and contains the peptide KNLKEQKHD (single letter amino acid code) inserted between human codons 807 and 808; and MSH2_(730-832)ins9 (SEQ ID NO: 536), which contains human amino acid residues 730-832 replacing yeast amino acid residues 749-860 and contains the peptide KNLKEQKHD (single letter amino acid code) inserted between human codons 807 and 808.

16. A variant of the hMLH1 protein which exhibits a partial or complete loss of MMR function selected from the group consisting of 29I (SEQ ID NO: 263), 38T (SEQ ID NO: 264), 40F (SEQ ID NO: 265), 40N (SEQ ID NO: 266), 40T (SEQ ID NO: 267), 41E (SEQ ID NO: 268), 41G (SEQ ID NO: 269), 41N (SEQ ID NO: 270), 42E (SEQ ID NO: 271), 42T (SEQ ID NO: 272), 42V (SEQ ID NO: 273), 43A (SEQ ID NO: 274), 43D (SEQ ID NO: 275), 43E (SEQ ID NO: 276), 43F (SEQ ID NO: 277), 43H (SEQ ID NO: 278), 43I (SEQ ID NO: 279), 43L (SEQ ID NO: 280), 43M (SEQ ID NO: 281), 43P (SEQ ID NO: 282), 43S (SEQ ID NO: 283), 43T (SEQ ID NO: 284), 43V (SEQ ID NO: 285), 43W (SEQ ID NO: 286), 43Y (SEQ ID NO: 287), 44D (SEQ ID NO: 288), 44G (SEQ ID NO: 289), 44K (SEQ ID NO: 290), 44M (SEQ ID NO: 291), 44N (SEQ ID NO: 292), 45I (SEQ ID NO: 293), 46T (SEQ ID NO: 294), 47S (SEQ ID NO: 295), 47T (SEQ ID NO: 296), 48G (SEQ ID NO: 297), 48Y (SEQ ID NO: 298), 49M (SEQ ID NO: 300), 49N (SEQ ID NO: 301), 51A (SEQ ID NO: 302), 51D (SEQ ID NO: 303), 55S (SEQ ID NO: 304), 56M (SEQ ID NO: 305), 56P (SEQ ID NO: 306), 57N (SEQ ID NO: 307), 59F (SEQ ID NO: 308), 59H (SEQ ID NO: 309), 59N (SEQ ID NO: 310), 59T (SEQ ID NO: 311), 61N (SEQ ID NO: 312), 63G (SEQ ID NO: 313), 63Y (SEQ ID NO: 314), 64I (SEQ ID NO: 315), 65A (SEQ ID NO: 317), 65D (SEQ ID NO: 318), 65E (SEQ ID NO: 319), 65S (SEQ ID NO: 320), 65V (SEQ ID NO: 321), 68F (SEQ ID NO: 323), 68S (SEQ ID NO: 325), 70I (SEQ ID NO: 326), 70N (SEQ ID NO: 327), 72G (SEQ ID NO: 328), 73M (SEQ ID NO: 329), 73P (SEQ ID NO: 330), 74L (SEQ ID NO: 331), 76E (SEQ ID NO: 332), 77S (SEQ ID NO: 333), 79W (SEQ ID NO: 335), 80I (SEQ ID NO: 336), 80S (SEQ ID NO: 337), 82K (SEQ ID NO: 339), 82M (SEQ ID NO: 340), 82S (SEQ ID NO: 341), 83F (SEQ ID NO: 342), 83P (SEQ ID NO: 343), 89G (SEQ ID NO: 344), 89V (SEQ ID NO: 345), 91V (SEQ ID NO: 346), 99I (SEQ ID NO: 347), 99L (SEQ ID NO: 348), 100Q (SEQ ID NO: 350), 101D (SEQ ID NO: 351), 102G (SEQ ID NO: 353), 103T (SEQ ID NO: 354), 103V (SEQ ID NO: 355), 111P (SEQ ID NO: 356), 111T (SEQ ID NO: 357), 113A (SEQ ID NO: 358), 114I (SEQ ID NO: 359), 115E (SEQ ID NO: 360), 115F (SEQ ID NO: 361), 115N (SEQ ID NO: 362), 15S (SEQ ID NO: 363), 116A (SEQ ID NO: 364), and 118N (SEQ ID NO: 365)

17. A variant of the hMLH1 protein which exhibits a normal level of MMR function selected from the group consisting of 36L (SEQ ID NO: 375), 43C (SEQ ID NO: 376), 43G (SEQ ID NO: 377), 43N (SEQ ID NO: 378), 43Q (SEQ ID NO: 379), 43R (SEQ ID NO: 380), 62R (SEQ ID NO: 381), 64D (SEQ ID NO: 382), 71D (SEQ ID NO: 383), 75T (SEQ ID NO: 384), 95T (SEQ ID NO: 385), and 136S (SEQ ID NO: 386)

18. A DNA molecule encoding the variant protein of claim 16 or 17.

19. A method of advising on the susceptibility of a particular human subject to the development of cancer associated with loss of DNA mismatch repair function, which utilizes any of the data developed by the method of claims 1, 3 or 5.

20. The method of claim 19 wherein the cancer is colorectal, ovarian or endometrial.
